SEQUENCE LISTING (35 sequences)

SEQ ID NO: 1 - DNA, 2709 bp, Zika virus
  source 1..2709: mol_type = genomic DNA
gggggtggaacaggagagaccctgggagagaaatggaaggcccgcctgaaccagatgtcggccctggagttctactcctacaaaaagtcaggcatcaccgaggtgtgcagagaagaagcccgccgcgccctcaaggacggtgtggcaacgggaggccatgctgtgtcccgaggaagtgcaaagctgagatggttggtggagcggggatacctgcagccccatggaaaggtcattgatcttggatgtggcagagggggctggagctactacgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgatataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctatgaggcccctacacaagggtcagcgtcctctctggtaaacggggttgtcaggctcctgtcaaaaccctgggatgtggtgactggagtcacaggaatagctatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagtaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacattcaccaacctagtggtgcagctcattcggaatatggaggctgaggaagttttggagatgcaagacttgtggctgctgcggaggccagagaaagtgaccaactggttgcagagcaacggatgggataggctcaaacgaatggcagtcagtggagatgattgcgttgtgaagccaattgatgataggttcgcacatgccctcaggttcttgaatgatatgggaaaagttaggaaggacacacaagagtggaaaccctcaacaggatgggacaactgggaagaagttccgttttgctcccaccatttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaaatacatggactacctatccacccaagtgcgctacttgggtgaagaagggtctacacctggagtgcta

SEQ ID NO: 2 - protein, 903 aa, Zika virus
GGGTGETLGEKWKARLNQMSALEFYSYKKSGITEVCREEARRALKDGVATGGHAVSRGSAKLRWLVERGYLQPHGKVIDLGCGRGGWSYYAATIRKVQEVKGYTKGGPGHEEPMLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLVNGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREYHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRPEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 3 - DNA, 2709 bp, synthetic construct
  source 1..2709: mol_type = other DNA
ggaggtggaacaggagagaccctgggagagaaatggaaggcccgcctgaatcagatgtcggccctggagttctactcctacaaaaagtcaggcatcaccgaggtgtgcagagaagaagcccgccgcgccctcaaggacggtgtggcaacgggaggccatgctgtgtcccgaggaagcgcaaagctgagatggttggtggagcggggatacctgcagccccatggaaaggtcattgaccttggatgtggcagagggggctggagctactacgccgccaccatccgcaaagttcaagaagtgaaaggatacacaaaaggaggccctggtcatgaagaacccatgttggtgcaaagctatgggtggaacatagtccgtcttaagagtggagtggacgtctttcatatggcggctgagccgtgtgacacgttgctgtgtgatataggtgagtcatcatctagtcctgaagtggaagaggcacggacgctcagagtcctctccatggtgggggattggcttgaaaaaagaccaggagccttctgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagtaacaccataaaaagtgtgtccaccacgagccagctcctcttggggcgcatggacgggcccaggaggccagtgaaatatgaggaggatgtgaacctcggctctggcacgcgggctgtggtaagctgcgctgaagctcccaacatgaaaatcattggtaaccgcattgagaggatccgcagtgagcacgcagaaacgtggttctttgacgagaaccacccatataggacatgggcttaccatggaagctacgaggcccctacacaagggtcagcgtcctctctggtgaacggggttgtcaggctcctgtcaaaaccctgggatgtggtgactggagtcacaggaatagctatgaccgacaccacaccatatggtcagcaaagagttttcaaggaaaaagtggacactagggtgccagacccccaagaaggcactcgtcaggtgatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagacagcagtggaagctgtgaacgatccaaggttctgggctctagtggacaaggaaagagagtaccacctgagaggagagtgtcagagctgtgtgtacaacatgatgggaaaaagagaaaagaaacaaggggaatttggaaaggcaaagggcagccgcgccatctggtatatgtggctaggggctagatttctagagtttgaagcccttggattcttgaacgaggatcactggatggggagagagaactcaggaggtggtgttgaagggctgggattacaaagactgggatatgtcctagaagagatgagtcgcataccaggaggaaggatgtatgcagatgacaccgctggctgggacacccgcatcagcaggtttgatctggagaatgaagctctaatcaccaaccaaatggaaaaagggcacagggccttggcattggccataatcaagtacacataccaaaataaagtggtaaaggtccttagaccagctgaaaaagggaagacagttatggacattatctcaagacaagaccaaagggggagcggacaagttgtcacttacgctcttaatacattcaccaacctagtggtgcagctcattcggaatatggaggctgaggaagttttggagatgcaagacctgtggctgctgcggaggccagagaaagtgaccaactggttgcagagcaacggatgggacaggctcaaacgaatggcagtcagtggagatgattgcgttgtgaagccaattgatgataggttcgcacatgccctcaggttcttgaatgatatgggaaaagttaggaaggacacacaggagtggaaaccctcaacaggatgggacaactgggaagaagttccgttttgctcccaccatttcaacaagctgcatctcaaggacgggaggtccattgtggttccctgccgccaccaagatgaactgattggccgggcccgcgtctcaccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaggacatgcttgtggtgtggaacagagtgtggattgaggagaacgaccacatggaagacaagaccccagttacaaaatggacagacattccctatttgggaaaaagggaagacttgtggtgtggatctctcatagggcacagaccgcgcaccacctgggctgagaacatcaaaaacacagtcaacatggtgcgcaggatcataggtgatgaagaaaaatacatggactacctatccacccaagtgcgctacttgggtgaagaagggtctacacctggagtgcta

SEQ ID NO: 4 - DNA, 2709 bp, synthetic construct
  source 1..2709: mol_type = other DNA
gggggtgggacaggagagaccctgggagagaagtggaaggcccgcctgaaccagatgtcggccctggagttctactcctacaaaaagtcaggcatcactgaggtgtgcagagaggaagcccgccgcgccctcaaggatggtgtggcaacaggaggccatgctgtgtcccgaggaagtgcaaagctcagatggttggtggagcggggatatctgcagccccatggaaaggttattgatctcggatgtggcagagggggctggagctactatgccgccaccatccgcaaagttcaggaagtgaaaggatacacaaagggaggccctggtcatgaagaacccatgttggtgcaaagctatgggtggaacatagttcgtcttaagagtggggtggacgtcttccatatggcggctgagccgtgtgacacgctgctgtgtgatataggcgagtcatcatctagtcctgaagtggaagaagctcggacgctcagagtgctctccatggtgggggactggcttgaaaaaagaccaggagccttttgcataaaagtgttgtgcccatacaccagcaccatgatggaaaccctggagcgactgcagcgtaggtatgggggaggattggtcagagtgccactctcccgcaactcaacacatgagatgtactgggtctctggagcaaaaagcaacaccataaaaagtgtgtccaccacgagccagctcctcctggggcgcatggacggccccaggaggccagtgaaatatgaggaggatgtgaatctcggctcaggcacgcgggctgtggtaagctgcgctgaggctcccaacatgaagatcatcggtaaccgcattgagaggatccgcagtgaacacgcggaaacatggttctttgatgagaaccacccatataggacatgggcctaccatggaagctatgaagcccctacacaaggatcagcgtcctccctggtaaacggggttgttaggctcctgtcaaaaccttgggatgtggtgactggagtcacaggaatagctatgaccgacaccacaccgtacggtcagcaaagagtcttcaaggaaaaagtggacactagggtgccagatccccaagaaggcactcgtcaggttatgagcatggtctcttcctggttgtggaaggagctaggcaaacataaacggccacgagtctgcaccaaagaagagttcatcaacaaggtgcgtagcaatgcagcattaggagcaatatttgaagaggaaaaagagtggaagactgctgtggaagctgtgaacgatccaaggttctgggccctagtggacaaggagagagagtaccacctgagaggagagtgccagagctgtgtgtacaacatgatgggaaaaagagaaaagaaacaaggagaatttgggaaggccaaaggcagccgcgccatctggtatatgtggttgggggctagattcctagagttcgaagcccttggattcttgaacgaggaccactggatggggagagaaaactcaggaggtggtgttgaagggttgggattacaaagactcggatacgtcctagaagagatgagccgcataccaggaggaagaatgtatgcagatgacactgctggctgggatacccgcatcagcaggtttgatctggagaatgaagctctgatcaccaaccaaatggagaaggggcacagggccctggcattggccataattaagtacacataccaaaacaaagtggtgaaggtccttagaccagctgaaaaagggaagacagtgatggacattatttcaagacaagaccaaagggggagtggacaagttgtcacttacgctctcaatacattcaccaacttggtggtgcagctcatccggaatatggaggctgaggaagtgttggagatgcaagacttgtggttgctgcggaggccagagaaagtgaccaactggctgcagagcaacggatgggatagactcaaacgaatggcagtcagtggagatgattgcgttgtgaaaccaattgatgataggttcgcacatgccctcaggttcttgaatgatatggggaaagttaggaaggacacacaagaatggaaaccctcaacaggatgggacaattgggaagaagttccgttctgctcccaccatttcaacaagctccacctcaaggacgggaggtccattgtggtcccctgccgccaccaagatgaactgattggccgggctcgcgtctctccaggggcaggatggagcatccgggagactgcttgcctggcaaaatcatatgcgcaaatgtggcagctcctttatttccacagaagggacctccgactgatggctaatgccatttgttcatctgtgccagttgactgggttccaactgggagaactacctggtcaatccatggaaagggagaatggatgactactgaagacatgctcgtggtgtggaacagagtgtggattgaggagaacgaccatatggaagacaagacccctgttacgaaatggacagacattccctatctgggaaaaagggaagacttgtggtgtggatccctcatagggcacagaccgcgcaccacttgggctgagaacattaaaaatacagtcaacatggtgcgcaggatcataggtgatgaagaaaaatacatggactatctatccacccaagtgcgctacttgggtgaagaagggtccacacctggagtgcta

SEQ ID NO: 5 - DNA, 2709 bp, synthetic construct
  source 1..2709: mol_type = other DNA
gggggtggaacaggagagaccctgggagagaaatggaaggcccgcctgaaccagatgtcggccctggagttctactcctacaaaaagtcaggcatcaccgaggtgtgcagagaagaagcccgccgcgccctcaaggacggtgtggcaacgggaggccatgctgtgtcccgaggaagtgcaaagctgagatggttggtggagcggggatacctgcagccccatggaaaggtcattgatcttggatgtggcagagggggctggagctactacgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggccgccgagccttgtgacactctactgtgtgacattggcgaatcctcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctatgaggctccaactcaaggaagcgcttcatcacttgttaatggcgtggtgcgcttgctttccaaaccatgggatgtggtgactggagtcacaggaatagctatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagtaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacattcaccaacctagtggtgcagctcattcggaatatggaggctgaggaagttttggagatgcaagacttgtggctgctgcggaggccagagaaagtgaccaactggttgcagagcaacggatgggataggctcaaacgaatggcagtcagtggagatgattgcgttgtgaagccaattgatgataggttcgcacatgccctcaggttcttgaatgatatgggaaaagttaggaaggacacacaagagtggaaaccctcaacaggatgggacaactgggaagaagttccgttttgctcccaccatttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaaatacatggactacctatccacccaagtgcgctacttgggtgaagaagggtctacacctggagtgcta

SEQ ID NO: 6 - DNA, 2709 bp, synthetic construct
  source 1..2709: mol_type = other DNA
gggggtggaacaggagagaccctgggagagaaatggaaggcccgcctgaaccagatgtcggccctggagttctactcctacaaaaagtcaggcatcaccgaggtgtgcagagaagaagcccgccgcgccctcaaggacggtgtggcaacgggaggccatgctgtgtcccgaggaagtgcaaagctgagatggttggtggagcggggatacctgcagccccatggaaaggtgattgacttgggctgtgggcggggagggtggtcctattacgccgctacaattcgcaaagttcaagaagtgaaaggatacacaaaaggaggccctggtcatgaagaacccatgttggtgcaaagctatgggtggaacatagtccgtcttaagagtggggtggacgtctttcatatggccgccgagccttgtgacactctactgtgtgacattggcgaatcctcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctatgaggctccaactcaaggaagcgcttcatcacttgttaatggcgtggtgcgcttgctttccaaaccatgggatgtggtgacaggcgttacaggaattgcaatgactgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagtaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacattcacaaacctcgtcgtccagctgataaggaacatggaagccgaagaagttctggaaatgcaagacctgtggctcctacggaggcctgaaaaggtcaccaactggttgcagagcaacggatgggataggctcaaacgaatggcagtcagtggagatgattgcgttgtcaaacccatcgacgatcggttcgcacacgccttgcgcttccttaacgatatgggcaaagtccggaaggatacacaagaatggaaacccagtacaggctgggataactgggaggaagtgcctttctgctcacatcacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaatatggttcggcgcatcataggagacgaagaaaagtacatggattacctgtccacccaagtgcgctacttgggtgaagaagggtctacacctggagtgcta

SEQ ID NO: 7 - DNA, 2709 bp, synthetic construct
  source 1..2709: mol_type = other DNA
gggggtggaacaggagagaccctgggagagaaatggaaggcccgcctgaaccagatgtcggccctggagttctactcctacaaaaagtcaggcatcaccgaggtgtgcagagaagaagcccgccgcgccctcaaggacggtgtggcaacgggaggccatgctgtgtcccgaggaagtgcaaagctgagatggttggtggagcggggatacctgcagccccatggaaaggtgatcgatctggggtgcgggagagggggatggtcatactatgcagctactatacgcaaagttcaagaagtgaaaggatacacaaaaggaggccctggtcatgaagaacccatgttggtgcaaagctatgggtggaacatagtccgtcttaagagtggggtggacgtctttcatatggccgccgagccatgcgatactctcctatgtgacataggagagagctcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctatgaggcacccactcaaggctcagcaagctctctggttaacggcgttgttagactcctaagtaagccctgggatgtggttacaggggtcactggcatagccatgaccgataccactccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagtaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacattcactaatctcgttgtgcaacttataagaaacatggaagctgaggaggttctagaaatgcaagatttgtggctgttgaggagaccagaaaaagtgaccaactggttgcagagcaacggatgggataggctcaaacgaatggcagtcagtggagatgactgcgtggttaaacctatagacgatcgctttgcacacgctctccgctttctgaacgacatgggaaaagttcggaaagatactcaagagtggaagccttctaccggatgggataactgggaagaagtgccattttgcagccaccatttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaatatggtccgccgcatcataggggacgaggagaagtacatggactacctgagtactcaggttcgctacttgggtgaagaagggtctacacctggagtgcta

SEQ ID NO: 8 - DNA, 2709 bp, synthetic construct
  source 1..2709: mol_type = other DNA
gggggtggaacaggagagaccctgggagagaaatggaaggcccgcctgaaccagatgtcggccctggagttctactcctacaaaaagtcaggcatcaccgaggtgtgcagagaagaagcccgccgcgccctcaaggacggtgtggcaacgggaggccatgctgtgtcccgaggaagtgcaaagctgagatggttggtggagcggggatacctgcagccccatggaaaggttatagatttgggatgtggaagaggaggatggagttattatgcagcaacaatacgcaaagttcaagaagtgaaaggatacacaaaaggaggccctggtcatgaagaacccatgttggtgcaaagctatgggtggaacatagtccgtcttaagagtggggtggacgtctttcatatggctgcagagccatgtgacactctcctctgtgacataggggagagttcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctatgaggcaccaactcaaggatccgcatcatctctcgttaacggcgttgtcagactactaagtaaaccatgggatgtggtgacaggggtcactggcattgccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagtaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacattcacaaacctagttgttcaactcataagaaacatggaagcagaagaagttctagaaatgcaagacctatggctactcagaagacccgaaaaagttaccaactggttgcagagcaacggatgggataggctcaaacgaatggcagtcagtggagatgattgtgttgttaaacctatagatgaccgctttgcacacgctttgagatttttgaatgacatgggaaaagttcgcaaagacacacaagaatggaaaccaagtacaggctgggataattgggaagaagtgccattttgttcacatcacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggttagacggataatcggcgacgaggagaagtacatggattatctgtctacccaagtgcgctacttgggtgaagaagggtctacacctggagtgcta

SEQ ID NO: 9 - DNA, 2709 bp, synthetic construct
  source 1..2709: mol_type = other DNA
ggaggtggaacaggagagaccctgggagagaaatggaaggcccgcctgaatcagatgtcggccctggagttctactcctacaaaaagtcaggcatcaccgaggtgtgcagagaagaagcccgccgcgccctcaaggacggtgtggcaacgggaggccatgctgtgtcccgaggaagcgcaaagctgagatggttggtggagcggggatacctgcagccccatggaaaggtgatcgacctggggtgcgggagagggggatggtcatactatgcagctactatacgcaaagttcaagaagtgaaaggatacacaaaaggaggccctggtcatgaagaacccatgttggtgcaaagctatgggtggaacatagtccgtcttaagagtggagtggacgtctttcatatggccgccgagccatgcgatactctcctatgtgacataggagagagctcatctagtcctgaagtggaagaggcacggacgctcagagtcctctccatggtgggggattggcttgaaaaaagaccaggagccttctgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagtaacaccataaaaagtgtgtccaccacgagccagctcctcttggggcgcatggacgggcccaggaggccagtgaaatatgaggaggatgtgaacctcggctctggcacgcgggctgtggtaagctgcgctgaagctcccaacatgaaaatcattggtaaccgcattgagaggatccgcagtgagcacgcagaaacgtggttctttgacgagaaccacccatataggacatgggcttaccatggaagctacgaggcacccactcaaggctcagcaagctctctggtgaacggcgttgttagactcctaagtaagccctgggatgtggttacaggggtcactggcatagccatgaccgataccactccatatggtcagcaaagagttttcaaggaaaaagtggacactagggtgccagacccccaagaaggcactcgtcaggtgatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagacagcagtggaagctgtgaacgatccaaggttctgggctctagtggacaaggaaagagagtaccacctgagaggagagtgtcagagctgtgtgtacaacatgatgggaaaaagagaaaagaaacaaggggaatttggaaaggcaaagggcagccgcgccatctggtatatgtggctaggggctagatttctagagtttgaagcccttggattcttgaacgaggatcactggatggggagagagaactcaggaggtggtgttgaagggctgggattacaaagactgggatatgtcctagaagagatgagtcgcataccaggaggaaggatgtatgcagatgacaccgctggctgggacacccgcatcagcaggtttgatctggagaatgaagctctaatcaccaaccaaatggaaaaagggcacagggccttggcattggccataatcaagtacacataccaaaataaagtggtaaaggtccttagaccagctgaaaaagggaagacagttatggacattatctcaagacaagaccaaagggggagcggacaagttgtcacttacgctcttaatacattcactaatctcgttgtgcaacttataagaaacatggaagctgaggaggttctagaaatgcaagatctgtggctgttgaggagaccagaaaaagtgaccaactggttgcagagcaacggatgggacaggctcaaacgaatggcagtcagtggagatgactgcgtggttaaacctatagacgatcgctttgcacacgctctccgctttctgaacgacatgggaaaagttcggaaagatactcaggagtggaagccttctaccggatgggataactgggaagaagtgccattttgcagccaccatttcaacaagctgcatctcaaggacgggaggtccattgtggttccctgccgccaccaagatgaactgattggccgggcccgcgtctcaccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaggacatgcttgtggtgtggaacagagtgtggattgaggagaacgaccacatggaagacaagaccccagttacaaaatggacagacattccctatttgggaaaaagggaagacttgtggtgtggatctctcatagggcacagaccgcgcaccacctgggctgagaacatcaaaaacacagtcaatatggtccgccgcatcataggggacgaggagaagtacatggactacctgagtactcaggttcgctacttgggtgaagaagggtctacacctggagtgcta

SEQ ID NO: 10 - DNA, 2709 bp, synthetic construct
  source 1..2709: mol_type = other DNA
ggaggtggaacaggagagactctgggagagaaatggaaggcccgcctgaatcagatgtcggccctggagttctactcctacaaaaagtcaggcatcaccgaggtgtgcagagaagaagcccgccgcgccctcaaggacggtgtggccacgggaggccatgctgtgtcccgaggaagcgcaaagctgagatggttggtggagagaggatacctgcagccccatggaaaggtcattgaccttggatgtggcagagggggctggagctattacgccgccaccatccgcaaagttcaagaagtgaaaggatacacaaaaggaggccccggtcatgaagaacccatgttggtgcaaagctatgggtggaacatagtccgtcttaagagtggagtggacgtctttcatatggcggctgagccgtgtgacactttgctgtgtgatataggtgagtcatcatctagtcctgaagtggaagaggcacggacactcagagtcctctccatggtgggggattggcttgaaaaaagaccaggagccttctgtataaaagtgctgtgcccatacaccagcactatgatggaaaccctggagcgactgcagcgtaggtatgggggaggactggtcagagtgccactctcccgcaactctacacatgagatgtactgggtctctggagcgaaaagtaacaccataaaaagtgtgtccaccacgagtcagctcctcttggggcgcatggacgggcccaggaggccagtgaaatatgaggaggatgtgaacctcggctctggcacacgggctgtggtaagctgcgctgaagctcccaacatgaaaatcattggtaaccgcattgagaggatccgcagtgagcacgcagaaacgtggttctttgacgagaaccacccatataggacatgggcttaccatggaagctacgaggcccctacccaagggtcagcgtcctctctggtgaacggggttgtcaggctcctgtcaaagccctgggatgtggtgactggagtcacaggaatagctatgaccgacaccacaccatatggtcaacaaagagttttcaaggaaaaagtggacactagggtgccagacccccaagaaggcactcgtcaggtgatgagcatggtctcttcctggctgtggaaagagctaggcaaacacaaacggccacgagtctgtaccaaagaagagttcatcaataaggttcgtagcaatgcagcattaggggcaatatttgaagaggaaaaagagtggaagacagcagtggaagctgtgaacgatccaaggttttgggctctagtggacaaggaaagagagtaccacctgagaggagagtgtcagagctgtgtgtacaacatgatgggaaaaagagaaaagaagcaaggggaatttggaaaggcaaagggcagccgcgccatctggtacatgtggctaggggctagatttctagagtttgaagcccttggattcttgaacgaggatcactggatgggaagagagaactcaggaggtggtgttgaagggctgggattacaaagactgggatatgtcctagaagaaatgagtcgcataccaggaggaaggatgtatgcagatgacaccgctggctgggacacccgcatcagtaggtttgatctggagaatgaagctctaatcaccaaccaaatggaaaaagggcacagggctttggcattggccataatcaagtacacataccaaaataaagtggtaaaggtcctcagaccagctgaaaaagggaagacagttatggacattatctcaagacaagaccaaagagggagcggacaagttgtcacttacgctcttaatacattcaccaacctagtggtgcagctcattcggaatatggaggctgaggaagttttggagatgcaagacctgtggctgctgaggaggccagagaaagtgaccaactggttgcagagcaacggatgggacaggctcaaacgaatggcagtcagtggagatgactgcgttgtgaagccaattgatgataggttcgcacatgccctcaggttcttgaatgatatgggaaaagttaggaaggacacacaggagtggaaaccctcaacaggatgggacaactgggaagaagttccgttttgctcccaccatttcaacaagctgcatctcaaggacgggagatccattgtggttccctgccgccaccaagatgaactgattggccgggcccgcgtctcaccaggggcgggatggagcatccgggagactgcttgtctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaggacatgcttgtggtgtggaatagagtgtggattgaggagaacgaccacatggaagacaagaccccagttacaaaatggacagacattccctatttgggaaaaagggaagacctgtggtgtggatctctcatagggcacagaccgcgcaccacctgggctgagaacatcaaaaacacagtcaacatggtgcgcaggatcataggtgatgaagaaaaatacatggactacctatccacccaagtgcgctacttgggtgaagaagggtctacacctggagtgcta

SEQ ID NO: 11 - DNA, 2709 bp, synthetic construct
  source 1..2709: mol_type = other DNA
ggtggtggaacaggagagaccctgggagagaaatggaaggcccgcctgaatcagatgtcggccctggagttctactcctacaaaaagtcaggcatcaccgaggtgtgcagagaagaagcccgccgcgccctcaaggacggtgtggcaacgggaggccatgctgtgtcccgaggatcggcaaagctgagatggttggtggagcggggatacctgcagccccatggaaaggtcattgaccttggatgtggcagagggggctggagctactacgccgccaccatccgcaaagttcaagaagtgaaaggatacacaaaaggaggccctggtcatgaagaacccatgttggtgcaaagctatgggtggaacatagtccgtcttaagagtggtgtggacgtctttcatatggcggctgagccgtgtgacacgttgctgtgtgatataggtgagtcatcatctagtcctgaagtggaagaggcacggacgctcagagtcctctccatggtgggggattggcttgaaaaaagaccaggagccttctgtataaaagtgttgtgcccatacaccagcactatgatggaaaccctggagcgactgcagcgtaggtatgggggaggactggtcagagtgccactctcccgcaactctacacatgagatgtactgggtctctggagcgaaatcgaacaccataaaaagtgtgtccaccacgagccagctcctcttggggcgcatggacgggcccaggaggccagtgaaatatgaggaggatgtgaacctcggctctggcacgcgggctgtggtaagctgcgctgaagctcccaacatgaaaatcattggtaaccgcattgagaggatccgcagtgagcacgcagaaacgtggttctttgacgagaaccacccatataggacatgggcttaccatggaagctacgaggcccctacacaagggtcagcgtcctctctggtgaacggggttgtcaggctcctgtcaaaaccctgggatgtggtgactggagtcacaggaatagctatgaccgacaccacaccatatggtcagcaaagagttttcaaggaaaaagtggacactagggtgccagacccccaagaaggcactcgtcaggtaatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagacggcagtggaagctgtgaacgatccaaggttctgggctctagtggacaaggaaagagagtaccacctgagaggagagtgtcagagctgtgtgtacaacatgatgggaaaaagagaaaagaaacaaggggaatttggaaaggcgaagggcagccgcgccatctggtatatgtggctaggggctagatttctagagtttgaagcccttggattcttgaacgaggatcactggatggggagagagaactcaggaggtggtgttgaagggctgggattacaaagactaggatatgtcctagaagagatgagtcgcataccaggaggaaggatgtatgcagatgacacggctggctgggacacccgcatcagcaggtttgatctggagaatgaagctctaatcaccaaccaaatggaaaaagggcacagggccttggcattggccataatcaagtacacataccaaaataaagtggtaaaggtccttagaccagctgaaaaagggaagacagttatggacattatctcaagacaagaccaaagggggagcggacaagttgtcacttacgctcttaatacattcaccaacctagtggtgcagctcattcggaatatggaggctgaggaagttttggagatgcaagacctatggctgctgcggaggccagagaaagtgaccaactggttgcagagcaacggatgggacaggctcaaacgaatggcagtcagtggagatgattgcgttgtgaagccaattgatgataggttcgcacatgccctcaggttcttgaatgatatgggaaaagttaggaaggacacacaggagtggaaaccctcaacaggatgggacaactgggaagaagttccgttttgctcccaccatttcaacaagctacatctcaaggacgggaggtccattgtggttccctgccgccaccaagatgaactgattggccgggcccgcgtctcgccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaggacatgcttgtggtgtggaacagagtgtggattgaggagaacgaccacatggaagacaagaccccagttacaaaatggacagacattccctatttgggaaaaagggaagacttgtggtgtggatctctcatagggcacagaccgcgcaccacctgggctgagaacatcaaaaacacagtcaacatggtgcgcaggatcataggtgatgaagaaaaatacatggactacctatccacccaagtgcgctacttgggtgaagaagggtctacacctggagtgcta

SEQ ID NO: 12 - DNA, 2709 bp, synthetic construct
  source 1..2709: mol_type = other DNA
gggggtggaacaggagagaccctgggagagaaatggaaggcccgcctgaaccagatgtcggccctggagttctactcctacaaaaagtcaggcatcaccgaggtgtgcagagaagaagcccgccgcgccctcaaggacggtgtggcaacgggaggccatgctgtgtcccgaggaagtgcaaagctgagatggttggtggagcggggatacctgcagccccatggaaaggtgatcgatctggggtgcgggagagggggatggtcatactatgcagctactatacgcaaagttcaagaagtgaaaggatacacaaaaggaggccctggtcatgaagaacccatgttggtgcaaagctatgggtggaacatagtccgtcttaagagtggggtggacgtctttcatatggccgccgagccatgcgatactctcctatgtgacataggagagagctcatctagtcctgaagtggaagaagcacggacgctcagagtcctctccatggtgggggattggcttgagaagaggccaggggcattttgcataaaagtgctgtgcccctatacctctactatgatggaaaccctggagcgactgcagcgtaggtatgggggaggactggtcagagtgccactctcccgcaactctacacatgagatgtactgggtctctggagcgaaaagcaacaccataaaatcagtttcaacaacatcacaactactactcggaagaatggacggacctcgcagaccagttaaatacgaggaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctatgaggcacccactcaaggctcagcaagctctctggttaacggcgttgttagactcctaagtaagccctgggatgtggttacaggggtcactggcatagccatgaccgataccactccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagtaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcattggatgggaagagaaaatagcggaggaggggttgaggggctcggcctccagagactgggatatgtcctcgaggagatgtcccggatccctggaggccggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacattcactaatctcgttgtgcaacttataagaaacatggaagctgaggaggttctagaaatgcaagatttgtggctgttgaggagaccagaaaaagtgaccaactggttgcagagcaacggatgggataggctcaaacgaatggcagtcagtggagatgactgcgtggttaaacctatagacgatcgctttgcacacgctctccgctttctgaacgacatgggaaaagttcggaaagatactcaagagtggaagccttctaccggatgggataactgggaagaagtgccattttgcagccaccatttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagataagactcccgtcaccaagtggaccgacatcccctatttggggaagcgggaagacttgtggtgcgggagtcttatcgggcacagaccgcgcaccacctgggctgagaacattaaaaacacagtcaatatggtccgccgcatcataggggacgaggagaagtacatggactacctgagtactcaggttcgctacttgggtgaagaagggtctacacctggagtgcta

SEQ ID NO: 13 - DNA, 2709 bp, synthetic construct
  source 1..2709: mol_type = other DNA
gggggtgggactggagagacacttggagagaaatggaaggcccggctcaaccagatgtcggccctcgagttctactcctacaaaaagtcaggcatcaccgaggtgtgcagagaagaagcccgccgcgcacttaaggacggggtggcaacgggaggccatgctgtgtccaggggaagtgcaaagctgagatggctcgtggagaggggatacctgcagccccatggaaaggtcattgatcttggatgtggcagggggggctggagctactacgccgccaccatccgcaaagttcaagaagtgaaaggatacacaaaaggaggccctggtcatgaagaacccatgttggttcaaagctatgggtggaacatagtccggctcaagagtggggtggacgtctttcatatggcggctgagccgtgtgacacacttctgtgtgatataggtgagtcatcatctagtcctgaagtggaagaagcacggactctcagggtgctctccatggttggggattggctcgaaaaaagaccaggagccttttgtataaaagtgttgtgcccatacaccagcactatgatggaaaccctcgagcggctccagcgtaggtatgggggagggctggtcagggtgccactttcccgcaactctacacatgagatgtactgggtgtctggagcgaaaagcaacaccataaaaagtgtgtccaccacgagccagctcctcttggggcgcatggacgggcccaggaggccagtgaaatatgaggaggatgtgaatctcggctctgggactcgggctgtggttagctgcgctgaagctcccaacatgaagatcattggtaaccgcattgagaggatccgcagtgagcacgcggaaacgtggttctttgacgagaaccacccatataggacttgggcttaccatggaagctatgaggcccctacacaagggtcagcgtccagcctggtaaacggggtggtcaggctcctgtcaaaaccctgggatgtggttactggggtgacaggaatagctatgaccgacaccacaccgtatggtcagcaaagggtgttcaaggaaaaagtggacactagggtgccagacccccaagaagggactcgtcaggttatgagcatggtttcttcctggctctggaaagagctaggcaaacacaaacggccacgggtgtgtaccaaagaagagttcatcaacaaggttcgtagcaatgcagcacttggggcaatctttgaagaggaaaaagagtggaagactgcagtggaagctgtgaacgatccaaggttctgggcacttgtggacaaggaaagagagtaccacctgaggggagagtgccagagctgtgtgtacaacatgatgggaaaaagagaaaagaaacaaggggaatttggaaaggccaagggcagccgcgccatctggtatatgtggctcggggctagatttctcgagttcgaagcacttggattcttgaacgaggatcactggatggggagagagaactcaggaggtggggtggaagggctcgggctacaaaggctcggatatgtcctcgaagagatgagtcgcataccaggaggaaggatgtatgcagatgacactgctggctgggacacccgcatcagcaggtttgatctcgagaatgaagcacttatcaccaaccaaatggagaaagggcacagggcacttgcacttgccataatcaagtacacataccaaaacaaagtggttaaggtccttagaccagctgaaaaagggaagacagttatggatattatttcaagacaagaccaaagggggagcggacaagtggttacttacgcacttaatacattcaccaacctggttgtgcagctcattcggaatatggaggctgaggaagttctcgagatgcaagacttgtggctcctgcggaggccagagaaagtgaccaactggctccagagcaacggatgggataggctcaaacgaatggcagtcagtggagatgattgcgtggttaagccaattgatgataggttcgcacatgcacttaggttcttgaatgatatgggaaaagttaggaaggacacacaagagtggaaaccctcaacaggatgggacaactgggaagaagttccgttttgctcccaccatttcaacaagctccatctcaaggacgggaggtccattgtggttccctgccgccaccaagatgaactgattggccgggcccgggtgtctccaggggcgggatggagcatccgggagactgcttgcctagcaaaatcatatgcgcaaatgtggcagctcctttatttccacagaagggacctccggctcatggccaatgccatttgttcatctgtgccagttgactgggtgccaactgggaggactacctggtcaatccatggaaagggagaatggatgaccactgaagatatgctggttgtgtggaacagggtgtggattgaggagaacgaccacatggaagacaagaccccagttacgaaatggactgatattccctatttgggaaaaagggaagacttgtggtgtggaagcctcatagggcacagaccgcggactacctgggctgagaacattaaaaacacagtcaacatggttcgcaggatcataggtgatgaagaaaaatacatggactacctatccacccaagtgcgctacttgggtgaagaagggtctacacctggggtgcta

SEQ ID NO: 14 - DNA, 2709 bp, synthetic construct
  source 1..2709: mol_type = other DNA
gggggtggaacaggtgaaaccttaggagaaaaatggaaggcccgcctgaaccagatgtcggccctggaattctattcctacaaaaaatcgggcattaccgaggtgtgcagagaagaagcccgacgcgcgttaaaagacggtgtagcaacgggtggccatgctgtgtcgcgaggtagtgcgaaactgcgatggttggtggagcggggttacttacagccgcatggaaaagtcattgatcttggttgtggtcgagggggctggagctactacgccgcgacgattcgcaaagttcaagaagtgaaaggttatacgaaaggtggccctggtcatgaagaacccatgttggtacaatcgtatgggtggaacatagtccgtcttaaatcgggggtagatgtatttcatatggcggctgagccgtgtgatacgttgctgtgtgatattggtgaatcgtcgtcttcgccggaagtagaagaagcacggacgttacgagtcctctccatggtaggggattggttagaaaaaagaccgggagcgttttgtattaaagtattgtgcccgtatacgtcgactatgatggaaaccctggaacgattacagcgacgatatggtggtggtctggtacgagtaccgctctcccgaaactctacgcatgaaatgtactgggtctcgggagcgaaaagcaataccattaaatcggtatcgacgacgagccagttactcttggggcgaatggacgggccccgacgaccggtaaaatatgaggaagatgtaaatttaggttctggcacgcgggcggtagtatcgtgcgctgaagcgcccaatatgaagatcattggtaaccgaattgagaggattcgaagtgagcacgcggaaacgtggttctttgacgagaatcacccgtatcgaacgtgggcttatcatggatcgtatgaggcgcctacacaagggtcggcgtcctctctggtaaacggtgtagtcaggttactgtcgaaaccctgggatgtagtaacgggtgtcacgggtattgctatgaccgataccacaccgtatggtcagcagcgagtttttaaggaaaaagtagatactcgagtaccggacccccaagaaggcactcgtcaggttatgagcatggtctcttcctggttgtggaaagaactaggcaaacacaaacggccgcgagtctgtacgaaagaagaattcatcaacaaagttcgttcgaatgcggcgttaggggcgatttttgaagaagaaaaagagtggaaaactgcagtggaagcggtaaacgatccgaggttctgggctttagtagacaaggaacgagagtaccatctgcgaggtgaatgccagagctgtgtatataatatgatgggaaaacgagaaaaaaaacagggggaatttggaaaggccaagggctcgcgcgccatctggtatatgtggctaggggctcgatttctagagttcgaagccttaggatttttgaacgaagatcactggatggggcgagagaactcgggtggtggtgttgaagggctgggtttacaaagactcggttatgtcctagaagagatgagtcgcataccaggtggtaggatgtatgcggatgacactgcgggttgggacacgcgaatcagcaggtttgatctggaaaatgaagctctaatcaccaatcaaatggagaaaggtcatagggccttggcattggccataatcaagtatacgtatcaaaacaaagtagtaaaagtccttcgaccggcggaaaaagggaagacggttatggatattatttcacgacaggatcaaaggggtagcggtcaggtagtcacgtacgcgcttaatacgttcacgaatctagtagtacagctcattcgaaatatggaggcggaagaagttttggaaatgcaagacttgtggctgctgcggaggccagaaaaagtgacgaactggttgcagagcaacggttgggataggttaaaacgaatggcagtctcgggagatgattgcgtagtgaagccgattgatgatcgatttgcgcatgcgttacgattcttaaatgatatgggaaaagtaaggaaggatacgcaagagtggaaaccgtcaacgggttgggacaactgggaagaagttccgttttgctcccaccattttaataaactccatttaaaagacggtaggtcgattgtagttccctgccgacatcaggatgaactgattggccgggcccgcgtctctccaggtgcgggttggtcgattcgagaaacggcttgcctagcgaaatcgtatgcgcaaatgtggcagctcttatattttcaccgaagggatctccgattaatggccaatgcgatttgttcatctgtaccggttgattgggttccgacgggtcgaactacgtggtcgattcatggtaaaggtgaatggatgaccactgaagatatgcttgtggtatggaaccgagtatggattgaagaaaatgaccacatggaagataaaaccccagttacgaaatggacggatattccgtatttgggaaaacgagaagatttgtggtgtggttctctcattggtcaccgaccgcgaaccacctgggcggagaatattaaaaacacggtaaacatggtgcgcaggattattggtgatgaagaaaaatacatggactacttatcgacccaagtgcgatatttgggtgaagaaggttctacgcctggtgtgcta

SEQ ID NO: 15 - DNA, 2412 bp, Zika virus
  source 1..2412: mol_type = genomic DNA
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcacagatactagtgtcggaattgttggcctcctactgaccacagccatggcagcggaagtcactagacgtgggagtgcatactatatgtacttggacagaagcgatgctggggaggccatatcttttccaaccacactggggatgaataagtgttatatacagatcatggatcttggacacatgtgtgatgccaccatgagctatgaatgccctatgctggatgagggggtagaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcagacctggttggaatcaagagaatacacaaagcacttgattagagtcgaaaattggatattcaggaaccctggcttcgcgttggcagcagctgccatcgcttggcttttgggaagctcaacgagccaaaaagtcatatacttggtcatgatactgctgattgccccggcatatagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggtcacaacaacagtcagcaacatggcggaggtaagatcctactgctatgaggcatcaatatcggacatggcttcggacagccgctgcccaacacaaggcgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccaaccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactgtgaataacaagcactggttggttcacaaggagtggttccacgacattccattaccttggcacgctggggcagacaccggaactccacactggaacaacaaagaagcactggtagagttcaaggatgcacatgccaaaaggcaaactgtcgtagttctagggagtcaagaaggagcagttcacacggcccttgctggagctctggaggctgagatggatggtgcaaagggaaggctgtcctctggccacttgaaatgtcgcctgaaaatggacaaacttagattgaagggcgtgtcatactccctgtgtgccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgcaaaccctgtaatcactgaaagcactgagaactctaagatgatgctggaacttgatccaccatttggggactcttacattgtcataggagtcggggagaagaagatcacccaccactggcacaggagtggcagcaccattggaaaagcgtttgaagccactgtgaggggtgccaagagaatggcagtcttgggagacacagcctgggactttggatcagttggaggcgctctcaactcattgggcaagggcatccatcaaatttttggagcagccttcaaatcattgtttggaggaatgtcctggttctcacaaattctcattggaacgttgctgatgtggttgggtctgaatacaaagaatggatctatttcccttatgtgcttggccttagggggagtgttgatcttcttatccacagccgtctctgctgatgtggggtgctcggtggacttctcaaaa

SEQ ID NO: 16 - protein, 804 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGTDTSVGIVGLLLTTAMAAEVTRRGSAYYMYLDRSDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTVNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCAAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSK

SEQ ID NO: 17 - DNA, 2412 bp, synthetic construct
  source 1..2412: mol_type = other DNA
atgaaaaacccaaagaagaaatccggaggattccggattgtcaatatgctaaaacgcggagtagcccgtgtaagcccctttgggggcttgaagaggctgccagccggacttctgctgggtcatggacccatcaggatggttttggcgattctagccttcttgagattcacagcaatcaagccatcactgggcctcatcaatagatggggttccgtggggaaaaaagaggctatggaaataataaagaagttcaagaaagatcttgctgccatgttgagaataatcaatgctaggaaggagaagaagagacggggcacagacactagtgtcggaatcgttggcctcctactgaccacagccatggcagcggaagtcactagacgtgggagtgcatactatatgtacttggacaggagcgatgctggggaggccatatctttcccaaccacactggggatgaacaagtgttatatacagatcatggatctcgggcacatgtgtgatgccaccatgagctatgaatgccctatgctggatgagggggtagaaccagatgacgtcgattgctggtgcaacacgacatcaacttgggttgtgtacggaacctgtcatcacaaaaaaggtgaagcacggagatctagaagagccgtgacgctcccctctcactccacaaggaagctgcaaacgcggtcgcagacttggttggaatcaagagaatacacaaagcacttgattagagttgaaaattggatattcaggaaccctggcttcgctttggcagcagctgccattgcctggcttttgggaagctcaacgagccaaaaagttatatacttggtcatgatactgctgattgccccggcatatagcatcaggtgcataggagtcagcaatagagactttgtggaaggcatgtcaggtgggacttgggttgatgttgtcttggaacatggaggctgtgtcaccgtaatggcacaggacaaaccaacagtcgacatagagctggtcacaacaacggtcagcaacatggccgaggtaagatcctactgctatgaggcatcaatatcggacatggcttcggacagccgctgcccaacacaaggcgaagcctaccttgacaagcaatcagatacccaatatgtctgcaaaagaacattagtggacagaggctggggaaatggttgtggactttttggcaaagggagcttggtgacatgcgctaagtttgcttgctccaagaaaatgaccgggaagagcatccaaccggagaatctggagtaccggataatgttgtcagtccatggctcccagcacagtgggatgattgttaatgacacaggacatgaaactgatgagaatagagcgaaagttgagataacgcccaattcaccaagagccgaagcaaccctggggggttttggaagcttgggacttgattgtgaaccaaggacaggccttgacttttcagatttgtattacttgactgtgaacaacaagcactggttggtgcacaaggagtggttccacgacatcccattaccttggcatgctggggcagacaccggaactccacactggaacaacaaagaagcattggtagagttcaaggatgcccatgccaagaggcaaactgtcgtagttctagggagccaagaaggagccgttcacacggctcttgctggagctctggaggctgagatggatggtgcaaagggaaggctgtcctctggccacttgaaatgtcgcctaaaaatggacaaacttagattgaagggcgtgtcatattccctgtgtgccgcagcgttcacattcaccaagatcccggctgaaacactgcatggaacagtcacagtggaggtacagtatgcagggacagatggaccctgcaaggttccagctcagatggcggtggacatgcagactctgaccccagttgggaggctgataaccgcaaacccggtgatcactgaaagcactgagaactcaaagatgatgctggagcttgatccaccatttggggattcttacattgtcataggagtcggggagaagaagatcacccaccactggcacaggagtggcagcaccattggaaaagcgtttgaagccactgtgaggggtgccaagagaatggcagtcttgggagacacagcctgggactttggatcagttggaggcgctctcaactcattgggcaagggcatccatcaaatttttggagcagccttcaaatcattgtttggaggaatgtcctggttctcacaaattctcattggaacgttgctgatgtggttgggtctgaatacaaagaatggatctatttcccttatgtgcttggccttagggggagtgttgatcttcttatccacagccgtctctgctgatgtggggtgctcggtggacttctcaaaa

SEQ ID NO: 18 - DNA, 2412 bp, synthetic construct
  source 1..2412: mol_type = other DNA
atgaaaaacccaaaaaagaaatccggaggattccggattgtcaatatgctaaaacgcggagtagcccgtgtgagcccctttggaggcttgaagaggttgccagccggacttctgctgggtcatgggcccatcaggatggtcttggcaattctagcctttttgagattcacggcaatcaagccatcactgggtctcatcaacagatggggttcagtggggaaaaaagaggctatggaaataataaagaagttcaagaaagatctggctgccatgctgagaataatcaatgctaggaaggagaagaagagacgaggcacagatactagtgtcggaattgttggcctcctactgactacagccatggcagcggaagtcactagacgcgggagtgcatactatatgtacttggatagaagcgatgccggggaggccatatcctttccaaccacactgggaatgaataaatgttacatacagatcatggatcttggacacatgtgtgatgccaccatgagttatgagtgccctatgctggatgagggagtagaaccagatgacgtcgattgttggtgcaacacgacgtcaacttgggttgtgtacggaacctgccatcataaaaaaggtgaagcacggagatctagaagagctgtgacgctcccctcccattccactaggaagttgcaaacgcggtcgcagacctggctagaatcaagagaatacacgaagcacctgattagagtcgaaaattggatattcaggaaccccggcttcgcgctcgcagcagctgccatcgcttggcttttgggaagctcaacgagccaaaaagtcatatacttggtcatgatactgttgattgccccggcatatagcatcaggtgcattggagtcagcaatagggactttgtggagggtatgtcaggtgggacttgggttgatgtcgtcttggaacatggaggttgtgttaccgtgatggcacaggacaaaccgactgtcgacatagagctggtcacaacgacagtcagtaacatggcggaggtaagatcctactgctacgaggcatcaatatcggacatggcttcggacagtcgctgcccaacacaaggcgaagcctaccttgacaagcaatcagacactcaatatgtctgcaaaagaacgttggtggacagaggctggggaaacggatgtggactttttggcaaagggagcctggtgacatgcgctaagtttgcatgttccaagaagatgactgggaagagcatccaaccagagaacttggagtaccggataatgctgtcagttcatggctcccagcacagcgggatgatcgttaatgacacaggacatgaaactgacgagaacagagcgaaggttgagataacgcctaattcaccaagagccgaagccaccttggggggctttggaagcctaggacttgactgtgaaccgaggacaggccttgacttttcagatctgtattacctgactgtgaataataagcactggttggttcacaaagagtggttccacgacattccattaccttggcacgctggggcagacactggaactccacactggaacaacaaagaagcactggtggagttcaaggatgcacatgccaaaaggcaaaccgtcgtagttctggggagtcaagaaggagcagttcacacggcccttgctggagctctagaggctgagatggatggagcaaagggaaggctgtcctctggccatttgaaatgtcgcctgaaaatggacaaacttagattgaagggcgtgtcatactccctgtgtgccgcggcgttcacattcaccaagatcccagctgaaacactgcacgggacagtcacagtggaggtacagtacgcagggacagatggaccttgcaaggttccagcccagatggcggtggacatgcaaactctgaccccagttgggaggttgataaccgcaaaccctgtaatcactgaaagcactgagaattctaagatgatgctggaactcgatccaccatttggggactcttacattgtcataggagttggggagaagaagatcacccatcactggcacaggagtggtagcaccattggaaaggcgtttgaggccactgtgaggggtgccaagagaatggcagtcctgggagacacagcctgggacttcggatcagttggaggcgctcttaactcactgggcaagggcattcaccaaatttttggagcagccttcaaatcattgtttggaggaatgtcctggttctcacaaattctcattggaacgttgctaatgtggttgggtctgaatacaaagaatggatctatttcccttatgtgcttggccctggggggagtgttgatcttcttatccacagccgtctctgctgatgtggggtgctcggtggacttctcaaaa

SEQ ID NO: 19 - DNA, 2412 bp, synthetic construct
  source 1..2412: mol_type = other DNA
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcacagataccagtgtgggaatcgtcgggctcctcctgacaaccgcaatggcagccgaagtcactagacgtgggagtgcatactatatgtacttggacagaagcgatgctggggaggccatatcttttccaaccacactggggatgaataagtgttatatacagatcatggatcttggacacatgtgtgatgccaccatgagctatgaatgccctatgctggatgagggggtagaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcagacctggttggaatcaagagaatacacaaagcacttgattagagtcgaaaattggatatttagaaacccagggtttgctttggccgcagccgctatcgcatggcttctaggctcatcaacgagccaaaaagtcatatacttggtcatgatactgctgattgccccggcatatagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggtcacaacaacagtcagcaacatggcggaggtaagatcctactgctatgaggcatcaatatcggacatggcttcggacagccgctgcccaacacaaggcgaagcttatcttgataagcaaagtgacacacaatatgtgtgcaaaagaacgttagtggacagaggctggggaaatggatgtggactttttggcaaagggagcctggtgacatgcgctaagtttgcatgctccaagaaaatgaccgggaagagcatccaaccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactgtgaataacaagcactggttggttcacaaggagtggttccacgacattccattaccttggcacgctggggcagacaccggaactccacactggaacaacaaagaagcactggtagagttcaaggatgcacatgccaagcggcagactgtggtcgtgctcggctctcaggaaggagcagtgcacaccgcacttgcaggagctctggaggctgagatggatggtgcaaagggaaggctgtcctctggccacttgaaatgtcgcctgaaaatggacaaacttagattgaagggcgtgtcatactccctgtgtgccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgcaaaccctgtaatcactgaaagcactgagaactctaagatgatgctggaacttgatccaccatttggggactcttacattgtcataggagtcggggagaagaagatcacccaccactggcacaggagtggcagcaccattggaaaagcgtttgaagccacagtgcggggagctaagagaatggccgtgcttggggatactgcttgggactttggatcagttggaggcgctctcaactcattgggcaagggcatccatcaaattttcggcgccgcttttaaatcacttttcggagggatgtcttggttttcacaaattctcattgggaccctcctgatgtggttggggttgaacactaaaaacgggtccatttcccttatgtgcttggccttagggggagtgttgatcttcttatccacagccgtctctgctgatgtggggtgctcggtggacttctcaaaa

SEQ ID NO: 20 - DNA, 2412 bp, synthetic construct
  source 1..2412: mol_type = other DNA
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcacagatacttccgtgggcatagtggggttgctcctgaccaccgctatggccgccgaagtcactagacgtgggagtgcatactatatgtacttggacagaagcgatgctggggaggccatatcttttccaaccacactggggatgaataagtgttatatacagatcatggatcttggacacatgtgtgatgccaccatgagctatgaatgccctatgctggatgagggggtagaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcagacctggttggaatcaagagaatacacaaagcacttgattagagtcgaaaattggatatttaggaaccccggatttgctctcgctgctgctgctattgcttggctacttggaagttcaacgagccaaaaagtcatatacttggtcatgatactgctgattgccccggcatatagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggtcacaacaacagtcagcaacatggcggaggtaagatcctactgctatgaggcatcaatatcggacatggcttcggacagccgctgcccaacacaaggcgaggcttacctcgacaaacagtctgatacccagtatgtttgtaaaagaacgttagtggacagaggctggggaaatggatgtggactttttggcaaagggagcctggtgacatgcgctaagtttgcatgctccaagaaaatgaccgggaagagcatccaaccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactgtgaataacaagcactggttggttcacaaggagtggttccacgacattccattaccttggcacgctggggcagacaccggaactccacactggaacaacaaagaagcactggtagagttcaaggatgcacatgccaaacgccagactgtggtggtgctcggctcccaagagggagccgtgcacaccgccctggccggagctctggaggctgagatggatggtgcaaagggaaggctgtcctctggccacttgaaatgtcgcctgaaaatggacaaacttagattgaagggcgtgtcatactccctgtgtgccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgcaaaccctgtaatcactgaaagcactgagaactctaagatgatgctggaacttgatccaccatttggggactcttacattgtcataggagtcggggagaagaagatcacccaccactggcacaggagtggcagcaccattggaaaagcgtttgaagccacagttaggggagcaaaaagaatggcagttttgggagacacagcttgggactttggatcagttggaggcgctctcaactcattgggcaagggcatccatcaaatttttggagcagcatttaaatcacttttcggcggcatgtcttggttctcacaaattctcattgggacccttcttatgtggctaggcctcaatactaagaacgggtccatttcccttatgtgcttggccttagggggagtgttgatcttcttatccacagccgtctctgctgatgtggggtgctcggtggacttctcaaaa

SEQ ID NO: 21 - DNA, 2412 bp, synthetic construct
  source 1..2412: mol_type = other DNA
atgaaaaacccaaaaaagaaatccggaggattccggattgtcaatatgctaaaaaggggagtagcccgtgtgagcccctttggggggctgaagaggctcccagccgggcttctgctgggtcatgggcccatcaggatggttttggcgattctagcctttttgagattcacggcaatcaagccaagcctggggctcatcaatagatggggttcagtggggaaaaaagaggctatggaaataataaagaagttcaagaaagatctggctgccatgctgagaataatcaatgctaggaaggagaagaagagaaggggcacagatactagtgtcggaattgttgggctcctactgaccacagccatggcagcggaagtcactagaagggggagtgcatactatatgtacctcgacagaagcgatgctggggaggccatatcttttccaaccacactggggatgaataagtgttatatacagatcatggatcttggacacatgtgtgatgccaccatgagctatgaatgccctatgctcgatgagggggtggaaccagatgacgtcgattgttggtgcaacacgacgtcaacttgggtggtgtacgggacttgccatcacaaaaaaggtgaagcacggagatctagaagagctgtgacactcccctcccattccactaggaagctgcaaacgcggtcgcagacctggctcgaatcaagagaatacacaaaacacttgattagggtggaaaattggatctttaggaaccctggcttcgcattggcagcagctgccatcgcttggctcttgggaagctcaacgagccaaaaagtcatatacttggttatgatactgctgattgccccggcatatagcatcaggtgcataggggtgagcaatagggactttgtggaaggtatgtcaggtgggacttgggtggatgtggttctcgaacatggaggttgtgtcaccgtaatggcacaggacaaaccgactgtcgatatagagctggttacaacaacagtcagcaacatggcggaggtaagatcctactgctatgaggcatcaatatcggatatggcttcggacagccgctgcccaacacaaggcgaagcctacctcgacaaacaatcagacactcaatatgtctgcaaaaggactttggttgacaggggctggggaaatggatgtgggctttttggcaaagggagcctggttacatgcgctaagtttgcatgctccaagaaaatgaccgggaagagcatccaaccagagaatctcgagtaccggataatgctgtcagttcatggctcccagcacagtgggatgatcgttaatgacacaggacatgaaactgatgagaatagagcgaaggttgagataacgcccaattcaccaagagccgaagccacactggggggttttggaagcctagggcttgattgtgaaccgaggactgggcttgacttttcagatttgtattacttgactgtgaataacaaacactggctcgttcacaaggagtggttccacgatattccattaccttggcacgctggggcagacaccgggactccacactggaacaacaaagaagcactggtagagttcaaggatgcacatgccaaaaggcaaactgtggttgttctagggagtcaagaaggagcagttcacacggcacttgctggagctctcgaggctgagatggatggtgcaaagggaaggctctcctctggccacttgaaatgtcgcctcaaaatggacaaacttaggctcaagggggtgtcatacagcctgtgtgccgcagcgttcacattcaccaagatcccggctgaaacactgcacgggactgtcacagtggaggtacagtacgcagggactgatggaccttgcaaggttccagctcagatggcggtggatatgcaaacactgaccccagttgggaggctcataaccgcaaaccctgtaatcactgaaagcactgagaactctaagatgatgctcgaactcgatccaccatttggggactcttacattgtcataggggtgggggagaagaagatcacccaccactggcacaggagtggcagcaccattggaaaagcgtttgaagccactgtgaggggtgccaagagaatggcagtcttgggagacacagcctgggactttggatcagttggaggcgcactcaacagcctgggcaagggcatccatcaaatctttggagcagccttcaaaagcctgtttggaggaatgtcctggttctcacaaattctcattgggactttgctgatgtggctcgggctgaatacaaagaatggatctattagccttatgtgcttggcattagggggggtgttgatctttttatccacagccgtctctgctgatgtggggtgctcggtggacttctcaaaa

SEQ ID NO: 22 - DNA, 3171 bp, Hepatitis C virus
  source 1..3171: mol_type = genomic DNA
tccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcgg

SEQ ID NO: 23 - protein, 1057 aa, Hepatitis C virus
SGSWLRDVWDWVCTILTDFKNWLTSKLFPKLPGLPFISCQKGYKGVWAGTGIMTTRCPCGANISGNVRLGSMRITGPKTCMNTWQGTFPINCYTEGQCAPKPPTNYKTAIWRVAASEYAEVTQHGSYSYVTGLTTDNLKIPCQLPSPEFFSWVDGVQIHRFAPTPKPFFRDEVSFCVGLNSYAVGSQLPCEPEPDADVLRSMLTDPPHITAETAARRLARGSPPSEASSSVSQLSAPSLRATCTTHSNTYDVDMVDANLLMEGGVAQTEPESRVPVLDFLEPMAEEESDLEPSIPSECMLPRSGFPRALPAWARPDYNPPLVESWRRPDYQPPTVAGCALPPPKKAPTPPPRRRRTVGLSESTISEALQQLAIKTFGQPPSSGDAGSSTGAGAAESGGPTSPGEPAPSETGSASSMPPLEGEPGDPDLESDQVELQPPPQGGGVAPGSGSGSWSTCSEEDDTTVCCSMSYSWTGALITPCSPEEEKLPINPLSNSLLRYHNKVYCTTSKSASQRAKKVTFDRTQVLDAHYDSVLKDIKLAASKVSARLLTLEEACQLTPPHSARSKYGFGAKEVRSLSGRAVNHIKSVWKDLLEDPQTPIPTTIMAKNEVFCVDPAKGGKKPARLIVYPDLGVRVCEKMALYDITQKLPQAVMGASYGFQYSPAQRVEYLLKAWAEKKDPMGFSYDTRCFDSTVTERDIRTEESIYQACSLPEEARTAIHSLTERLYVGGPMFNSKGQTCGYRRCRASGVLTTSMGNTITCYVKALAACKAAGIVAPTMLVCGDDLVVISESQGTEEDERNLRAFTEAMTRYSAPPGDPPRPEYDLELITSCSSNVSVALGPRGRRRYYLTRDPTTPLARAAWETVRHSPINSWLGNIIQYAPTIWVRMVLMTHFFSILMVQDTLDQNLNFEMYGSVYSVNPLDLPAIIERLHGLDAFSMHTYSHHELTRVASALRKLGAPPLRVWKSRARAVRASLISRGGKAAVCGRYLFNWAVKTKLKLTPLPEARLLDLSSWFTVGAGGGDIFHSVSRARPRSLLFGLLLLFVGVGLFLLPAR

SEQ ID NO: 24 - DNA, 3171 bp, synthetic construct
  source 1..3171: mol_type = other DNA
tctggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggcgggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcgtcggagtacgcggaggtgacgcagcatgggtcgtactcctatgtaacaggactgaccactgataatctgaaaattccttgccaactaccttctccagagtttttctcctgggtggacggtgtgcagatccataggtttgcacccacaccaaagccgtttttccgggatgaggtctcgttctgcgttggacttaattcctatgctgtcgggtcccagcttccctgtgaacctgagcccgacgcagacgtacttaggtccatgctaacagatccgccccacatcacggcggagactgcggcgcggcgcttggcacggggctcacctccatctgaggcgagctcctcagtgagccagctatcagcaccgtcgctgcgggccacgtgcaccacccacagcaacacctatgacgtggacatggtcgatgccaacctgctcatggagggcggtgtggctcagacagagcctgagtccagggtgcccgttctcgactttctcgagccaatggccgaggaagagagcgaccttgagccctcaataccatcggagtgtatgctccccaggagcgggtttccacgggccttaccggcttgggcacggcctgactacaacccgccgctcgtggaatcgtggaggaggccagattaccaaccgcccaccgttgctggttgtgctctccccccacccaagaaggccccgacgcctcccccaaggagacgccggacagtgggtctgagcgagagcaccatatcagaagccctccagcaactggccatcaagacctttggccagcccccctcgagcggagatgcaggctcgtccacgggggcgggcgccgccgaatccggcggtccgacgtcccctggggagccggccccctcagagacaggttccgcctcctctatgccccccctcgagggggagcctggagatccggacctggagtctgatcaggtagagcttcaacctcccccccagggggggggggtagctcctggttcgggctcggggtcttggtctacttgctccgaggaggacgataccaccgtgtgctgctccatgtcatactcctggaccggggctctaataactccctgtagccccgaagaggaaaaattgccaatcaaccctttgagtaactcgctgttgcgataccataacaaggtgtactgtacaacctcaaagagcgcctcacagagggctaaaaaggtaacttttgacaggacgcaagtgctcgacgctcattatgactcagtcttaaaggacatcaagctagcggcttccaaggtcagcgcaaggctcctgaccttggaggaggcgtgccagttgactccaccccattctgcaagatccaagtatggattcggtgccaaggaggtccgcagcttgtccgggagggccgttaaccacatcaagtccgtgtggaaggacctcctggaagacccacagacaccaattcccacaaccatcatggccaaaaatgaggtgttctgcgtggaccccgccaagggggggaagaaaccagctcgcctcatcgtttaccctgacctcggcgtccgggtctgcgagaaaatggctctctatgacattacacaaaagcttcctcaggcggtaatgggagcttcctatggcttccagtactcccctgcccaacgggtggagtatctcttgaaagcatgggcggaaaagaaggaccccatgggtttttcgtacgatacccgatgcttcgactcaaccgtcactgagagagacatcaggaccgaggagtccatatatcaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctctgaaagccaggggactgaggaggacgagcggaacctgagagccttcacggaggccatgaccaggtactctgccccacctggtgatccccccagaccggaatatgacctggagctaataacatcctgttcctcaaatgtgtctgtggccttgggcccgcggggccgccgcagatactacctgaccagagacccaaccactccactcgccagagctgcctgggaaacagttagacactcccctatcaattcatggctgggaaacatcatccagtatgctccaacaatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcctcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaaactcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttccttctccccgctcgg

SEQ ID NO: 25 - DNA, 3171 bp, synthetic construct
  source 1..3171: mol_type = other DNA
tccggatcctggctccgcgacgtgtgggactgggtctgcaccatcttgacagactttaaaaattggctgacctctaaactgttccccaagctgcccggccttcccttcatctcttgtcaaaaagggtacaagggtgtgtgggccggtactggcatcatgaccacgcggtgcccttgcggcgccaacatttctggcaatgtccgcctgggatctatgaggatcacagggcctaaaacctgcatgaacacctggcagggcacctttcctatcaattgctatacggagggccagtgcgcgccgaaaccccccacgaactacaagacggccatctggagggtggcggcctctgagtacgcggaggtgacgcaacatgggtcgtactcctatgtcacaggactgaccactgacaatctgaaaattccttgccaactgccttctccagagtttttcagctgggtggacggtgtgcagattcataggtttgcacccacaccgaagccgtttttccgggatgaagtctcgttctgcgttgggctgaattcctatgctgtcgggtcccagcttccctgtgaacctgagcccgacgcagacgtattgaggtccatgctaacagatccgccccatatcacggcggagactgcggcccggcgcttggcacggggatcccctccatctgaggcgagctcctcagtgagccagctatcagcaccgtcgctgcgggccacctgtaccacccacagcaacacctatgacgtggatatggtcgatgccaacctgcttatggagggcggcgtggctcagacagagcctgaatccagggtgcccgttctggattttctcgagccaatggccgaggaagagagcgaccttgagccgtcaataccatcggagtgcatgctccccaggagcgggtttccacgggccttaccggcttgggcgcggcctgactacaacccgcctctcgtggaatcgtggaggaggcccgattaccaaccgcccaccgttgccggttgtgctctcccccccccgaagaaggccccgacgcctcccccaaggagacgccggactgtgggtctgagcgagagcactatatcagaagccctccagcaactggccatcaagacctttggtcagcccccctcgagcggtgatgcaggctcgtccacgggggcgggcgccgccgaatccggcggcccgacgtcccctggtgaaccggccccctcagagacaggctccgcctcctctatgccccccctcgagggggagcctggagatcctgacctggagtctgatcaggtggagcttcaacctcccccccaaggggggggggtagctcccgggtcgggctcggggtcttggtctacatgctccgaggaggacgataccaccgtgtgttgctccatgtcatactcctggactggggctctaataactccctgtagccccgaagaggaaaagcttccaatcaaccctttgagtaattcgctgttgcgataccataataaggtgtactgtacaacatctaagagcgcctcacagagggccaaaaaggtaacttttgaccgcacgcaagtgctcgacgcccattatgactcagtcttaaaggacatcaagctagcggcttccaaggtcagcgcaaggctcctcactttggaggaggcgtgccagcttactccaccccattctgcacggtccaagtatggattcggggccaaggaggtccgcagcttgtctgggagggccgttaaccacatcaagtccgtctggaaggacctcctggaagacccacaaaccccaattcccacaaccatcatggcgaaaaatgaggtgttctgcgtggaccccgccaaggggggtaagaaaccagctcgcctcatcgtctaccctgacctcggcgtcagggtctgcgagaaaatggccctctatgacattacacaaaagctccctcaggcggtaatgggagcttcttatggcttccagtactccccagcccaacgggtggagtatcttttgaaagcatgggcggaaaagaaagaccccatgggtttttcgtatgatacccgatgcttcgactcaactgtcactgagagagacatcagaaccgaggagtccatataccaggcctgctccctgcccgaggaggcccgcactgccatacactccctgactgagagactttacgtaggagggcccatgttcaactccaagggtcaaacctgcggttacagacgttgccgcgccagcggagtgctaaccactagcatgggcaacaccatcacatgctatgtgaaagccctagcggcctgcaaggctgcggggatagttgcgcctacaatgctggtatgcggcgatgacctagtagtcatctcagagagccaggggactgaggaggacgaacggaacctgagagccttcacggaggcgatgaccaggtactctgcccctccaggtgatccccccagaccggagtatgacctggagctaataacatcctgttcttcaaatgtgtctgtggcgcttggcccgcggggccgccgcaggtactacctgaccagagacccgaccactccactcgcccgggccgcctgggaaacagttagacactccccgatcaattcatggctgggaaacataatccagtatgctccaaccatctgggttcgcatggtcctaatgacacatttcttctccattctcatggtccaagacaccctggaccagaacctcaactttgagatgtatggatcagtgtactccgtgaatcctttggatcttccagccataattgagcggttacacgggcttgacgccttctctatgcacacatactctcaccatgaactgacgcgggtggcttctgccctcagaaaacttggggcgccaccgctcagggtgtggaagagtcgggctcgcgcagtcagggcgtccctgatctcccgtggagggaaagcggccgtttgcggccgatatctctttaattgggcggtgaagaccaagcttaaactcactccattgccggaggcgcgcctactggacttatccagttggtttaccgtcggcgccggcgggggcgacatctttcacagcgtgtcgcgcgcccgaccccgctcattactcttcggcctactcctactcttcgtaggggtaggcctcttcctacttcccgctcgg

SEQ ID NO: 26 - DNA, 3171 bp, synthetic construct
  source 1..3171: mol_type = other DNA
tccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacagggttaaccactgacaatcttaagatcccatgccaactaccgtcgccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctcaccgatccaccccatataacggcggagacggcagcaaggaggttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaaattgcccatcaatccgttgagcaattcgctcctgcgctaccacaacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcgg

SEQ ID NO: 27 - DNA, 3171 bp, synthetic construct
  source 1..3171: mol_type = other DNA
tccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaattataaaaccgcgatttggcgggtagccgcttcagaatacgcggaggtgacgcagcatgggtcgtactcctatgtaacagggttaaccactgacaatcttaagatcccatgccaactaccgtcgccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctcaccgatccaccccatataacggcggagacggcagcaaggaggttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcctactgtcgctggctgcgccttgccgcctccaaagaaagcaccaacgccacccccaaggagacgccggacagtgggtctgagcgagagcaccatatcagaggccttgcagcaacttgctattaagaccttcggtcaaccgccgtcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaaattgcccatcaatccgttgagcaattcgctcctgcgctaccacaacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaaaaagccagctcgccttatcgtgtatcccgacttgggtgttcgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgaccgagagactttatgtggggggtcccatgtttaatagcaaggggcaaacctgcggttacagacgttgccgcgccagcggggtgctgaccaccagcatgggtaataccatcacatgctacgtgaaggccttagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtctccgtcgccctgggcccacggggcagaaggagatattacctcaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggggggaaggccgctgtttgtggccggtacctctttaactgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcgg

SEQ ID NO: 28 - DNA, 3171 bp, synthetic construct
  source 1..3171: mol_type = other DNA
tccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaattataaaaccgcgatttggagggtagctgcttcagaatacgcggaagtgacgcagcatgggtcgtactcctatgtaacagggttgaccactgacaatctgaagatcccgtgccagctaccgtcgccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctcaccgatcccccccacataacggcggaaacggctgcccgtaggttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgccaactgtggctggctgcgccttgccgcctccaaaaaaagcgccgacgccacccccaaggagacgccggacagtgggtctgagcgagagcaccatatcagaggccctgcaacagctcgctatcaagaccttcggccaaccgccttcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaaattgccaattaaccccctgagcaactcgctcctgcgttatcacaacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaagccagctcgccttatcgtatatccagatctgggagtgcgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagcggctttacgttgggggccctatgtttaactcaaaggggcaaacctgcggttacagacgttgccgcgccagcggggtgctgaccactagcatgggcaacaccatcacttgctacgtaaaggccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtccgtcgccctgggccctcggggaagaagacggtactacctcaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaggctgccgtatgtggcagatacctctttaactgggcagtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcgg

SEQ ID NO: 29 - DNA, 3171 bp, synthetic construct
  source 1..3171: mol_type = other DNA
tccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaattataaaaccgcgatttggagggtagccgcatcagaatacgcggaagtgacgcagcatgggtcgtactcctatgtaacaggcctgaccactgacaatcttaagattccgtgccagctgccatcgccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctcacagatccaccccacattacagcagagacggcagctcgtaggttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcctactgtggcggggtgtgccttgccacctccaaaaaaggcgccaacgccccccccaaggagacgccggacagtgggtctgagcgagagcaccatatcagaagccttgcaacaactcgccattaaaaccttcggtcaaccaccatcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaaactgccaatcaaccctctgagcaactctctcctacgttatcacaacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaagccagctcgccttatcgtatatcccgatttgggagttcgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgaccgagcggctctacgttgggggccccatgttcaatagcaaggggcaaacctgcggttacagacgttgccgcgccagcggggtactgaccaccagcatgggtaacaccatcacttgctacgtaaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtccgtcgcgctgggtcctagaggaagaagaagatactacctcaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggaggcaaagctgctgtctgtggccggtacctctttaattgggcagtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcgg

SEQ ID NO: 30 - DNA, 3171 bp, synthetic construct
  source 1..3171: mol_type = other DNA
tccggatcctggctccgcgacgtgtgggactgggtctgcacgatcttgacagacttcaaaaactggctgacctccaaattgttccccaagctgcccggcctccccttcatctcttgtcaaaaggggtacaagggtgtctgggccggcactggcatcatgaccacgcgctgcccttgtggggccaacatctctggcaatgtccgcctgggctctatgaggatcacagggcctaaaacctgcatgaacacctggcaggggacctttcctatcaactgctacacggaaggccagtgcgcgccgaaaccccccacgaactacaagaccgcgatctggagggtggcggcctcggagtacgcggaggtgacgcagcatgggtcttactcctatgtaacaggactgaccactgacaatctgaaaattccttgccaactaccttctccagagtttttctcctgggtggacggtgtgcagatccataggtttgcaccgacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccctccgaggcgagctcctcagtgagccagctatcagcaccgtcgctgcgggcgacatgcaccacccacagcaacacctatgacgtggacatggtcgatgccaacctgctcatggagggcggtgtggctcagacagagcctgagtccagggtgcccgttctggactttctcgagccgatggccgaggaagagagcgaccttgagccctccataccatcggagtgcatgctccccaggagtgggtttccacgggccttaccggcttgggcacggcctgactacaaccccccactcgtggaatcgtggaggaggccagattaccaaccgcccaccgttgctggttgtgctctcccccccccaaagaaggccccaacgcccccaccaaggagacgccggacagtgggtctgagcgagagcaccatatcagaagccctccagcaactggccatcaagacctttggccagcccccatcgagcggtgatgcaggctcgtccacgggggcgggcgccgccgaatccggcggtccgacctcccctggtgagccggccccatcagagacaggttccgcctcctccatgccccccctcgagggggagcctggagatccggacctggagtctgatcaggtagagcttcaacctcccccccaaggggggggggtagctcccggttcgggctcggggtcttggtctacttgcagcgaggaggatgacaccaccgtctgctgctcgatgtcttactcctggaccggggctctaataaccccatgcagccccgaagaggaaaagttgccaatcaaccctttgagtaactcgctgttgcgataccataacaaggtgtactgtacaacctccaagagcgcctcacagagggctaaaaaggtaacttttgacaggacgcaagtgctcgacgcccattacgactcagtcttaaaggacatcaagctagcggcttccaaggtcagcgcaaggctcctcaccttggaggaggcgtgccagttgactcccccacattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaacgaggtgttctgcgtggaccccgccaaagggggtaagaaaccagctcgcctcatcgtttaccctgacctcggcgtccgggtctgcgagaaaatggccctctacgacattacacaaaagcttcctcaggcggtaatgggagcctcctatggcttccagtactccccagcccaacgggtggagtatctcctcaaagcatgggcggaaaagaaggaccccatgggtttttcgtatgacacccgatgcttcgactcaaccgtcactgagagagacatcaggaccgaggagtccatataccaggcctgctccctgcccgaggaggcccgcactgccatacactcgctgactgagcggctttacgtaggagggcccatgttcaacagcaaagggcaaacctgcggctacagacgttgccgcgccagcggggtgctaaccactagcatgggtaacaccatcacatgctatgtgaaagccctagcggcctgcaaggctgcggggatagttgcgccgacaatgctggtatgcggcgacgacctagtagtcatctcagaaagccaagggactgaggaggatgagcggaacctgagagccttcacggaggccatgaccaggtactctgcccctcctggtgatcccccaagaccggaatacgacctggagctaataacatcctgttcctccaatgtgtctgtggcgttgggcccgcggggccgccgcagatactacctgaccagagacccaaccaccccactcgcccgggctgcctgggaaacagttagacactccccaatcaattcatggctgggaaacatcatccagtatgctccaaccatctgggttcgcatggttctaatgacacacttcttctccatcctcatggtccaggataccctggaccagaacctcaacttcgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtctggaagagtcgggctcgcgcagtcagggcctccctcatctcccgtggagggaaagcggccgtctgcggccgatatctcttcaactgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctcctccccgctcgg

SEQ ID NO: 31 - DNA, 3171 bp, synthetic construct
  source 1..3171: mol_type = other DNA
tccggatcctggctccgcgacgtgtgggattgggtttgtaccatcttgacagattttaaaaattggctgacctctaaattgttccccaagctgcccggcctcccatttatctcttgtcaaaagggttacaagggtgtgtgggccggcactggtatcatgaccacgcgctgtccttgtggtgccaacatctctggtaatgtacgcctgggctctatgaggattacagggcctaaaacctgtatgaatacctggcaggggacctttccaattaattgctacactgagggtcagtgcgcgccaaaaccccccacgaattataagaccgcaatctggagggtagcggcctcggagtatgcagaagtgacgcagcatgggtcgtactcctatgtaacaggactgaccactgataatctgaaaattccttgtcaactaccttctccagaatttttttcctgggtggatggtgtacagatccataggtttgcacccacaccaaagccgtttttccgagatgaagtctcgttttgcgttgggttaaattcctatgctgtcggttcccaacttccctgtgaaccagaaccagatgcagacgtattgaggagtatgctaacagatccaccccatatcacggcagaaactgcggcgcggcgcttggcacggggttcaccaccatctgaggcaagttcctcagtgagtcaactatcagcaccgtcgctgcgagccacctgtaccacccacagcaacacctatgatgtggacatggtcgatgcaaatttactcatggaaggcggtgtggcacagacagaacctgaatcccgagtgcccgttctggattttttagagccaatggccgaggaagagagcgaccttgaacccagtataccatcggaatgcatgctccccaggagcgggtttccacgggccttaccagcttgggcacggccagattataatccgccgctcgtggaatcgtggaggaggccagattatcaaccgcccaccgttgctggttgtgctctccccccacccaagaaggccccgacgcctcccccacgaagacgccggacagtgggtctgagcgagagcaccatatcagaagccctccagcaactggccatcaaaacctttggccagcccccctcgagcggtgatgcaggctcgtccacgggggcgggcgccgccgaatccggcggtccgacgtcccctggtgagccggcccccagtgaaacaggttccgcctccagtatgccaccactcgaaggtgaacctggtgatccggatctggagtctgatcaggtagagcttcaacctcccccccagggtgggggggtagctcccggttcgggctcggggtcttggtctacttgctccgaggaggacgataccactgtgtgttgttccatgtcatattcctggactggtgctctaataactccctgtagccccgaagaagaaaaattgccaattaaccctttgagtaattcgctgttgcgataccataataaggtgtattgtacaacatcaaagagcgcctcacagagggctaaaaaagtaacttttgataggacgcaagtgctcgatgcccattatgactcagtcttaaaagatatcaaactagcggcatccaaggtcagcgcaaggctcttaaccttggaagaagcgtgtcagttaactccaccccattctgcaagatccaagtatggttttggtgcaaaagaagtacgcagcttgtccgggcgagcagttaaccatattaagagtgtatggaaggatctcttagaagatccacaaactccaattcccacaactattatggccaaaaatgaagtgttttgcgtggaccccgccaaagggggtaagaaaccagcacgactcattgtttatcctgatctcggcgtccgggtatgtgaaaaaatggcactctatgatattacacaaaagcttcctcaggcagtaatgggagcttcctatggctttcaatattcccctgcccaacgagtggagtatctcttgaaagcatgggcggaaaaaaaggaccccatgggtttttcgtatgatactcgatgttttgattcaaccgtaactgaaagagatattaggaccgaagagtccatatatcaagcctgttccctgcccgaggaggcacgcactgccatacactcgttaactgaaagattatatgtaggtgggccaatgttcaatagcaagggtcaaacctgtggttacagacgttgtcgagccagtggggtgctaaccactagtatgggtaatactatcacatgttatgtgaaagcactagcggcatgcaaggctgcggggattgttgcacccacaatgctggtatgcggcgatgatctagtagtaatttcagaaagtcaggggactgaggaagatgagcggaacctgcgagcctttactgaagcaatgactcgatattctgcaccaccaggtgatccccccagaccagaatatgatctggaactaattactagttgtagtagtaatgtatctgtagcgttgggtccgcggggccgccgcagatattatctgactagagatccaaccactccactcgcccgggcagcatgggaaactgttagacatagtccaatcaatagttggctgggtaatatcattcagtatgctccaaccatatgggttcgcatggtcctaatgacacattttttttccattctcatggtccaagacaccttagaccagaacctcaactttgagatgtatggttcagtatatagtgtgaatccattggatttaccagccattattgagaggttacatgggcttgacgcatttagtatgcatacatatagtcatcacgaattaactcgagtagcttcagccttacgaaaattaggtgcgccaccattacgagtgtggaaaagtcgagcacgcgcagtaagggcatccctcatcagtcgtggtggtaaagcggccgtttgtggtcgatatttatttaattgggcagtaaagactaaactcaaattaactccattgccggaggcgcgcctactggatttaagtagttggtttaccgtcggtgccggtggtggtgatatttttcatagtgtgagtcgcgcacgaccacgatcattactcttcggtttattattattattcgtaggtgtaggtttattcctattaccagctcga

SEQ ID NO: 32 - DNA, 10800 bp, Zika virus
  source 1..10800: mol_type = genomic DNA
gatctgtgtgaatcagactgcgacagttcgagtttgaagcgagagctaacaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcacagatactagtgtcggaattgttggcctcctactgaccacagccatggcagcggaagtcactagacgtgggagtgcatactatatgtacttggacagaagcgatgctggggaggccatatcttttccaaccacactggggatgaataagtgttatatacagatcatggatcttggacacatgtgtgatgccaccatgagctatgaatgccctatgctggatgagggggtrgaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcagacctggttggaatcaagagaatacacaaagcacttgattagagtcgaaaattggatattcaggaaccctggcttcgcgttggcagcagctgccatcgcttggcttttgggaagctcaacgagccaaaaagtcatatacttggtcatgatactgctgattgccccggcatatagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggtcacaacaacagtcagcaacatggcggaggtaagatcctactgctatgaggcatcaatatcggacatggcttcggacagccgctgcccaacacaaggcgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccaaccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggatgcacatgccaaaaggcaaactgtcgtagttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccctgtgtgccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgcaaaccctgtaatcactgaaagcactgagaactctaagatgatgctggaacttgatccaccatttggggactcttacattgtcataggagtcggggagaagaagatcacccaccactggcacaggagtggcagcaccattggaaaagcgtttgaagccactgtgaggggtgccaagagaatggcagtcttgggagacacagcctgggactttggatcagttggaggygctctcaactcattgggcaagggcatccatcaaatttttggagcagccttcaaatcattgtttggaggaatgtcctggttctcacaaattctcattggaacgttgctgatgtggttgggtctgaatacaaagaatggatctatttcccttatgtgcttggccttagggggagtgttgatcttcttatccacagccgtctctgctgatgtggggtgctcggtggacttctcaaaaaaggaaacgagatgcggtacaggggtgttcgtctacaacgacgttgaagcctggagagacaggtacaagtaccatcctgactcccctcgtagattggcagcagcagtcaagcaagcctgggaagatgggatctgtgggatctcctctgtttcaagaatggagaacatcatgtggagatcagtagaaggggagctcaacgcaatcctagaagagaatggagttcaactgacggtcgtagtgggatctgtaaaaaaccccatgtggagaggtccacagagattgcccgtgcctgtgaacgaactgccccacggctggaaggcttggggaaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattactcattagagtgtgatccagccgtcataggaacagctgttaagggaaaggaggctgtacacagtgatctaggctactggattgagagtgagaagaacgacacatggaggctgaagagggcccacctgatcgagatgaaaacatgtgaatggccaaagtcccacacattgtggacagatggaatagaagaaagtgatctgatcatacccaagtctttagctgggccactcagccatcacaacaccagagagggttacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagtaacttagtaaggtcaatggtgactgcaggatcaactgatcacatggatcacttctcccttggagtgcttgtgattctgctcatggtgcaggaagggctgaagaagagaatgaccacaaagattatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttctgcaaactgcgatctccgccttggaaggcgacctgatggttctcatcaatggttttgctttggcctggttggcaatacgagcgatggttgttccacgcactgacaatatcaccttggcaatcctggctgctctgacaccactggcccggggcacactgcttgtggcgtggagagcaggccttgctacttgcggggggttcatgctcctctctctgaaggggaaaggcagtgtgaagaagaacttaccatttgtcatggccctgggactaaccgctgtgaggctggtcgaccccatcaacgtggtgggactgctgttgctcacaaggagtgggaagcggagctggccccctagtgaagtactcacagctgttggcctgatatgcgcattggctggagggtttgccaaggcggatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcactagatgagagtggtgatttctccctggtggaggatgatggtccccccatgagagagatcatactcaaagtggtcctgatgaccatctgtggcatgaacccaatagccataccctttgcagctggagcgtggtacgtatatgtgaagactggaaaaaggagtggtgctctatgggatgtgcctgctcccaaggaagtgaaaaagggggagaccacagatggagtgtacagagtaatgactcgtagactgctaggttcaacacaagttggagtgggagtcatgcaagagggggtctttcacactatgtggcacgtcacaaaaggatccgcgctgaggagcggtgaagggagacttgatccatactggggagatgtcaagcaggatctggtgtcatattgtggtccatggaagctagatgccgcctgggacgggcacagcgaggtgcagctcttggccgtgccccccggagagagagcgaggaacatccagactctgcctggaatatttaagacaaaggatggggacattggagcggttgcgctggactacccagcaggaacttcaggatctccaatcctagacaagtgtgggagagtgataggactctatggcaatggggtcgtgatcaaaaatgggagttatgttagtgccatcacccaagggaggagggaggaagagactcctgttgagtgcttcgagccttcgatgctgaagaagaagcaactaactgtcttagacttgcaccctggagctgggaaaaccaggagagttcttcctgaaatagtccgtgaagccataaaaacaagactccgcactgtgatcttggctccaaccagggttgtcgctgctgaaatggaggaagcccttagggggcttccagtgcgttatatgacaacagcagtcaatgtcacccattctgggacagaaatcgttgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggccgccatcttcatgaccgccacgccaccaggaacccgtgacgcattcccggactccaactcaccaatcatggacaccgaagtggaagtcccagagagagcctggagctcaggctttgattgggtgacggatcattctggaaaaacagtttggtttgttccaagcgtgaggaacggcaatgagatcgcagcttgtctgacgaaggctggaaaacgggtcatacagctcagcagaaagacttttgagacagagttccagaaaacaaaacatcaagagtgggacttcgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgatgaagaccatgcacactggcttgaagcaagaatgctccttgacaacatttacctccaagatggcctcatagcctcgctctatcgacctgaggccgacaaggtagcagctattgagggagagttcaagcttaggacggagcaaaggaagacctttgtggaactcatgaaaagaggagatcttcctgtttggctggcctatcaggttgcatctgccggaattacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcagcatttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgcggaacaagggcatagggaagatgggctttggaatggtgactcttggggccagcgcatggctcatgtggctctcggaaattgagccagccagaattgcatgtgtcctcattgttgtgttcctactgctggtggtgctcatacctgagccagaaaagcaaagatctccccaggacaaccaaatggcaatcatcatcatggtagcagtgggtcttctgggcttgattactgccaatgaactcggatggttggagagaacaaagagtgacctaagccatctaatgggaaggagagaggagggggcaaccataggattctcaatggacattgacctgcggccagcctcagcttgggctatctatgctgccctgacaactttcattaccccagccgtccagcatgcagtgaccacttcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagctgcaacttccactttgtgggaaggctctccgaacaagtactggaactcctctacagctacttcactgtgtaacatttttaggggaagttacttggctggagcttctctaatctacacagtaacaagaaacgctggcttggtcaagagacgtgggggtggaacaggagagaccctgggagagaaatggaaggcccgcctgaaccagatgtcggccctggagttctactcctacaaaaagtcaggcatcaccgaggtgtgcagagaagaagcccgccgcgccctcaaggacggtgtggcaacgggaggccatgctgtgtcccgaggaagtgcaaagctgagatggttggtggagcggggatacctgcagccccatggaaaggtcattgatcttggatgtggcagagggggctggagctactacgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgatataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctatgaggcccctacacaagggtcagcgtcctctctggtaaacggggttgtcaggctcctgtcaaaaccctgggatgtggtgactggagtcacaggaatagctatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagtaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacattcaccaacctagtggtgcagctcattcggaatatggaggctgaggaagttttggagatgcaagacttgtggctgctgcggaggccagagaaagtgaccaactggttgcagagcaacggatgggataggctcaaacgaatggcagtcagtggagatgattgcgttgtgaagccaattgatgataggttcgcacatgccctcaggttcttgaatgatatgggaaaagttaggaaggacacacaagagtggaaaccctcaacaggatgggacaactgggaagaagttccgttttgctcccaccatttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaaatacatggactacctatccacccaagtgcgctacttgggtgaagaagggtctacacctggagtgctataagcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgadtagcggcggccggtgtggggaaatccatgggtct

SEQ ID NO: 33 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGTDTSVGIVGLLLTTAMAAEVTRRGSAYYMYLDRSDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCAAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPHGKVIDLGCGRGGWSYYAATIRKVQEVKGYTKGGPGHEEPMLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLVNGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREYHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRPEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 34 - DNA, 9666 bp, Hepatitis C virus
  source 1..9666: mol_type = genomic DNA
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccctctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 35 - protein, 3029 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYALYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGAIVVSMTGRDRTEQAGEVQILSTVSQSFLGTTISGVLWTVYHGAGNKTLAGLRGPVTQMYSSAEGDLVGWPSPPGTKSLEPCKCGAVDLYLVTRNADVIPARRRGDKRGALLSPRPISTLKGSSGGPVLCPRGHVVGLFRAAVCSRGVAKSIDFIPVETLDVVTRSPTFSDNSTPPAVPQTYQVGYLHAPTGSGKSTKVPVAYAAQGYKVLVLNPSVAATLGFGAYLSKAHGINPNIRTGVRTVMTGEAITYSTYGKFLADGGCASGAYDIIICDECHAVDATSILGIGTVLDQAETAGVRLTVLATATPPGSVTTPHPDIEEVGLGREGEIPFYGRAIPLSCIKGGRHLIFCHSKKKCDELAAALRGMGLNAVAYYRGLDVSIIPAQGDVVVVATDALMTGYTGDFDSVIDCNVAVTQAVDFSLDPTFTITTQTVPQDAVSRSQRRGRTGRGRQGTYRYVSTGERASGMFDSVVLCECYDAGAAWYDLTPAETTVRLRAYFNTPGLPVCQDHLEFWEAVFTGLTHIDAHFLSQTKQAGENFAYLVAYQATVCARAKAPPPSWDAMWKCLARLKPTLAGPTPLLYRLGPITNEVTLTHPGTKYIATCMQADLEVMTSTWVLAGGVLAAVAAYCLATGCVSIIGRLHVNQRVVVAPDKEVLYEAFDEMEECASRAALIEEGQRIAEMLKSKIQGLLQQASKQAQDIQPAMQASWPKVEQFWARHMWNFISGIQYLAGLSTLPGNPAVASMMAFSAALTSPLSTSTTILLNIMGGWLASQIAPPAGATGFVVSGLVGAAVGSIGLGKVLVDILAGYGAGISGALVAFKIMSGEKPSMEDVINLLPGILSPGALVVGVICAAILRRHVGPGEGAVQWMNRLIAFASRGNHVAPTHYVTESDASQRVTQLLGSLTITSLLRRLHNWITEDCPIPCSGSWLRDVWDWVCTILTDFKNWLTSKLFPKLPGLPFISCQKGYKGVWAGTGIMTTRCPCGANISGNVRLGSMRITGPKTCMNTWQGTFPINCYTEGQCAPKPPTNYKTAIWRVAASEYAEVTQHGSYSYVTGLTTDNLKIPCQLPSPEFFSWVDGVQIHRFAPTPKPFFRDEVSFCVGLNSYAVGSQLPCEPEPDADVLRSMLTDPPHITAETAARRLARGSPPSEASSSVSQLSAPSLRATCTTHSNTYDVDMVDANLLMEGGVAQTEPESRVPVLDFLEPMAEEESDLEPSIPSECMLPRSGFPRALPAWARPDYNPPLVESWRRPDYQPPTVAGCALPPPKKAPTPPPRRRRTVGLSESTISEALQQLAIKTFGQPPSSGDAGSSTGAGAAESGGPTSPGEPAPSETGSASSMPPLEGEPGDPDLESDQVELQPPPQGGGVAPGSGSGSWSTCSEEDDTTVCCSMSYSWTGALITPCSPEEEKLPINPLSNSLLRYHNKVYCTTSKSASQRAKKVTFDRTQVLDAHYDSVLKDIKLAASKVSARLLTLEEACQLTPPHSARSKYGFGAKEVRSLSGRAVNHIKSVWKDLLEDPQTPIPTTIMAKNEVFCVDPAKGGKKPARLIVYPDLGVRVCEKMALYDITQKLPQAVMGASYGFQYSPAQRVEYLLKAWAEKKDPMGFSYDTRCFDSTVTERDIRTEESIYQACSLPEEARTAIHSLTERLYVGGPMFNSKGQTCGYRRCRASGVLTTSMGNTITCYVKALAACKAAGIVAPTMLVCGDDLVVISESQGTEEDERNLRAFTEAMTRYSAPPGDPPRPEYDLELITSCSSNVSVALGPRGRRRYYLTRDPTTPLARAAWETVRHSPINSWLGNIIQYAPTIWVRMVLMTHFFSILMVQDTLDQNLNFEMYGSVYSVNPLDLPAIIERLHGLDAFSMHTYSHHELTRVASALRKLGAPPLRVWKSRARAVRASLISRGGKAAVCGRYLFNWAVKTKLKLTPLPEARLLDLSSWFTVGAGGGDIFHSVSRARPRSLLFGLLLLFVGVGLFLLPAR

(19) United States
(12) Patent Application Publication
(10) Pub. No.: US 2026/0185060 A1
(43) Pub. Date: Jul. 2, 2026

(54) ZIKA AND HCV ATTENUATED VIRUSES AND USE THEREOF

(71) Applicant: BAR-ILAN UNIVERSITY, Ramat Gan (IL)

(72) Inventors: Tamir TULLER, Tel Aviv (IL); Meital GAL-TANAMY, Kfar Vradim (IL)

(21) Appl. No.: 19/541,345

(22) Filed: Feb. 16, 2026

Related U.S. Application Data
(63) Continuation-in-part of application No. PCT/IL2023/050857, filed on Aug. 15, 2023.
(60) Provisional application No. 63/878,149, filed on Sep. 9, 2025.

(51) Int. Cl. C12N 7/04; A61K 39/00; A61K 39/29; A61P 31/14; C07K 14/18; C12Q 1/68; G16B 15/10; G16B 30/10

(52) U.S. Cl. CPC C12N 7/045; A61K 39/29; A61P 31/14; C07K 14/1833; C12Q 1/68; G16B 15/10; G16B 30/10; A61K 2039/5254; C12N 2770/24221; C12N 2770/24222; C12N 2770/24234; C12N 2770/24262; C12N 2770/24271

(57) ABSTRACT

Attenuated forms of virulent Zika virus and Hepatitis C virus are provided. Vaccine compositions comprising the attenuated virus and methods of eliciting a protective immune response in a subject by administering the vaccine compositions are also provided.

### CROSS REFERENCE TO RELATED APPLICATIONS

This application is a continuation-in-part of International Patent Application PCT/IL2023/050857, filed Aug. 15, 2023, and also claims the benefit of priority of U.S. Provisional Patent Application No. 63/878,149, filed Sep. 9, 2025, the contents of which are all incorporated herein by reference in their entirety.

### REFERENCE TO AN ELECTRONIC SEQUENCE LISTING

The contents of the electronic sequence listing (BIU-P-051-US SQL.xml; Size: 138,877 bytes; and Date of Creation: Feb. 16, 2026) is herein incorporated by reference in its entirety.

### FIELD OF INVENTION

The present invention is in the field of virus attenuation and vaccine production.

### BACKGROUND OF THE INVENTION

Viruses have always been one of the main causes of death and disease in man. Unlike bacterial diseases, viral diseases are not susceptible to antibiotics and are thus difficult to treat. Accordingly, vaccination has been humankind's main and most robust defense against viruses. Today, some of the oldest and most serious viral diseases such as smallpox and poliomyelitis (polio) have been eradicated (or nearly so) by world-wide programs of immunization. However, many other old viruses such as rhinovirus and influenza virus are poorly controlled, and still create substantial problems, though these problems vary from year to year and country to country. In addition, relatively newer viruses, such as Human Immunodeficiency Virus (HIV) and Severe Acute Respiratory Syndrome (SARS) virus, regularly appear in human populations and often cause deadly pandemics. There is also a potential for lethal man-made or man-altered viruses for intentional introduction as a means of warfare or terrorism.

An attenuated live vaccine comprises a virus that has been subjected to mutations rendering it to a less virulent and usable for immunization. Live, attenuated viruses have many advantages as vaccines: they are often easy, fast, and cheap to manufacture; they are often easy to administer (the Sabin polio vaccine, for instance, was administered orally on sugar cubes); and sometimes the residual growth of the attenuated virus allows “herd” immunization (immunization of people in close contact with the primary patient). These advantages are particularly important in an emergency, when a vaccine is rapidly needed. The major drawback of an attenuated vaccine is that it has some significant frequency/probability of reversion to wild type virulence. For example, for this reason, the Sabin vaccine is no longer used in the United States.

To overcome the numerous pitfalls attributed to the classical vaccine design strategies, more efficient and robust rational approaches based on computer-based methods are highly desirable. One direction in designing in-silico vaccine candidates may be based on exploiting the synonymous information encoded in the genomes for attenuating the viral replication cycle while retaining the wild type proteins.

PCT Application No. WO2017056094 discloses a method for obtaining an attenuated virus with reduced replicative fitness as compared to the wild-type virus that contains an RNA, or DNA encoding the RNA. The method relies on at least one synonymous substitution in a region of evolutionarily conserved local RNA folding energy, where the substitution increases folding energy in a region with local folding energy below a predetermined threshold, or decreases folding energy in a region with local folding energy above a predetermined threshold, and where the threshold is derived from the average local folding energy of a randomized sequence.

Zika virus (or ZIKV), a member of the genus Flavivirus in the family Flaviviridae, is an enveloped, single-stranded positive-sense RNA virus. The full-length ZIKV genome sequence contains 10,794 nucleotides encoding 3,419 amino acids, two flanking untranslated regions (5′ and 3′ UTRs), and a single long open reading frame that encodes a polyprotein, which is cleaved into the capsid (C), precursor membrane (prM), envelope (E) and seven nonstructural (NS) proteins (5′-C-prM-E-NS1-NS2A-NS2B-NS3-NS4A-NS4B-NS5-3′). Zika virus is spread by daytime-active Aedes mosquitoes, such as A. aegypti and A. albopictus. Since the 1950s, it has been known to occur within a narrow equatorial belt from Africa to Asia. From 2007 to 2016, the virus spread eastward, across the Pacific Ocean to the Americas, leading to the 2015-2016 Zika virus epidemic. The infection often causes no or only mild symptoms, however, Zika can spread from a pregnant woman to her baby, which can result in microcephaly, severe brain malformations, and other birth defects. Moreover, Zika infections in adults may result rarely in Guillain-Barré syndrome.

Relevant background art includes PCT Application No. WO 2008121992 and Synthetic Biology: Advances in Molecular Biology and Medicine, edited by Robert Allen Meyers, pages 590-618, 2015.

### SUMMARY OF THE INVENTION

The present invention provides modified genomes of an organism comprising at least one coding sequence comprising at least one mutation, wherein the mutation generates an underrepresented sequence that is underrepresented in the unmodified genome of the organism. Organisms and cells comprising the modified genomes of the invention, as well as methods of making the modified genomes are also provided.

According to a first aspect, there is provided an attenuated form of a virulent virus, comprising a Zika virus genome comprising at least two regions of synonymous codon substitution, wherein a first region encodes a NS5 protein and the synonymous codon substitution comprises synonymous substitutions that deoptimize local folding energy within the first region and wherein a second region encodes at least one Zika virus structural protein selected from the E protein, the C protein and the prM protein and the synonymous codon substitution comprises synonymous substitutions that replace a more common codon within the Zika virus with a less common codon.

According to some embodiments, the less common codon is a codon that is used in at least 10% of Zika viruses at a position in which a synonymous substitution is being generated.

According to some embodiments, the less common codon is a codon that is used in at least 15% of Zika viruses at a position in which a synonymous substitution is being generated.

According to some embodiments, the deoptimizing comprises increasing the local folding energy in a region of evolutionarily conserved strong folding, decreasing the local folding energy in a region of evolutionarily conserved weak folding, or both.

According to some embodiments, first region before synonymous substitution comprises SEQ ID NO: 1.

According to some embodiments, the synonymous substitutions within SEQ ID NO: 1 comprise substitutions at at least the group of codons consisting of 77, 78, 80, 81, 82, 83, 86, 88, 90, 91, 92, 93, 94, 136, 137, 139, 140, 141, 142, 143, 144, 146, 148, 150, 173, 174, 175, 177, 178, 180, 184, 186, 187, 189, 232, 233, 234, 235, 236, 237, 238, 239, 240, 241, 242, 243, 246, 247, 248, 249, 251, 253, 312, 313, 314, 316, 318, 319, 322, 324, 326, 327, 329, 330, 331, 336, 337, 338, 340, 341, 343, 346, 348, 497, 500, 502, 503, 504, 506, 507, 509, 511, 512, 513, 514, 516, 520, 521, 524, 525, 526, 527, 529, 530, 615, 616, 617, 618, 620, 621, 622, 623, 624, 626, 629, 631, 632, 635, 639, 640, 641, 643, 666, 668, 669, 670, 671, 672, 673, 675, 676, 678, 679, 681, 682, 683, 684, 685, 690, 691, 692, 693, 697, 698, 699, 700, 703, 708, 709, 712, 828, 830, 831, 832, 833, 834, 836, 838, 842, 843, 844, 849, 850, 851, 852, 853, 870, 872, 874, 877, 878, 879, 880, 881, 886, 887, 888, 889, and 890.

According to some embodiments, the synonymous substitutions within SEQ ID NO: 1 comprise the group of mutations consisting of: C231G, T234C, T240G, A243G, T246C, C249G, C258A, A262T, G263C, C264A, C270T, C273A, C276T, C279T, C282A, G408C, T411C, G417A, T420C, C423T, G426T, T427C, G429C, G432A, T438C, T444A, T448A, C449G, A450C, A519G, A522G, A525G, A531G, C534A, T540C, T550C, A558C, C561T, A565T, G566C, C567T, A694T, G695C, T696A, G699T, C702A, C705A, G708A, A709T, G710C, C711A, G714A, C717A, C720A, T721C, G723C, G726A, C727A, C729A, G738A, C741T, A742C, G744C, G747A, G753T, T759C, C936A, T939C, A942T, G948C, G954A, T955A, C956G, A966T, G972C, C978T, G981A, G987A, T988A, C989G, A990T, A993G, G1008T, T1011A, A1014G, A1020T, A1023C, T1029C, C1038T, A1044T, C1491T, G1500A, G1506A, C1509T, T1510A, C1511G, A1512C, T1518A, T1521G, A1527G, G1533C, A1536C, T1537C, A1539C, A1542G, C1548G, A1560C, A1563G, A1570T, G1571C, T1572C, C1575G, A1578C, A1581T, A1587C, A1588C, C1845T, C1848T, A1851C, G1854T, G1860A, C1863T, T1866A, C1867A, G1869A, T1872C, G1878A, A1887G, T1891C, G1893A, G1896A, C1905T, C1915T, C1918A, G1923A, G1929A, T1998C, T2004G, G2007T, G2010A, A2013T, T2016A, T2019C, A2023C, G2025C, C2028T, T2034C, C2037T, A2041C, G2043C, C2046T, T2047C, T2052C, T2055C, A2068C, G2073A, C2076T, A2079T, A2091G, C2094T, A2097T, A2100C, C2109T, T2124G, G2127A, T2134A, C2135G, C2484T, C2490T, A2493C, T2496C, G2499C, A2502G, A2508C, T2514C, A2526G, A2529G, A2530C, T2547C, A2550G, T2551A, C2552G, C2556T, A2559C, C2610T, G2616C, A2620C, G2622C, T2631G, T2634C, A2637G, A2640G, A2643G, A2658G, T2659A, C2660G, C2661T, C2664T, A2667G, and G2670T.

According to some embodiments, the first region after synonymous substitution comprises SEQ ID NO: 12.

According to some embodiments, the second region before synonymous substitution comprises SEQ ID NO: 15.

According to some embodiments, the synonymous substitutions within SEQ ID NO: 15 comprise substitutions at at least the group of codons consisting of
a. 5, 24, 42, 47, 53, 57, 64, 71, 88, 92, 104, 107, 112, 138, 147, 154, 163, 164, 189, 194, 203, 216, 221, 222, 224, 233, 247, 258, 264, 265, 276, 299, 304, 319, 329, 330, 339, 344, 377, 378, 385, 394, 403, 410, 422, 431, 433, 442, 456, 468, 476, 482, 497, 503, 511, 516, 532, 538, 541, 550, 554, 558, 583, 595, 613, 614, 622, 628, 640, 648, 653, 654, 662, 667, and 674; or
b. 28, 33, 49, 67, 118, 128, 137, 141, 146, 152, 155, 157, 172, 174, 181, 205, 227, 235, 241, 244, 255, 259, 284, 294, 303, 313, 321, 323, 338, 341, 351, 362, 386, 393, 411, 414, 416, 424, 425, 439, 451, 453, 461, 470, 472, 479, 491, 494, 498, 505, 521, 533, 544, 548, 563, 569, 578, 600, 608, 633, 661, 668, 681, 688, 694, 699, 702, 714, 721, 728, 731, 735, 736, 762, and 781.

According to some embodiments, the synonymous substitutions within SEQ ID NO: 15 comprise the group of mutations consisting of:
a. A15G, G72A, G126A, C141T, T159C, G171A, T192C, A213C, G264T, C274T, A312G, T321C, T336C, A414G, T441C, T462C, T489C, A492G, T567C, G582A, C609T, T648C, C663T, T666C, T672A, C699T, C741T, G774T, C792T, T795C, C828T, G897A, T912C, T957C, G987A, T990A, A1017G, G1032C, C1131T, T1134C, G1155A, A1182T, C1207T, A1230T, A1266G, C1291T, T1299C, C1326T, G1368A, C1404A, C1426T, A1428G, G1446A, T1491C, T1509G, T1533C, C1548T, C1594T, A1614C, A1623G, T1650C, A1662C, C1674T, G1749A, C1785T, C1839T, G1842A, C1866T, T1884C, A1920G, T1942C, T1959G, A1962G, T1986A, A2001G, and C2022T; or
b. G84A, C97T, G147A, T201C, C354T, T384C, C411T, T423C, T438C, G456A, G465A, T471C, C516T, A522G, G543A, C615T, C679T, T703C, G705A, A723G, T730C, T765C, T775C, G777C, C850T, A882T, A909G, T939C, C963T, A969G, A1014G, C1023T, T1053C, C1086T, A1158G, T1179C, C1233T, A1242G, C1248T, T1272C, C1273T, T1317C, T1353C, T1359C, C1383T, C1408T, T1416C, T1437C, T1471C, T1480C, C1494T, G1515A, C1563T, A1599G, T1632C, A1644G, G1689A, T1707A, C1734T, A1800G, G1824A, T1899C, C1983T, T2004C, C2043T, C2064T, C2082T, A2097G, A2106G, T2140C, T2163C, C2184T, T2191C, C2205T, T2208C, G2286A, T2341C, and A2343G.

According to some embodiments, the second region after synonymous substitution comprises SEQ ID NO: 17 or SEQ ID NO: 18.

According to some embodiments, the attenuated form of a virulent virus comprises SEQ ID NO: 12 and either SEQ ID NO: 17 or SEQ ID NO: 18.

According to some embodiments, the first region encoding the NS5 protein comprises SEQ ID NO: 12 and wherein the second region encoding the at least one structural protein comprises SEQ ID NO: 17 or SEQ ID NO: 18.

According to some embodiments, the first region is at least one of:
a. comprising fewer than 296 synonymous codon substitutions;
b. not comprising codon substitutions at all of the following codons in SEQ ID NO: 1: 5, 6, 8, 10, 23, 25, 29, 30, 32, 41, 43, 44, 45, 48, 51, 56, 58, 60, 61, 63, 69, 71, 73, 76, 81, 83, 84, 92, 93, 94, 102, 103, 104, 106, 116, 118, 127, 128, 130, 131, 132, 141, 147, 149, 150, 151, 153, 154, 156, 162, 163, 168, 172, 176, 178, 181, 183, 186, 187, 188, 189, 196, 198, 200, 201, 203, 204, 205, 207, 208, 209, 210, 213, 216, 218, 223, 228, 230, 232, 233, 234, 235, 239, 243, 248, 249, 250, 251, 255, 257, 259, 260, 265, 266, 268, 272, 274, 281, 285, 286, 298, 300, 302, 303, 306, 309, 312, 317, 324, 325, 328, 330, 335, 336, 337, 338, 340, 341, 342, 346, 353, 354, 356, 360, 361, 363, 364, 365, 385, 392, 396, 399, 403, 406, 408, 409, 412, 413, 416, 421, 426, 427, 430, 435, 436, 440, 443, 445, 446, 447, 452, 453, 454, 459, 461, 463, 472, 483, 490, 492, 495, 501, 504, 505, 512, 517, 528, 529, 533, 537, 538, 541, 542, 549, 556, 561, 562, 572, 573, 574, 578, 580, 583, 584, 585, 590, 593, 597, 598, 599, 602, 604, 605, 606, 608, 610, 613, 615, 616, 618, 619, 623, 627, 628, 632, 643, 646, 653, 657, 663, 668, 671, 675, 676, 677, 679, 680, 681, 683, 689, 692, 693, 698, 700, 701, 715, 716, 717, 720, 721, 723, 725, 727, 731, 732, 733, 745, 747, 749, 750, 751, 752, 753, 757, 759, 767, 769, 771, 773, 776, 780, 785, 786, 788, 791, 792, 793, 794, 796, 798, 799, 801, 802, 803, 810, 814, 817, 818, 821, 822, 823, 828, 829, 836, 837, 839, 844, 846, 850, 853, 854, 856, 858, 862, 864, 868, 869, 875, 876, 886, 887, 891, 892, 897, 899, and 901; and
c. not comprising SEQ ID NO: 14.

According to another aspect, there is provided an attenuated form of a virulent virus, comprising a Zika virus genome comprising at least one region of synonymous codon substitution,
a. wherein the region encodes a NS5 protein and before the synonymous codon substitution comprises SEQ ID NO: 1, and wherein the synonymous substitutions within SEQ ID NO: 1 comprise substitutions at least the group of codons consisting of:
   i. 1, 17, 59, 79, 129, 158, 179, 227, 258, 276, 290, 310, 322, 349, 374, 422, 448, 469, 487, 516, 536, 559, 576, 595, 636, 655, 694, 718, 743, 809, 833, and 865;
   ii. 3, 11, 33, 38, 46, 50, 62, 70, 77, 80, 90, 98, 105, 124, 133, 143, 148, 159, 164, 170, 180, 190, 206, 215, 225, 241, 246, 261, 271, 278, 288, 292, 296, 305, 311, 316, 320, 326, 332, 350, 355, 366, 384, 389, 395, 404, 411, 423, 434, 439, 464, 467, 470, 480, 484, 496, 502, 511, 518, 524, 530, 540, 553, 560, 565, 570, 579, 591, 603, 611, 617, 622, 630, 638, 649, 656, 670, 687, 695, 704, 710, 719, 728, 740, 746, 756, 778, 807, 812, 825, 831, 841, 851, 860, 867, 885, and 898;
   iii. 136, 137, 139, 142, 143, 146, 147, 148, 149, 150, 312, 313, 314, 316, 317, 318, 319, 320, 321, 322, 323, 324, 325, 326, 327, 328, 329, 330, and 332;
   iv. 77, 79, 80, 81, 83, 84, 85, 86, 88, 89, 92, 93, 94, 136, 137, 139, 142, 143, 146, 147, 148, 149, 150, 312, 313, 314, 316, 317, 318, 319, 320, 321, 322, 323, 324, 325, 326, 327, 328, 329, 330, 332, 337, 338, 339, 342, 343, 345, 615, 617, 618, 619, 621, 622, 623, 624, 626, 627, 628, 631, 632, 636, 638, 639, 642, 643, 644, 645, 669, 670, 671, 672, 673, 675, 678, 680, 681, 683, 684, 687, 689, 690, 692, 695, 699, 701, 703, 706, 708, 709, 710, 712, 713, 714, 870, 872, 873, 874, 877, 878, 881, 884, and 886;
   v. 77, 78, 80, 83, 85, 86, 88, 89, 90, 91, 92, 93, 94, 136, 137, 139, 142, 143, 144, 146, 148, 150, 312, 313, 314, 316, 317, 318, 319, 321, 322, 324, 327, 328, 329, 330, 332, 337, 338, 340, 341, 342, 343, 615, 618, 619, 620, 622, 623, 624, 626, 627, 628, 631, 632, 636, 638, 639, 640, 641, 642, 643, 645, 667, 669, 670, 671, 672, 674, 675, 676, 678, 679, 680, 681, 682, 685, 690, 691, 695, 698, 699, 701, 703, 704, 708, 709, 711, 712, 713, 714, 872, 873, 874, 875, 876, 877, 878, 879, 880, 881, 884, 885, 886, and 887;
   vi. 1, 17, 59, 77, 78, 79, 80, 81, 82, 83, 86, 88, 90, 91, 92, 93, 94, 129, 136, 137, 139, 140, 141, 142, 143, 144, 146, 148, 150, 158, 179, 227, 258, 276, 290, 310, 312, 313, 314, 316, 318, 319, 322, 324, 326, 327, 329, 330, 331, 336, 337, 338, 340, 341, 343, 346, 348, 349, 374, 422, 448, 469, 487, 516, 536, 559, 576, 595, 615, 616, 617, 618, 620, 621, 622, 623, 624, 626, 629, 631, 632, 635, 636, 639, 640, 641, 643, 655, 666, 668, 669, 670, 671, 672, 673, 675, 676, 678, 679, 681, 682, 683, 684, 685, 690, 691, 692, 693, 694, 697, 698, 699, 700, 703, 708, 709, 712, 718, 743, 809, 833, 865, 870, 872, 874, 877, 878, 879, 880, 881, 886, 887, 888, 889, and 890;
   vii. 1, 7, 17, 49, 59, 68, 79, 89, 108, 129, 142, 158, 161, 179, 184, 227, 237, 258, 263, 276, 290, 310, 314, 322, 331, 349, 352, 374, 382, 402, 422, 432, 448, 462, 469, 477, 487, 500, 516, 522, 536, 544, 559, 564, 576, 582, 595, 601, 636, 640, 655, 666, 694, 718, 724, 743, 755, 809, 816, 833, 847, and 865; or
   viii. 3, 4, 7, 8, 15, 16, 22, 43, 44, 47, 57, 65, 68, 84, 116, 125, 126, 142, 143, 161, 163, 164, 168, 172, 195, 197, 198, 205, 208, 211, 222, 262, 263, 267, 303, 320, 325, 336, 338, 339, 354, 355, 370, 378, 382, 393, 394, 410, 413, 434, 435, 445, 480, 485, 489, 507, 508, 511, 512, 513, 515, 520, 548, 552, 553, 564, 565, 567, 579, 593, 606, 607, 610, 617, 618, 631, 638, 649, 668, 669, 679, 680, 741, 742, 775, 776, 790, 794, 810, 812, 813, 817, 836, 837, 851, 858, 859, 872, and 901;
b. wherein the region encodes a structural protein selected from the E protein, the C protein and the prM protein and before the synonymous codon substitution comprises SEQ ID NO: 15, and wherein the synonymous substitutions within SEQ ID NO: 15 comprise substitutions at at least the group of codons consisting of:
   ix. 5, 24, 42, 47, 53, 57, 64, 71, 88, 92, 104, 107, 112, 138, 147, 154, 163, 164, 189, 194, 203, 216, 221, 222, 224, 233, 247, 258, 264, 265, 276, 299, 304, 319, 329, 330, 339, 344, 377, 378, 385, 394, 403, 410, 422, 431, 433, 442, 456, 468, 476, 482, 497, 503, 511, 516, 532, 538, 541, 550, 554, 558, 583, 595, 613, 614, 622, 628, 640, 648, 653, 654, 662, 667, and 674;
   x. 28, 33, 49, 67, 118, 128, 137, 141, 146, 152, 155, 157, 172, 174, 181, 205, 227, 235, 241, 244, 255, 259, 284, 294, 303, 313, 321, 323, 338, 341, 351, 362, 386, 393, 411, 414, 416, 424, 425, 439, 451, 453, 461, 470, 472, 479, 491, 494, 498, 505, 521, 533, 544, 548, 563, 569, 578, 600, 608, 633, 661, 668, 681, 688, 694, 699, 702, 714, 721, 728, 731, 735, 736, 762, and 781;
   xi. 108, 110, 112, 113, 114, 116, 118, 119, 120, 123, 252, 253, 255, 256, 257, 258, 260, 262, 263, 265, 268, 269, 270, 370, 371, 373, 376, 378, 381, 541, 542, 543, 545, 546, 547, 548, 549, 550, 551, 555, 557, 558, 560, 704, 706, 707, 708, 712, 713, 714, 715, 716, 717, 718, 739, 740, 741, 742, 743, 746, 747, 749, 751, 753, 759, 760, 761, 766, 767, 768, 769, 770, 771, 772, and 773;
   xii. 109, 110, 111, 112, 113, 114, 115, 116, 119, 120, 122, 123, 252, 255, 256, 257, 258, 259, 260, 261, 263, 264, 267, 268, 270, 369, 370, 372, 374, 375, 376, 377, 378, 379, 381, 382, 542, 543, 545, 546, 547, 548, 549, 550, 552, 554, 555, 557, 559, 560, 704, 705, 707, 708, 709, 713, 718, 742, 743, 746, 747, 748, 749, 751, 759, 760, 761, 762, 765, 766, 767, 769, 771, 772, and 773; or
   xiii. 19, 29, 30, 33, 36, 47, 62, 64, 104, 114, 128, 136, 178, 182, 198, 201, 202, 218, 235, 242, 246, 247, 251, 252, 258, 267, 280, 295, 296, 311, 313, 314, 315, 332, 336, 357, 372, 374, 384, 385, 386, 387, 389, 396, 404, 425, 469, 477, 484, 485, 499, 502, 510, 522, 545, 546, 558, 563, 574, 583, 589, 590, 592, 596, 615, 625, 638, 641, 648, 666, 668, 680, 681, 727, 730, 731, 738, 745, 746, 759, 760, 765, 766, 775, 780, 783, 787; or
c. both (a) and (b).

According to some embodiments, the synonymous substitutions comprise at least one group of mutations selected from:
a. G3A, C51T, T177C, T237C, G387A, A474G, T537C, C681T, T774C, G828A, G870A, T930C, A966G, G1047A, T1122G, T1266A, C1344T, C1407A, C1461T, C1548G, T1608C, G1677A, C1728T, T1785C, T1906C, T1965C, A2082G, C2154G, T2229A, A2427G, G2499A, and T2595C within SEQ ID NO: 1;
b. A9G, A33G, C99T, A114G, C138T, G150A, G186C, C210T, C231T, T240C, C270T, A294G, A315G, C372T, T399C, T427C, T444C, A477T, C492G, T510C, T540C, T570C, C616T, T645A, G675A, T721C, G738C, T783A, A813G, T834C, G864A, G876A, C888T, T915C, G933A, G948A, T960C, C978T, C996T, T1050C, T1065C, C1098T, A1152G, C1167T, T1185C, T1212G, G1233A, A1269T, T1302C, A1317G, G1392A, A1401G, G1410A, C1438T, A1440G, T1452C, T1488C, G1506A, C1531T, T1554C, T1572C, G1590A, C1620T, A1659G, A1680G, T1693C, C1710T, A1737G, T1773G, C1809T, T1833C, C1849T, A1851G, T1866C, T1890G, C1912T, T1945C, G1968A, G2010A, A2061G, G2085A, C2112T, T2130C, T2157C, T2184C, C2220T, G2238A, A2268G, C2334T, C2421T, T2436C, C2475T, A2493T, T2521C, T2553C, C2580T, C2601T, C2655T, and T2694C within SEQ ID NO: 1;
c. G408C, T411C, G417T, G426T, T427C, G429A, T438C, A441T, T444C, G447A, A450C, C936T, T939A, A942T, G948A, T949A, C950G, A951C, G954T, C957A, T960A, G963T, A966T, C969T, G972C, T975G, C978G, A979C, G981C, C982T, C984G, G987T, A990C, and C996A within SEQ ID NO: 1;
d. C231G, T237C, C238T, T240G, A243C, C249G, A250C, A252G, G255A, C258G, A262T, G263C, C267T, C276T, C279A, C282T, G408C, T411C, G417T, G426T, T427C, G429A, T438C, A441T, T444C, G447A, A450C, C936T, T939A, A942T, G948A, T949A, C950G, A951C, G954T, C957A, T960A, G963T, A966T, C969T, G972C, T975G, C978G, A979C, G981C, C982T, C984G, G987T, A990C, C996A, T1011A, A1014C, C1017T, A1026T, T1029A, C1035T, C1845A, A1851C, G1854C, G1857C, C1863G, T1866A, C1867A, T1872C, G1878A, T1881C, G1884A, T1891C, G1896A, T1906C, G1914C, G1917A, A1926T, G1929A, A1932G, G1935C, G2007C, G2010A, A2013C, T2016C, T2019C, A2023C, T2034C, C2038T, C2040G, A2041C, G2043C, T2047C, G2049T, T2052C, A2061C, T2067C, A2068C, C2076T, G2085A, T2095A, C2096G, A2097T, A2103C, C2109T, A2118G, T2124G, G2127T, T2130C, C2136A, C2139T, T2142C, C2610T, G2616T, C2619G, A2620C, G2622C, T2631A, T2634C, A2643G, C2652T, and A2658G within SEQ ID NO: 1;
e. C231T, T234A, C238T, T240G, C249A, G255A, C258A, C264T, C267T, C270T, C273A, C276A, C279A, C282A, G408T, T411A, G417A, G426T, T427C, G429C, G432C, T438C, T444G, T448A, C449G, A450T, C936A, T939A, A942T, G948A, A951C, G954A, C957A, G963C, A966T, G972C, G981A, C984A, G987A, T988A, C989G, A990T, C996A, T1011A, A1014G, A1020T, A1023C, A1026T, T1029C, C1845A, G1854T, G1857T, G1860A, T1866A, C1867A, G1869A, T1872C, G1878A, T1881A, G1884A, T1891C, G1893A, G1896A, T1906C, G1908A, G1914A, G1917C, C1918A, G1920A, G1923A, A1926C, G1929A, G1935T, C2001T, G2007T, G2010A, A2013T, T2016A, T2022C, A2023C, G2025C, C2028T, T2034C, C2037T, C2038T, C2040G, G2043A, C2046T, T2055C, A2068C, G2070C, G2073A, G2085A, C2094A, T2095A, C2096G, A2097T, A2103C, C2109T, C2112T, T2124G, G2127A, C2133T, C2136A, C2139T, T2142C, G2616T, C2617A, C2619A, A2620C, C2625A, A2628C, T2631C, T2634C, A2637G, A2640G, A2643G, C2652T, C2655T, A2658G, and C2661T within SEQ ID NO: 1;
f. G3A, C51T, T177C, C231G, T234C, T237C, T240G, A243G, T246C, C249G, C258A, A262T, G263C, C264A, C270T, C273A, C276T, C279T, C282A, G387A, G408C, T411C, G417A, T420C, C423T, G426T, T427C, G429C, G432A, T438C, T444A, T448A, C449G, A450C, A474G, T537C, C681T, T774C, G828A, G870A, T930C, C936A, T939C, A942T, G948C, G954A, T955A, C956G, A966G, G972C, C978T, G981A, G987A, T988A, C989G, A990T, A993G, G1008T, T1011A, A1014G, A1020T, A1023C, T1029C, C1038T, A1044T, G1047A, T1122G, T1266A, C1344T, C1407A, C1461T, C1548G, T1608C, G1677A, C1728T, T1785C, C1845T, C1848T, A1851C, G1854T, G1860A, C1863T, T1866A, C1867A, G1869A, T1872C, G1878A, A1887G, T1891C, G1893A, G1896A, C1905T, T1906C, C1915T, C1918A, G1923A, G1929A, T1965C, T1998C, T2004G, G2007T, G2010A, A2013T, T2016A, T2019C, A2023C, G2025C, C2028T, T2034C, C2037T, A2041C, G2043C, C2046T, T2047C, T2052C, T2055C, A2068C, G2073A, C2076T, A2079T, A2082G, A2091G, C2094T, A2097T, A2100C, C2109T, T2124G, G2127A, T2134A, C2135G, C2154G, T2229A, A2427G, G2499A, T2595C, C2610T, G2616C, A2620C, G2622C, T2631G, T2634C, A2637G, A2640G, A2643G, A2658G, T2659A, C2660G, C2661T, C2664T, A2667G, and G2670T within SEQ ID NO: 1;
g. G3A, C21T, C51T, A147C, T177C, C202A, G204A, T237C, C267T, T324C, G387A, G426T, A474G, G483A, T537C, T550C, C681T, C711T, T774C, G789A, G828A, G870A, T930C, A942C, A966G, A993G, G1047A, G1056A, T1122G, T1144C, C1206T, T1266A, C1296T, C1344T, A1386G, C1407A, T1431C, C1461T, G1500A, C1548G, G1566A, T1608C, C1632T, G1677A, C1692T, C1728T, T1746C, T1785C, G1803A, T1906C, C1918A, T1965C, T1998C, A2082G, C2154G, G2172A, T2229A, C2265T, A2427G, C2448T, G2499A, T2539C, and T2595C within SEQ ID NO: 1;
h. G3T, C51T, A175T, G176C, T177G, T237C, G387T, A474G, T537C, A679T, G680C, C681G, T774C, G828A, G870A, T930C, A966G, G1047A, T1122A, T1266G, C1344T, C1407G, C1461T, C1548A, T1608G, G1677A, C1728T, T1785C, T1906C, G1908A, T1965C, A2082G, C2154A, T2229G, A2427G, G2499A, and T2595C within SEQ ID NO: 1;
i. A9G, A12T, C21A, G24T, C45G, G48C, G66C, C129A, C132T, T141G, C169A, A171G, T193C, G195C, C202A, A252G, G348T, T375G, T378C, G426A, T427C, G429T, G483T, A489G, C492G, G504T, T516C, G585C, A591G, G594C, A615G, A624G, C633T, C666G, C786G, G789T, A801T, A909T, T958A, C959G, T960C, T975G, G1008T, A1014G, C1017G, A1062G, T1065G, C1110G, C1134T, T1144C, G1146C, A1179G, C1182G, T1228C, A1230T, A1239C, T1302A, A1305T, A1335G, A1440C, A1455C, C1467A, T1521G, T1524G, G1533C, A1536G, T1537C, A1545G, A1560C, G1644C, T1656A, A1659T, C1692A, T1693C, G1695T, T1699C, G1701T, A1737T, C1779T, T1818G, C1821T, T1830A, A1851G, G1854T, T1891C, G1893C, G1914C, T1945C, G1947C, T2004G, G2007T, C2037A, C2040T, C2223G, C2226G, A2325G, G2328C, T2370G, A2382G, C2430T, T2436G, G2439T, A2451G, A2508T, C2511T, T2551A, C2552G, T2553C, C2574G, C2577T, G2616T, and A2703G within SEQ ID NO: 1;
j. A15G, G72A, G126A, C141T, T159C, G171A, T192C, A213C, G264T, C274T, A312G, T321C, T336C, A414G, T441C, T462C, T489C, A492G, T567C, G582A, C609T, T648C, C663T, T666C, T672A, C699T, C741T, G774T, C792T, T795C, C828T, G897A, T912C, T957C, G987A, T990A, A1017G, G1032C, C1131T, T1134C, G1155A, A1182T, C1207T, A1230T, A1266G, C1291T, T1299C, C1326T, G1368A, C1404A, C1426T, A1428G, G1446A, T1491C, T1509G, T1533C, C1548T, C1594T, A1614C, A1623G, T1650C, A1662C, C1674T, G1749A, C1785T, C1839T, G1842A, C1866T, T1884C, A1920G, T1942C, T1959G, A1962G, T1986A, A2001G, and C2022T within SEQ ID NO: 15;
k. G84A, C97T, G147A, T201C, C354T, T384C, C411T, T423C, T438C, G456A, G465A, T471C, C516T, A522G, G543A, C615T, C679T, T703C, G705A, A723G, T730C, T765C, T775C, G777C, C850T, A882T, A909G, T939C, C963T, A969G, A1014G, C1023T, T1053C, C1086T, A1158G, T1179C, C1233T, A1242G, C1248T, T1272C, C1273T, T1317C, T1353C, T1359C, C1383T, C1408T, T1416C, T1437C, T1471C, T1480C, C1494T, G1515A, C1563T, A1599G, T1632C, A1644G, G1689A, T1707A, C1734T, A1800G, G1824A, T1899C, C1983T, T2004C, C2043T, C2064T, C2082T, A2097G, A2106G, T2140C, T2163C, C2184T, T2191C, C2205T, T2208C, G2286A, T2341C, and A2343G within SEQ ID NO: 15;
l. T324C, C330G, T336C, T339C, C342G, A348C, C354A, A357C, C360A, G369C, C756T, G759A, T765A, C768G, C771T, G774T, A780C, T786C, C789T, T795A, T802C, G804A, A807C, A808T, G809C, C810A, C1110T, C1113T, C1119T, T1126A, C1127G, A1128T, T1134A, C1143G, A1623G, A1624C, A1629G, C1635G, A1638C, T1641G, A1644C, G1647C, A1648T, G1649C, A1653G, T1665G, G1671C, C1674A, T1680A, T2112A, A2116C, T2121A, C2124T, A2136C, C2139G, T2140C, G2142T, A2145G, C2148T, A2151T, C2154T, T2217C, A2220C, A2223C, C2226T, C2229T, T2236C, G2238T, T2241C, A2247G, C2253T, C2259T, A2277G, G2280C, T2281C, G2283C, T2298G, C2299T, T2304C, A2307T, G2310A, T2313C, A2316G, and T2319C within SEQ ID NO: 15;
m. A325T, G326C, T327C, C330G, A333C, T336A, T339G, C342G, C343T, C345G, A348C, A357C, C360T, A366C, G369C, C756T, T765C, C768A, C771T, G774T, T775C, G777C, A780T, A783T, C789T, C792T, T801A, T802C, G804T, C810T, A1107G, C1110T, T1116C, G1122A, A1125G, A1128T, C1131T, T1134C, A1137G, C1143T, C1146T, A1624C, G1626C, A1629G, C1635G, A1638G, T1641G, A1644C, G1647C, A1648T, G1649C, T1650C, A1656G, A1662C, T1665G, G1671C, T1677G, T1680C, T2112A, G2115T, T2121A, C2124A, G2127A, C2139T, C2154T, C2226A, C2229T, T2236C, G2238T, T2241C, A2244C, A2247C, C2253T, A2277G, G2280C, T2281C, G2283T, G2286T, T2293C, G2295A, T2298C, G2301C, A2307T, T2313C, A2316G, and T2319C within SEQ ID NO: 15; and
n. C55A, C57G, C87G, T88C, G99C, A108G, C141T, T184A, C185G, A186C, T192G, C310A, A312G, C342G, C382A, T384G, T406C, G408C, G534C, A546G, T594G, A603G, C606T, G654A, T703C, G705C, G726A, A738G, C741G, A753C, C756T, G774A, T801C, C840T, A885G, C888G, T933G, T939G, C942T, T943C, G945C, C996T, C1008T, C1071T, T1116C, G1122A, A1152G, G1155T, A1158G, G1161T, A1167G, A1188G, G1212T, G1275C, C1407A, A1431G, A1452T, C1455G, G1497A, T1504C, G1506C, C1530T, A1566G, C1635G, A1638T, C1674A, G1689C, G1722C, G1749C, A1767G, T1768C, G1770C, C1776G, T1786A, C1787G, A1845T, A1875T, C1914T, T1923A, T1942C, G1944C, G1998C, T2004C, A2040G, C2043G, T2181A, T2188A, C2189G, A2190C, T2191C, T2214C, T2233A, C2234G, A2235C, T2236C, A2277G, G2280T, T2293C, G2295C, T2298G, T2323A, C2324G, C2340A, A2349G, and C2361T within SEQ ID NO: 15.

According to some embodiments, the attenuated form of a virulent virus comprises at least one of SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 13, SEQ ID NO: 17, SEQ ID NO: 18, SEQ ID NO: 19, SEQ ID NO: 20 and SEQ ID NO: 21.

According to some embodiments, the region encodes a NS5 protein and comprises a sequence selected from SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, and SEQ ID NO: 13 or encodes an E protein, a C protein and a prM protein and comprises a sequence selected from SEQ ID NO: 17, SEQ ID NO: 18, SEQ ID NO: 19, SEQ ID NO: 20 and SEQ ID NO: 21.

According to some embodiments, the region is at least one of:
a. comprising fewer than 296 synonymous codon substitutions;
b. not comprising codon substitutions at all of the following codons in SEQ ID NO: 5, 6, 8, 10, 23, 25, 29, 30, 32, 41, 43, 44, 45, 48, 51, 56, 58, 60, 61, 63, 69, 71, 73, 76, 81, 83, 84, 92, 93, 94, 102, 103, 104, 106, 116, 118, 127, 128, 130, 131, 132, 141, 147, 149, 150, 151, 153, 154, 156, 162, 163, 168, 172, 176, 178, 181, 183, 186, 187, 188, 189, 196, 198, 200, 201, 203, 204, 205, 207, 208, 209, 210, 213, 216, 218, 223, 228, 230, 232, 233, 234, 235, 239, 243, 248, 249, 250, 251, 255, 257, 259, 260, 265, 266, 268, 272, 274, 281, 285, 286, 298, 300, 302, 303, 306, 309, 312, 317, 324, 325, 328, 330, 335, 336, 337, 338, 340, 341, 342, 346, 353, 354, 356, 360, 361, 363, 364, 365, 385, 392, 396, 399, 403, 406, 408, 409, 412, 413, 416, 421, 426, 427, 430, 435, 436, 440, 443, 445, 446, 447, 452, 453, 454, 459, 461, 463, 472, 483, 490, 492, 495, 501, 504, 505, 512, 517, 528, 529, 533, 537, 538, 541, 542, 549, 556, 561, 562, 572, 573, 574, 578, 580, 583, 584, 585, 590, 593, 597, 598, 599, 602, 604, 605, 606, 608, 610, 613, 615, 616, 618, 619, 623, 627, 628, 632, 643, 646, 653, 657, 663, 668, 671, 675, 676, 677, 679, 680, 681, 683, 689, 692, 693, 698, 700, 701, 715, 716, 717, 720, 721, 723, 725, 727, 731, 732, 733, 745, 747, 749, 750, 751, 752, 753, 757, 759, 767, 769, 771, 773, 776, 780, 785, 786, 788, 791, 792, 793, 794, 796, 798, 799, 801, 802, 803, 810, 814, 817, 818, 821, 822, 823, 828, 829, 836, 837, 839, 844, 846, 850, 853, 854, 856, 858, 862, 864, 868, 869, 875, 876, 886, 887, 891, 892, 897, 899, and 901; and
c. not comprising SEQ ID NO: 14.

According to another aspect, there is provided an attenuated form of a virulent virus, comprising a Hepatitis C virus (HCV) genome comprising at least one region of synonymous codon substitution, wherein the region encodes a NS5 protein and before the synonymous codon substitution comprises SEQ ID NO: 22, and wherein the synonymous substitutions within SEQ ID NO: 22 comprise substitutions at at least the group of codons consisting of:
a. 1, 43, 106, 127, 169, 190, 211, 232, 266, 286, 327, 368, 388, 430, 468, 489, 510, 531, 552, 573, 594, 615, 657, 678, 762, 783, 804, 825, 846, 909, 951, and 993;
b. 9, 16, 23, 30, 37, 44, 51, 58, 65, 79, 86, 100, 107, 114, 121, 135, 142, 149, 156, 163, 170, 198, 205, 212, 233, 243, 250, 253, 260, 267, 280, 300, 307, 314, 321, 328, 341, 348, 362, 383, 389, 396, 410, 417, 424, 431, 438, 448, 455, 469, 476, 483, 490, 497, 504, 532, 539, 546, 560, 567, 574, 581, 602, 609, 623, 630, 637, 644, 651, 665, 672, 693, 707, 721, 728, 749, 763, 770, 777, 784, 791, 798, 805, 812, 819, 826, 833, 840, 847, 854, 875, 882, 889, 896, 903, 910, 917, 931, 945, 952, 966, 973, 987, and 994;
c. 132, 133, 138, 139, 140, 141, 145, 146, 203, 204, 206, 208, 209, 213, 214, 215, 216, 217, 486, 488, 490, 491, 493, 494, 496, 497, 498, and 500;
d. 105, 106, 107, 109, 110, 112, 113, 114, 115, 116, 117, 132, 133, 138, 139, 140, 141, 145, 146, 203, 204, 206, 208, 209, 213, 214, 215, 216, 217, 333, 334, 335, 337, 338, 339, 340, 341, 342, 343, 345, 346, 347, 349, 366, 368, 371, 372, 373, 376, 377, 378, 379, 380, 486, 488, 490, 491, 493, 494, 496, 497, 498, 500, 620, 621, 625, 627, 628, 629, 631, 632, 633, 723, 727, 728, 729, 730, 733, 734, 737, 751, 753, 757, 762, 764, 766, 836, 837, 838, 839, 840, 842, 845, 846, 848, 850, 981, 983, 984, 985, 987, 989, 990, 992, and 993;
e. 105, 106, 107, 109, 110, 113, 114, 115, 116, 117, 120, 132, 133, 139, 140, 141, 143, 145, 146, 203, 204, 206, 209, 212, 213, 214, 215, 216, 217, 333, 334, 335, 337, 338, 339, 340, 341, 342, 343, 344, 345, 346, 349, 366, 368, 369, 370, 371, 372, 376, 378, 379, 380, 486, 489, 491, 492, 493, 496, 497, 498, 499, 500, 621, 625, 627, 628, 629, 630, 631, 632, 633, 725, 728, 729, 730, 731, 733, 735, 737, 751, 756, 760, 762, 763, 764, 837, 838, 839, 840, 842, 844, 845, 846, 847, 850, 983, 984, 986, 987, 989, 990, 992, 993, and 995;
f. 105, 106, 107, 109, 110, 113, 114, 115, 116, 117, 120, 132, 138, 139, 141, 143, 144, 145, 146, 203, 206, 209, 210, 211, 213, 214, 215, 216, 217, 333, 334, 335, 336, 337, 339, 340, 341, 342, 343, 344, 346, 347, 349, 368, 369, 371, 373, 374, 376, 377, 378, 379, 380, 486, 487, 492, 493, 495, 496, 497, 498, 499, 500, 621, 625, 627, 628, 629, 630, 631, 632, 633, 723, 725, 726, 728, 729, 730, 734, 737, 750, 751, 753, 760, 762, 763, 837, 838, 840, 841, 842, 843, 844, 845, 846, 850, 982, 984, 985, 986, 987, 989, 990, 992, and 995; or
g. 12, 14, 21, 25, 46, 59, 60, 91, 95, 109, 126, 163, 224, 225, 241, 242, 282, 293, 303, 319, 320, 343, 347, 349, 350, 380, 400, 407, 415, 440, 457, 460, 461, 464, 467, 469, 478, 479, 480, 507, 508, 530, 559, 560, 608, 617, 642, 655, 663, 671, 686, 725, 736, 737, 741, 777, 784, 793, 798, 820, 824, 834, 856, 870, 885, 890, 898, 902, 903, 911, 965, 975, 986, 993, and 1053.

According to some embodiments, the synonymous substitutions comprise at least one group of mutations within SEQ ID NO: 22 selected from:
a. C3T, C144G, C345G, C408T, G534A, T595C, G597T, A663C, C726G, G831C, C894T, C1026A, T1149A, T1209G, C1338T, G1458A, A1521C, C1584T, C1647G, G1710T, A1791G, T1857G, C1920T, T2055C, C2118T, A2370T, T2445A, G2517C, C2578A, G2580A, C2652A, T2856C, G2997A, and A3159T;
b. T36C, C57T, T79C, C102T, G123A, C147T, C168G, C189T, C210A, G258C, C279T, C324G, G348T, G369A, A390C, A432G, T451A, C452G, C474T, A495G, G516A, T537G, C624T, G645C, A666C, C729T, C759T, C780T, T792C, G813A, C834T, C876G, A939G, G960T, A984C, T1008C, C1029G, A1068T, C1089T, C1131T, T1194C, G1212A, T1233C, G1278T, A1299G, G1320A, T1341G, T1365A, C1395T, C1419T, T1459C, G1461T, C1482T, C1503T, A1524T, T1545C, A1564C, G1566C, C1650T, T1669C, G1671T, A1690C, A1692G, C1734T, G1764C, A1794C, C1818G, T1881C, C1900A, T1944C, C1968T, T1989A, C2010T, G2034A, C2079T, G2100A, G2163C, A2203T, G2204C, G2247A, T2268C, C2331T, A2373G, G2397A, C2424G, T2448A, A2469G, C2499T, T2518C, G2520T, A2541G, A2562G, T2583C, T2610G, C2634A, A2655C, C2682T, A2751G, C2772T, A2791C, T2814C, C2838T, A2859T, C2886G, C2931G, C2976T, C3000T, C3051T, T3078C, T3135C, and C3162T;
c. A396G, C397T, G399A, G414T, A417G, T420C, T423A, T435G, T438G, A609C, A612C, G618A, C624T, C627A, T639G, G642A, G645A, C646A, C649A, C651G, G1458A, A1464C, C1470T, T1473G, T1479C, C1482T, G1488C, T1489C, A1494C, and T1500C;
d. C315T, C318T, G321A, C327G, C330T, A334C, G339A, G342C, C345T, G348A, G351A, A396G, C397T, G399A, G414T, A417G, T420C, T423A, T435G, T438G, A609C, A612C, G618A, C624T, C627A, T639G, G642A, G645A, C646A, C649A, C651G, C999T, C1002T, T1005C, T1011C, T1014C, T1017C, C1018T, C1020G, C1023G, C1026T, C1029A, G1035A, C1038A, G1041A, T1047A, A1098G, C1102T, C1104G, G1113T, C1116T, C1119T, T1128C, C1131T, G1134A, C1137G, C1140G, G1458A, A1464C, C1470T, T1473G, T1479C, C1482T, G1488C, T1489C, A1494C, T1500C, G1860A, A1863G, C1875T, T1881G, C1884T, T1887C, C1891T, C1893G, C1896T, C1899T, T2169C, C2181T, A2184G, A2187G, G2190T, C2199T, C2202T, T2211G, A2253G, T2259C, C2271T, T2286C, A2292G, C2296T, G2508C, T2511C, G2514C, G2517C, T2518C, G2526A, C2533A, C2535A, C2536A, C2538G, C2544T, G2550C, A2943G, A2949G, G2952C, C2955T, C2961T, A2967G, T2970C, C2976T, and T2979C;
e. C315T, C318T, G321A, C327G, C330T, G339A, G342T, C345T, G348A, G351A, G360A, A396G, C397T, A417G, T420C, T423G, A429G, T435G, T438G, A609C, A612C, G618C, C627A, G636A, T639G, G642T, G645C, G648T, C649A, C651G, C999A, C1002T, T1005G, T1011C, T1014C, T1017C, C1018T, C1020G, C1023G, C1026T, C1029A, G1032A, G1035A, C1038G, T1047A, A1098G, C1104G, G1107A, A1110G, G1113C, C1116T, T1128C, G1134A, C1137G, C1140T, G1458A, C1467T, T1473C, T1474C, T1479C, G1488C, T1489C, A1494T, C1497T, T1500C, A1863G, C1875T, T1881A, C1884T, T1887A, C1890T, C1893G, C1896A, C1899G, A2173C, A2175G, A2184T, A2187G, G2190C, C2193T, C2199T, A2203T, G2204C, C2205A, T2211G, A2253G, T2268C, A2280T, T2286C, G2289A, A2292G, T2511C, G2514C, G2517C, T2518C, G2526T, C2532A, C2533A, C2535A, C2536A, C2538A, A2539C, A2541G, G2550C, A2949G, G2952T, T2958A, C2961T, C2965A, T2970C, C2976T, T2979C, and G2985A;
f. C315T, C318T, G321A, C327G, C330T, G339A, G342C, C345A, G348A, G351A, G360A, A396C, G414T, A417G, T423G, A429G, A432G, T435A, T438G, A609C, G618A, C627T, G630A, G633A, T639G, G642A, G645T, G648T, C649A, C651G, C999T, C1002T, T1005G, T1008G, T1011G, T1017C, C1018T, C1020G, C1023A, C1026T, C1029A, G1032A, C1038G, G1041A, T1047C, C1102T, C1104G, G1107A, G1113C, C1119T, G1122A, T1128C, C1131T, G1134A, C1137A, C1140A, G1458A, T1459C, T1474C, T1479C, G1485T, G1488C, T1489C, G1491A, A1494T, C1497T, T1500C, A1863G, C1875T, T1881A, C1884T, T1887C, C1890T, C1891T, C1893G, C1896A, C1899T, T2169C, A2173C, A2175G, T2178C, A2184T, A2187G, G2190C, C2202T, T2211G, G2250A, A2253G, T2259C, A2280T, T2286C, G2289A, T2511C, G2514C, T2518C, C2523T, G2526T, C2527A, G2529A, C2532A, C2533A, C2535A, C2536A, C2538A, G2550C, G2946C, G2952T, C2955T, T2958C, C2961T, A2967G, T2970C, C2976T, and G2985A; and g. T36C, C42G, T63C, T75C, G138C, C177T, C180G, T273C, G285A, C327G, G378T, C489G, A672C, T675C, C723G, C726A, A846G, A879C, C909T, G957C, G960A, C1029A, G1041A, T1047C, C1050A, C1140A, G1200C, C1221A, T1245C, G1320A, T1369A, C1370G, C1380T, T1383C, G1392C, C1401G, A1407T, T1434C, C1437A, T1440C, A1521C, A1524C, T1590C, A1677C, C1680A, T1824C, G1851A, T1926C, T1965C, T1989A, T2011C, G2013C, T2058C, A2173C, A2175G, G2208A, T2211G, T2223C, C2331G, T2352C, G2379A, C2394T, C2460A, T2472C, A2502C, T2568C, T2610A, A2655C, C2670T, T2694C, A2706G, C2709T, T2733C, G2895C, G2925C, T2958C, T2979C, and A3159C.

According to some embodiments, the attenuated form of a virulent virus comprises any one of SEQ ID NO: 24-30.

According to some embodiments, the region encoding the NS5 protein comprises any one of SEQ ID NO: 24-30.

According to some embodiments, the region is at least one of:
a. comprising fewer than 298 synonymous codon substitutions;
b. not comprising codon substitutions at all of the following codons in SEQ ID NO: 22: 10, 13, 18, 19, 35, 36, 42, 51, 57, 59, 60, 65, 67, 74, 80, 82, 89, 90, 94, 96, 100, 105, 106, 109, 113, 118, 119, 120, 136, 142, 148, 150, 154, 156, 170, 172, 175, 179, 185, 187, 192, 193, 194, 195, 201, 206, 208, 211, 212, 221, 223, 227, 228, 232, 233, 240, 243, 251, 257, 258, 259, 262, 266, 269, 271, 273, 278, 280, 291, 293, 297, 310, 315, 316, 317, 318, 330, 342, 352, 374, 408, 409, 415, 417, 418, 420, 421, 422, 424, 427, 441, 463, 465, 466, 470, 473, 474, 484, 486, 489, 494, 501, 504, 517, 521, 527, 535, 536, 538, 541, 549, 552, 553, 555, 557, 568, 569, 570, 571, 572, 573, 574, 580, 581, 584, 585, 587, 588, 591, 593, 595, 598, 603, 604, 609, 611, 617, 623, 624, 626, 628, 630, 635, 636, 637, 640, 643, 651, 659, 660, 661, 666, 677, 687, 689, 690, 691, 694, 696, 698, 699, 702, 706, 707, 709, 715, 722, 724, 726, 727, 729, 731, 734, 740, 745, 746, 748, 754, 757, 758, 761, 765, 768, 774, 776, 785, 788, 789, 792, 797, 798, 803, 805, 806, 807, 808, 810, 811, 812, 814, 815, 816, 822, 825, 827, 829, 830, 831, 833, 834, 836, 838, 841, 848, 849, 851, 853, 861, 862, 865, 868, 869, 870, 873, 876, 877, 879, 894, 895, 896, 905, 915, 918, 919, 922, 924, 925, 928, 933, 937, 939, 941, 943, 944, 945, 948, 949, 950, 951, 955, 956, 958, 959, 962, 963, 964, 967, 969, 970, 973, 975, 979, 981, 982, 987, 988, 991, 992, 995, 996, 998, 999, 1002, 1012, 1014, 1017, 1020, 1022, 1023, 1024, 1025, 1028, 1029, 1031, 1033, 1035, 1036, 1041, 1042, 1043, 1044, 1045, 1048, 1050, 1051, 1054, 1055, and 1057; and
c. not comprising SEQ ID NO: 31.

According to some embodiments, the attenuated form of a virulent virus is a mutant of a natural isolate.

According to some embodiments, the virus is a synthetic virus, comprising a nucleotide acid selected from: single strand RNA (ssRNA), (dsRNA) double strand RNA, single strand DNA (ssDNA) and double strand DNA (dsDNA).

According to some embodiments, the nucleotide acid is ssRNA.

According to another aspect, there is provided a vaccine composition comprising an attenuated form of a virulent virus of the invention and a pharmaceutically acceptable carrier, excipient or adjuvant.

According to some embodiments, the attenuated virus induces an immune response in a host animal sufficient to provide protection from the virulent virus.

According to some embodiments, protection comprises at least one of reduced infection, reduced mortality, reduced symptoms and reduced viral load.

According to another aspect, there is provided a method for eliciting a protective immune response against a virulent virus in a subject comprising administering to the subject a prophylactically effective dose of the vaccine of the invention, thereby eliciting a protective immune response in the subject.

According to some embodiments, eliciting a protective immune response comprises immunizing or vaccinating.

According to another aspect, there is provided an attenuated Hepatitis C Virus (HCV) variant, comprising: a plurality of synonymous mutations in the coding regions of the HCV genome, wherein the synonymous mutations are designed to disrupt RNA secondary structures and regulatory sequences critical for the viral life cycle; wherein the synonymous mutations preserve the amino acid sequence of the viral proteins; and wherein the attenuated HCV variant exhibits reduced viral fitness compared to the wild-type HCV.

According to some embodiments, the reduced viral fitness comprises lower RNA levels, reduced infection percentage, smaller viral spread in liver cells or a combination thereof.

According to some embodiments, the attenuated HCV variant demonstrates genomic stability over time without significant reversion to the wild-type sequence.

According to some embodiments, the synonymous mutations are introduced in regions with high selection for strong RNA folding.

According to some embodiments, the synonymous mutations result in a change in local folding energy (LFE) by at least 15%.

According to some embodiments, the synonymous mutations are introduced in regions with underrepresented sequences.

According to some embodiments, the synonymous mutations are introduced in the NS5 gene.

According to some embodiments, the NS5 gene before the synonymous mutations comprises SEQ ID NO: 22, and wherein the synonymous mutations within SEQ ID NO: 22 comprise substitutions at at least the group of codons consisting of:
a. 1, 43, 106, 127, 169, 190, 211, 232, 266, 286, 327, 368, 388, 430, 468, 489, 510, 531, 552, 573, 594, 615, 657, 678, 762, 783, 804, 825, 846, 909, 951, and 993;
b. 9, 16, 23, 30, 37, 44, 51, 58, 65, 79, 86, 100, 107, 114, 121, 135, 142, 149, 156, 163, 170, 198, 205, 212, 233, 243, 250, 253, 260, 267, 280, 300, 307, 314, 321, 328, 341, 348, 362, 383, 389, 396, 410, 417, 424, 431, 438, 448, 455, 469, 476, 483, 490, 497, 504, 532, 539, 546, 560, 567, 574, 581, 602, 609, 623, 630, 637, 644, 651, 665, 672, 693, 707, 721, 728, 749, 763, 770, 777, 784, 791, 798, 805, 812, 819, 826, 833, 840, 847, 854, 875, 882, 889, 896, 903, 910, 917, 931, 945, 952, 966, 973, 987, and 994;
c. 132, 133, 138, 139, 140, 141, 145, 146, 203, 204, 206, 208, 209, 213, 214, 215, 216, 217, 486, 488, 490, 491, 493, 494, 496, 497, 498, and 500;
d. 105, 106, 107, 109, 110, 112, 113, 114, 115, 116, 117, 132, 133, 138, 139, 140, 141, 145, 146, 203, 204, 206, 208, 209, 213, 214, 215, 216, 217, 333, 334, 335, 337, 338, 339, 340, 341, 342, 343, 345, 346, 347, 349, 366, 368, 371, 372, 373, 376, 377, 378, 379, 380, 486, 488, 490, 491, 493, 494, 496, 497, 498, 500, 620, 621, 625, 627, 628, 629, 631, 632, 633, 723, 727, 728, 729, 730, 733, 734, 737, 751, 753, 757, 762, 764, 766, 836, 837, 838, 839, 840, 842, 845, 846, 848, 850, 981, 983, 984, 985, 987, 989, 990, 992, and 993;
e. 105, 106, 107, 109, 110, 113, 114, 115, 116, 117, 120, 132, 133, 139, 140, 141, 143, 145, 146, 203, 204, 206, 209, 212, 213, 214, 215, 216, 217, 333, 334, 335, 337, 338, 339, 340, 341, 342, 343, 344, 345, 346, 349, 366, 368, 369, 370, 371, 372, 376, 378, 379, 380, 486, 489, 491, 492, 493, 496, 497, 498, 499, 500, 621, 625, 627, 628, 629, 630, 631, 632, 633, 725, 728, 729, 730, 731, 733, 735, 737, 751, 756, 760, 762, 763, 764, 837, 838, 839, 840, 842, 844, 845, 846, 847, 850, 983, 984, 986, 987, 989, 990, 992, 993, and 995;
f. 105, 106, 107, 109, 110, 113, 114, 115, 116, 117, 120, 132, 138, 139, 141, 143, 144, 145, 146, 203, 206, 209, 210, 211, 213, 214, 215, 216, 217, 333, 334, 335, 336, 337, 339, 340, 341, 342, 343, 344, 346, 347, 349, 368, 369, 371, 373, 374, 376, 377, 378, 379, 380, 486, 487, 492, 493, 495, 496, 497, 498, 499, 500, 621, 625, 627, 628, 629, 630, 631, 632, 633, 723, 725, 726, 728, 729, 730, 734, 737, 750, 751, 753, 760, 762, 763, 837, 838, 840, 841, 842, 843, 844, 845, 846, 850, 982, 984, 985, 986, 987, 989, 990, 992, and 995; or
g. 12, 14, 21, 25, 46, 59, 60, 91, 95, 109, 126, 163, 224, 225, 241, 242, 282, 293, 303, 319, 320, 343, 347, 349, 350, 380, 400, 407, 415, 440, 457, 460, 461, 464, 467, 469, 478, 479, 480, 507, 508, 530, 559, 560, 608, 617, 642, 655, 663, 671, 686, 725, 736, 737, 741, 777, 784, 793, 798, 820, 824, 834, 856, 870, 885, 890, 898, 902, 903, 911, 965, 975, 986, 993, and 1053.

According to some embodiments, the synonymous mutations comprise at least one group of mutations within SEQ ID NO: 22 selected from:
a. C3T, C144G, C345G, C408T, G534A, T595C, G597T, A663C, C726G, G831C, C894T, C1026A, T1149A, T1209G, C1338T, G1458A, A1521C, C1584T, C1647G, G1710T, A1791G, T1857G, C1920T, T2055C, C2118T, A2370T, T2445A, G2517C, C2578A, G2580A, C2652A, T2856C, G2997A, and A3159T;
b. T36C, C57T, T79C, C102T, G123A, C147T, C168G, C189T, C210A, G258C, C279T, C324G, G348T, G369A, A390C, A432G, T451A, C452G, C474T, A495G, G516A, T537G, C624T, G645C, A666C, C729T, C759T, C780T, T792C, G813A, C834T, C876G, A939G, G960T, A984C, T1008C, C1029G, A1068T, C1089T, C1131T, T1194C, G1212A, T1233C, G1278T, A1299G, G1320A, T1341G, T1365A, C1395T, C1419T, T1459C, G1461T, C1482T, C1503T, A1524T, T1545C, A1564C, G1566C, C1650T, T1669C, G1671T, A1690C, A1692G, C1734T, G1764C, A1794C, C1818G, T1881C, C1900A, T1944C, C1968T, T1989A, C2010T, G2034A, C2079T, G2100A, G2163C, A2203T, G2204C, G2247A, T2268C, C2331T, A2373G, G2397A, C2424G, T2448A, A2469G, C2499T, T2518C, G2520T, A2541G, A2562G, T2583C, T2610G, C2634A, A2655C, C2682T, A2751G, C2772T, A2791C, T2814C, C2838T, A2859T, C2886G, C2931G, C2976T, C3000T, C3051T, T3078C, T3135C, and C3162T;
c. A396G, C397T, G399A, G414T, A417G, T420C, T423A, T435G, T438G, A609C, A612C, G618A, C624T, C627A, T639G, G642A, G645A, C646A, C649A, C651G, G1458A, A1464C, C1470T, T1473G, T1479C, C1482T, G1488C, T1489C, A1494C, and T1500C;
d. C315T, C318T, G321A, C327G, C330T, A334C, G339A, G342C, C345T, G348A, G351A, A396G, C397T, G399A, G414T, A417G, T420C, T423A, T435G, T438G, A609C, A612C, G618A, C624T, C627A, T639G, G642A, G645A, C646A, C649A, C651G, C999T, C1002T, T1005C, T1011C, T1014C, T1017C, C1018T, C1020G, C1023G, C1026T, C1029A, G1035A, C1038A, G1041A, T1047A, A1098G, C1102T, C1104G, G1113T, C1116T, C1119T, T1128C, C1131T, G1134A, C1137G, C1140G, G1458A, A1464C, C1470T, T1473G, T1479C, C1482T, G1488C, T1489C, A1494C, T1500C, G1860A, A1863G, C1875T, T1881G, C1884T, T1887C, C1891T, C1893G, C1896T, C1899T, T2169C, C2181T, A2184G, A2187G, G2190T, C2199T, C2202T, T2211G, A2253G, T2259C, C2271T, T2286C, A2292G, C2296T, G2508C, T2511C, G2514C, G2517C, T2518C, G2526A, C2533A, C2535A, C2536A, C2538G, C2544T, G2550C, A2943G, A2949G, G2952C, C2955T, C2961T, A2967G, T2970C, C2976T, and T2979C;
e. C315T, C318T, G321A, C327G, C330T, G339A, G342T, C345T, G348A, G351A, G360A, A396G, C397T, A417G, T420C, T423G, A429G, T435G, T438G, A609C, A612C, G618C, C627A, G636A, T639G, G642T, G645C, G648T, C649A, C651G, C999A, C1002T, T1005G, T1011C, T1014C, T1017C, C1018T, C1020G, C1023G, C1026T, C1029A, G1032A, G1035A, C1038G, T1047A, A1098G, C1104G, G1107A, A1110G, G1113C, C1116T, T1128C, G1134A, C1137G, C1140T, G1458A, C1467T, T1473C, T1474C, T1479C, G1488C, T1489C, A1494T, C1497T, T1500C, A1863G, C1875T, T1881A, C1884T, T1887A, C1890T, C1893G, C1896A, C1899G, A2173C, A2175G, A2184T, A2187G, G2190C, C2193T, C2199T, A2203T, G2204C, C2205A, T2211G, A2253G, T2268C, A2280T, T2286C, G2289A, A2292G, T2511C, G2514C, G2517C, T2518C, G2526T, C2532A, C2533A, C2535A, C2536A, C2538A, A2539C, A2541G, G2550C, A2949G, G2952T, T2958A, C2961T, C2965A, T2970C, C2976T, T2979C, and G2985A;
f. C315T, C318T, G321A, C327G, C330T, G339A, G342C, C345A, G348A, G351A, G360A, A396C, G414T, A417G, T423G, A429G, A432G, T435A, T438G, A609C, G618A, C627T, G630A, G633A, T639G, G642A, G645T, G648T, C649A, C651G, C999T, C1002T, T1005G, T1008G, T1011G, T1017C, C1018T, C1020G, C1023A, C1026T, C1029A, G1032A, C1038G, G1041A, T1047C, C1102T, C1104G, G1107A, G1113C, C1119T, G1122A, T1128C, C1131T, G1134A, C1137A, C1140A, G1458A, T1459C, T1474C, T1479C, G1485T, G1488C, T1489C, G1491A, A1494T, C1497T, T1500C, A1863G, C1875T, T1881A, C1884T, T1887C, C1890T, C1891T, C1893G, C1896A, C1899T, T2169C, A2173C, A2175G, T2178C, A2184T, A2187G, G2190C, C2202T, T2211G, G2250A, A2253G, T2259C, A2280T, T2286C, G2289A, T2511C, G2514C, T2518C, C2523T, G2526T, C2527A, G2529A, C2532A, C2533A, C2535A, C2536A, C2538A, G2550C, G2946C, G2952T, C2955T, T2958C, C2961T, A2967G, T2970C, C2976T, and G2985A; and
g. T36C, C42G, T63C, T75C, G138C, C177T, C180G, T273C, G285A, C327G, G378T, C489G, A672C, T675C, C723G, C726A, A846G, A879C, C909T, G957C, G960A, C1029A, G1041A, T1047C, C1050A, C1140A, G1200C, C1221A, T1245C, G1320A, T1369A, C1370G, C1380T, T1383C, G1392C, C1401G, A1407T, T1434C, C1437A, T1440C, A1521C, A1524C, T1590C, A1677C, C1680A, T1824C, G1851A, T1926C, T1965C, T1989A, T2011C, G2013C, T2058C, A2173C, A2175G, G2208A, T2211G, T2223C, C2331G, T2352C, G2379A, C2394T, C2460A, T2472C, A2502C, T2568C, T2610A, A2655C, C2670T, T2694C, A2706G, C2709T, T2733C, G2895C, G2925C, T2958C, T2979C, and A3159C.

According to some embodiments, the attenuated HCV variant comprises any one of SEQ ID NO: 24-30.

According to some embodiments, a NS5 gene of the HCV variant comprises any one of SEQ ID NO: 24-30.

According to some embodiments, the NS5 gene
a. comprises fewer than 298 synonymous codon substitutions;
b. does not comprise codon substitutions at all of the following codons in SEQ ID NO: 22: 10, 13, 18, 19, 35, 36, 42, 51, 57, 59, 60, 65, 67, 74, 80, 82, 89, 90, 94, 96, 100, 105, 106, 109, 113, 118, 119, 120, 136, 142, 148, 150, 154, 156, 170, 172, 175, 179, 185, 187, 192, 193, 194, 195, 201, 206, 208, 211, 212, 221, 223, 227, 228, 232, 233, 240, 243, 251, 257, 258, 259, 262, 266, 269, 271, 273, 278, 280, 291, 293, 297, 310, 315, 316, 317, 318, 330, 342, 352, 374, 408, 409, 415, 417, 418, 420, 421, 422, 424, 427, 441, 463, 465, 466, 470, 473, 474, 484, 486, 489, 494, 501, 504, 517, 521, 527, 535, 536, 538, 541, 549, 552, 553, 555, 557, 568, 569, 570, 571, 572, 573, 574, 580, 581, 584, 585, 587, 588, 591, 593, 595, 598, 603, 604, 609, 611, 617, 623, 624, 626, 628, 630, 635, 636, 637, 640, 643, 651, 659, 660, 661, 666, 677, 687, 689, 690, 691, 694, 696, 698, 699, 702, 706, 707, 709, 715, 722, 724, 726, 727, 729, 731, 734, 740, 745, 746, 748, 754, 757, 758, 761, 765, 768, 774, 776, 785, 788, 789, 792, 797, 798, 803, 805, 806, 807, 808, 810, 811, 812, 814, 815, 816, 822, 825, 827, 829, 830, 831, 833, 834, 836, 838, 841, 848, 849, 851, 853, 861, 862, 865, 868, 869, 870, 873, 876, 877, 879, 894, 895, 896, 905, 915, 918, 919, 922, 924, 925, 928, 933, 937, 939, 941, 943, 944, 945, 948, 949, 950, 951, 955, 956, 958, 959, 962, 963, 964, 967, 969, 970, 973, 975, 979, 981, 982, 987, 988, 991, 992, 995, 996, 998, 999, 1002, 1012, 1014, 1017, 1020, 1022, 1023, 1024, 1025, 1028, 1029, 1031, 1033, 1035, 1036, 1041, 1042, 1043, 1044, 1045, 1048, 1050, 1051, 1054, 1055, and 1057;
c. does not comprise SEQ ID NO: 31; or
d. a combination thereof.

According to some embodiments, the attenuated HCV variant is a mutant of a natural isolate, being a synthetic virus, comprising a nucleotide acid selected from: single strand RNA (ssRNA), (dsRNA) double strand RNA, single strand DNA (ssDNA) and double strand DNA (dsDNA) or both; optionally wherein the the nucleotide acid is ssRNA.

According to another aspect, there is provided a vaccine composition comprising the attenuated HCV variant of the invention and a pharmaceutically acceptable carrier, excipient or adjuvant.

According to another aspect, there is provided a method for eliciting a protective immune response against HCV in a subject comprising administering to the subject a prophylactically effective dose of the vaccine of the invention, thereby eliciting a protective immune response in the subject; optionally wherein the eliciting a protective immune response is vaccinating.

According to another aspect, there is provided a method for designing an attenuated Hepatitis C Virus (HCV) variant based on mRNA folding, comprising:
a. obtaining a multiple sequence alignment (MSA) of HCV strains;
b. using a computational algorithm to identify regions within the HCV genome with significant selection for strong or weak RNA folding;
c. introducing a plurality of synonymous mutations in the identified regions to alter the local folding energy (LFE) by at least 15%, thereby disrupting the RNA secondary structures critical for the viral life cycle, while preserving the amino acid sequence of the viral proteins;
d. generating the attenuated HCV variant with the introduced synonymous mutations;
e. evaluating the viral fitness of the attenuated HCV variant by measuring RNA levels, infection percentage, and/or viral spread in liver cells;
f. confirming the genomic stability of the attenuated HCV variant over time without significant reversion to the wild-type sequence;
thereby designing an attenuated HCV variant.

According to another aspect, there is provided a method for designing an attenuated Hepatitis C Virus (HCV) variant based on underrepresented sequences, comprising:
a. obtaining a multiple sequence alignment (MSA) of HCV strains;
b. using a computational algorithm to identify underrepresented (UR) sequences within the HCV genome;
c. introducing a plurality of synonymous mutations to insert the identified UR sequences into their corresponding regions, thereby disrupting the RNA regulatory elements critical for the viral life cycle, while preserving the amino acid sequence of the viral proteins;
d. generating the attenuated HCV variant with the introduced synonymous mutations;
e. evaluating the viral fitness of the attenuated HCV variant by measuring RNA levels, infection percentage, and/or viral spread in liver cells;
f. confirming the genomic stability of the attenuated HCV variant over time without significant reversion to the wild-type sequence;
thereby designing an attenuated HCV variant.

According to some embodiments, at least one of:
a. the multiple sequence alignment (MSA) is obtained from a database comprising at least 100 complete HCV strains;
b. the synonymous mutations are introduced only in codons whose frequency in the MSA column is at least 10%;
c. the evaluation of viral fitness includes measuring the size of viral foci in liver cells;
d. the evaluation of viral fitness includes measuring the percentage of infected liver cells over a period of 4 weeks;
e. the confirmation of genomic stability includes deep sequencing of the NS5A and NS5B genes of the attenuated HCV variant; and
f. the synonymous mutations are introduced in the NS5 gene.

According to some embodiments, at least one of:
a. the computational algorithm used to identify regions with significant selection for strong or weak RNA folding includes a sliding window approach with a window length of 39 nucleotides;
b. the synonymous mutations are designed to change the local folding energy (LFE) by at least 15% in regions with significant selection for strong RNA folding; and
c. the synonymous mutations are designed to change the local folding energy (LFE) by at least 15% in regions with significant selection for weak RNA folding.

According to some embodiments, at least one of:
a. the computational algorithm used to identify underrepresented (UR) sequences includes synonymous codon permutations and synonymous dinucleotide permutations; and
b. the synonymous mutations are designed to insert underrepresented sequences in both frame 1 and frame 2 of the HCV genome.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: A heat-map of the changed codons for variants of the NS5 coding region of ZIKV. The original codons are shown in blue, while the altered codons are shown in red.

FIG. 2: A heat-map of the changed codons for variants of the structural proteins coding region of ZIKV. The original codons are shown in blue, while the altered codons are shown in red.

FIG. 3: A line graph demonstrating the mortality of AG129 mice after vaccination with various synthetic WT and attenuated viruses and prior to challenge with WT ZIKV (**P<0.01, *P<0.05 as compared to placebo treatment).

FIG. 4: A line graph demonstrating average percent weight change of animals during the vaccination period. Vaccine was administered at 28 days prior to infection with the WT virus.

FIG. 5: A dot plot graph demonstrating weight change between days −28 and −10 prior to infection with WT virus in animals vaccinated with various synthetic WT and attenuated viruses (***P<0.001 as compared to synthetic WT ZIKV challenge).

FIG. 6: A line graph demonstrating the survival of mice vaccinated with various synthetic attenuated Zika viruses after challenge with WT Zika virus (***P<0.001, **P<0.01, *P<0.05, as compared with placebo).

FIG. 7: A line graph demonstrating average percent weight change of animals after virus challenge.

FIG. 8: A dot plot graph demonstrating weight change between days 5 and 14 post infection with WT virus in animals vaccinated with various synthetic WT and attenuated viruses (***P<0.001, **P<0.01 as compared to placebo treatment).

FIG. 9: A dot plot graph showing the viral titers from serum collected 3 dpi from animals vaccinated with various synthetic WT and attenuated viruses and challenged with WT ZIKV (**P<0.01, *P<0.05 as compared to placebo treatment).

FIG. 10: A bar graph demonstrating the disease signs observed in mice vaccinated with various synthetic WT and attenuated viruses and challenged with WT ZIKV. Numbers above each bar denoted the number of mice in the group.

FIG. 11: A heat-map of the changed codons for variants of the NS5A/B coding region of HCV. The original codons are shown in blue, while the altered codons are shown in red.

FIG. 12: Table of local folding energy changes generated in the NS5A/B coding region of HCV. The 11 regions correspond to the 11 regions shown in FIG. 11. Regions 1 to 11 proceed from the N-terminus to the C-terminus of the NS5A/B protein.

FIG. 13: An outline of the HCV variant design, experiments, and analysis. Blue rectangles indicate major steps.

FIG. 14: LFE of variants designed as part of approach A compared to WT. Changes in local folding energy (LFE) of variants 3-6 at each nucleotide position. Black graph: LFE of the WT virus. Yellow graph: LFE of the variant virus. Blue graph: difference between the variant LFE and the WT LFE. The LFE values were determined based on a sliding window scheme with window size of 39 nucleotides.

FIG. 15: Changes in the viral synthetic NS5 variants relative to the WT sequence. Heatmap of the changed codons in the variants; the white/blue bars indicate unchanged/changed codons, respectively. The boundary between NSSA and NS5B is marked with a dashed line.

FIGS. 16A-16C: Reduced spread of infection with HCV variants compared to WT. (16A) Immunostaining of Huh7.5 cells infected with HCV variants or WT at 2, 4 and 6 days post infection. Cells were stained with HCV-positive serum and anti-human 488 Alexa fluor as the secondary antibody and visualized by fluorescence microscopy. Green-HCV infection, blue—nuclei (DAPI). (16B) Number of infected cells in each focus. Means are shown ±SD from ten different foci. (16C) Percentage of infected cells observed using immunofluorescence following 1, 2.5 and 4 weeks of infection. Means are shown ±SD from three independent experiments (*p<0.05, **p<0.01, ***p<0.001, ****p<0.0001, Two-way ANOVA).

FIGS. 17A-17C: Variants have reduced RNA levels compared to WT virus. (17A) Quantification of the variants' HCV RNA levels. Huh7.5 cells infected with HCV variants, normalized to non-infected Huh7.5 cells by RT-PCR with primers for the HCV RNA 3′ UTR at 2 days, 4 days and 2 weeks post infection. Relative HCV RNA copies are calculated for Huh7.5 infected cells compared to non-infected Huh7.5 cells per ng of total cellular RNA. Differential expression was calculated using the equation of 2(−ΔΔCt), with the GAPDH as an endogenous control. Mean mRNA levels of HCV are shown ±SD from three independent experiments (*p<0.05, **p<0.01, ***p<0.001, ****p<0.0001, Two-way ANOVA). (17B) Counts of Huh7.5 cells infected with HCV variants and immunostained with anti-core antibody. Cells were sorted by using BD FACSAria™ III. (17C) HCV RNA levels after cell sorting. HCV RNA was extracted and quantified by RT-PCR as in (A). Mean fold changes of mRNA levels (calculated compared to non-infected Huh7.5 cells) are shown ±SD from three independent experiments (*p<0.05, **p<0.01, ***p<0.001, ****p<0.0001, t-test).

FIG. 18: Genetic stability of variant HCVmut7. Blue bars indicate codon positions that were mutated in the original sequence of HCVmut7. Green bars indicate the position and frequency of variants found in HCVmut7 2 weeks post infection. Codon positions are relative to the beginning of NS5A.

FIGS. 19A-19C: Reduced effect on host gene expression in infection with HCV variants compared to WT. (19A) Venn diagram of genes that were differentially expressed in E_{WT/Variant} relative to E_{Control} two weeks post infection. (19B) Upper row: illustration of the 3 E_{Variant}−E_{WT} relationship types: “Restrained” means that the gene was DE between E_{Variant} and E_{WT}, and that the change in E_{Variant} (relative to E_{WT}) is closer to E_{Control}; “Extreme” means that the gene was DE between E_{Variant} and E_{WT}, and that the change in E_{Variant} is farther than E_{Control}; “Similar” means that the gene was not DE between E_{Variant} and E_{WT}. Lower row: The number of genes belonging to “Restrained”, “Extreme” and “Similar” categories, for each variant two weeks post infection. (19C) RNA levels for specific genes in HCV WT/variant-infected Huh7.5-HS cells, as quantified by qRT-PCR with primers specific for the tested genes. Differential expression was calculated using the equation of 2(−ΔΔCt), with the GAPDH as an endogenous control. Mean fold changes of mRNA levels (calculated compared to Huh7.5 control cells) are shown ±SD from three independent experiments (*p<0.05, **p<0.01, ***p<0.001, ****p<0.0001, Two-way ANOVA).

FIGS. 20A-20C: The viral variants cause a weaker perturbation of functional GO terms and biological pathways. (20A) Heatmap of enrichment p-values, 2 weeks post infection, for the terms with the 15 most significant p-values (out of the WT and 3 variants). Upper: top 15 GO:BP terms. Lower: top 15 C2 gene sets related to liver (i.e. that include the word “liver” in their name); the first name indicates the first author of the corresponding paper; “up”/“down” indicates whether the original set consists of upregulated/downregulated genes, respectively. (20B) Heatmap of enrichment p-values, 2 weeks post infection, for all terms that were enriched with at least one WT/variant DEG. Upper: All GO:BP terms. Lower: All C2 gene sets. (20C) Boxplots of the enrichment p-values, 2 weeks post infection, for all terms appearing in (20B). Upper: All GO:BP terms. Lower: All C2 gene sets.

FIGS. 21A-21D: Reduced invasion of cells infected with attenuated virus HCVmut7 compared to WT HCV. (21A) WT-infected and HCVmut7-infected cells were seeded in inserts on Matrigel for 24 hours and then fixed. Filters were stained with crystal violet. (21B) The cells that invaded the lower side on the filters were counted. Means are shown ±SD from three independent experiments (***p<0.001 t-test). (21C) WT-infected and HCVmut7-infected cells were plated on Alexa 488 labelled Gelatine for 72 hours and then fixated. (21D) Degradation was analyzed by quantifying the average degraded area per field using ImageJ software and normalized to the number of cells in each field. n=25 fields per group from three independent experiments. Means are shown ±SD from three independent experiments (*p<0.01 t-test).

### DETAILED DESCRIPTION OF THE INVENTION

The present invention, in some embodiments, provides attenuated forms of virulent viruses, in particular Zika virus (ZIKV) and Hepatitis C virus (HCV). The viruses comprise at least one region of synonymous codon substitution which attenuates the virus while retaining the amino acid sequences of the virulent virus. The present invention further concerns vaccine compositions comprising the attenuated viruses and a method of eliciting a protective immune response in a subject by administering the vaccine compositions.

The invention is based on the surprising finding of various effective combinations of synonymous mutations that produce attenuated forms of ZIKV and HCV. By using synonymous mutations, the replicative fitness of the virus can be lowered, thus attenuating it virulence, while maintaining the amino acid sequences of its proteins. The subject inoculated with the attenuated virus does not get sick but can mount an immune response against the exact proteins that will be present should an infection with the virulent form of the virus occur. In particular, it was surprisingly found that the number of synonymous mutations is not directly correlated with the attenuation, but rather other factors determine overall attenuation. It was discovered that synonymous mutation of both the region encoding the NS5 protein and the structural proteins of the virus produced a superior attenuating effect than just modifying one region. This combined effect was not additive, and the combined effect was exponentially better than mutation of either region alone. Further, although it was found that too many synonymous mutations in one region will kill the virus, rendering it unusable for a vaccine; a comparable number of mutations spread over two regions produced the most robust attenuation and vaccine protection. Once again this surprisingly indicates that it is not the total number of mutations that determines attenuation and efficacy.

By a first aspect, there is provided a modified genome of a virulent virus comprising a region of synonymous codon substitution.

By another aspect, there is provided an attenuated form of a virulent virus comprising a modified genome of the invention.

As used herein, the term “virulent virus” refers to a virus that can infect a host (e.g., a human) and cause disease. The virulent virus (from which the attenuated virus is directly or non-directly derived) may be a wild type or naturally occurring prototype or isolate of variants. However, parent viruses also include mutants specifically created or selected in the laboratory on the basis of real or perceived desirable properties. In some embodiments, the virulent virus is a natural isolate. In some embodiments, the virulent virus is a mutant of a natural isolate. In some embodiments, the virulent virus is a synthetic virus. Accordingly, parent viruses that are candidates for attenuation include mutants of wild type or naturally occurring viruses that have deletions, insertions, amino acid substitutions and the like, and also include mutants which have codon substitutions. Similarly, synthetic forms of natural viruses can also be parent viruses to be attenuated. In one embodiment, a parent virus genome sequence differs from a natural isolate by about 200, 150, 100, 90, 80, 75, 70, 60, 50, 40, 30, 25, 20, 15, 10 or 5 amino acids or fewer. Each possibility represents a separate embodiment of the invention. In another embodiment, the parent sequence differs from a natural isolate by about 30 amino acids or fewer. In another embodiment, the parent sequence differs from a natural isolate by about 20 amino acids or fewer. In yet another embodiment, the parent sequence differs from a natural isolate by about 10 amino acids or fewer.

In some embodiments, the virus is an attenuated virus. In some embodiments, the virus is an attenuated live virus. In some embodiments, the attenuated virus is a vaccine. In some embodiments, the attenuated virus is less virulent than the unattenuated virus. In some embodiments, the attenuated virus replicates more slowly than the unattenuated virus. As used herein, the term “attenuated virus” refers to a virus, in which the virulence thereof has been reduced, e.g., by genetic manipulation of the viral genome.

In some embodiments, the virus is an RNA virus. In some embodiments, the RNA is single stranded RNA. In some embodiments, the virus is Zika virus. In some embodiments, the virus is Hepatitis virus. In some embodiments, the Hepatitis is Hepatitis C. In some embodiments, the synthetic form of an RNA virus comprises a DNA genome. In some embodiments, the attenuated virus comprises a DNA genome. In some embodiments, the attenuated virus comprises a RNA genome. It will be understood that for the purposes of infection and eliciting an immune response the inoculating virus can have a DNA or an RNA genome as the proteins produced which will generate the immune response will be identical. In some embodiments, a synthetic virus comprises a genome comprising a nucleotide acid selected from: single strand RNA (ssRNA), (dsRNA) double strand RNA, single strand DNA (ssDNA) and double strand DNA (dsDNA). In some embodiments, the genome is an ssRNA genome. In some embodiments, the genome is a dsDNA genome.

Generally, modifications (i.e., substitutions) are performed to a point at which the virus can still be grown in some cell lines (including lines specifically engineered to be permissive for a particular virus), but where the virus is avirulent in a normal animal or human. Such avirulent viruses are excellent candidates for either a killed or live vaccine since they encode exactly the same proteins as the fully virulent virus and accordingly provoke exactly the same immune response as the fully virulent virus. In addition, the process described herein offers the prospect for fine tuning the level of attenuation; that is, it provides the capacity to design synthetic viral genomes whose secondary structure is deoptimized to a roughly predictable extent. Design, synthesis, and production of viral particles is achievable in a timeframe of weeks once the genome sequence is known, which has important advantages for the production of vaccines in potential emergencies. Furthermore, the attenuated viruses are expected to have virtually no potential to revert to virulence because of the extremely large numbers of deleterious nucleotide changes involved. This method may be generally applicable to a wide range of viruses, requiring only knowledge of the viral genome sequence and a reverse genetics system for any particular virus.

Methods of modifying viral genomes are known in the art and employ molecular biology techniques such as in vitro transcription, reverse transcription, polymerase chain reaction, restriction digestion, cloning etc.

Detailed descriptions of conventional methods, such as those employed in the construction of recombinant plasmids, transfection of host cells with viral constructs, polymerase chain reaction (PCR), and immunological techniques can be obtained from numerous publications, including Sambrook et al. (1989) and Coligan et al. (1994).

When the viral genome is an RNA genome, they may be isolated from virions or from infected cells, converted to DNA (“cDNA”) by the enzyme reverse transcriptase, possibly modified as desired, and reverted, usually via the RNA intermediate, back into infectious viral particles. Most commonly, the entire cDNA copy of the genome is cloned immediately downstream of a phage T7 RNA polymerase promoter that allows the in vitro synthesis of genome RNA, which is then transfected into cells for generation of virus (van der Wert, et al., 1986). Alternatively, the same DNA plasmid may be transfected into cells expressing the T7 RNA polymerase in the cytoplasm.

In certain embodiments the modifying is achieved by de novo synthesis of DNA containing the synonymous codons and substitution of the corresponding region of the genome with the synthesized DNA. In further embodiments, the entire genome is substituted with the synthesized DNA. In still further embodiments, a portion of the genome is substituted with the synthesized DNA.

In some embodiments, the genome comprises at least one coding sequence. In some embodiments, the coding sequence encodes a protein. In some embodiments, the protein is selected from a functional category of proteins selected from surface proteins, structural proteins, enzymatic proteins, unclassified proteins and proteins of other functions. In some embodiments, the mutation is within the coding sequence. In some embodiments, the coding sequence comprises the at least one mutation. In some embodiments, the region is within a coding sequence.

In some embodiments, a region is a plurality of regions. In some embodiments, a region is two regions. In some embodiments, a region comprises a first region and a second region. In some embodiments, a region comprises sequence that encodes protein. In some embodiments, a region is not within an untranslated region. In some embodiments, a region is a coding region. In some embodiments, the untranslated region of the modified genome is identical to the untranslated region of the genome of the virulent virus. In some embodiments, a region encodes at least one complete viral protein. In some embodiments, a region consists of the sequence that encodes 1, 2 or 3 complete viral proteins. Each possibility represents a separate embodiment of the invention. It will be understood by a skilled artisan that though the region may encode a complete viral protein the synonymous mutations need not be in the very first and very last codon form the complete viral protein, but rather will be distributed throughout the viral protein. Thus, mutations spanning 80%, 85%, 90%, 95% or more of the region coding for a viral protein will be considered to be a region that encodes a complete viral protein. In some embodiments, a viral protein is selected from a structural protein and a non-structural protein. In some embodiments, a structural protein is selected from: protein C, protein E and protein prM. In some embodiments, a structural protein is selected from: core protein, E1 protein, and E2 protein. In some embodiments, a structural protein is selected from: core protein, E1 protein, E2 protein and p7 protein. In some embodiments, a non-structural protein is selected from NS1, NS2A, NS2B, NS3, NS4A, NS4B and NS5. In some embodiments, the non-structural protein is NS5. In some embodiments, a non-structural protein is selected from NS2, NS3, NS4A, NS4B, NS5A and NS5B. In some embodiments, the non-structural protein is NS5A. In some embodiments, the non-structural protein is NS5B. In some embodiments, the non-structural protein is NS5A and NS5B. In some embodiments, at least one structural protein is at least 2, at least 3 or at least 4 structural proteins. Each possibility represents a separate embodiment of the invention. In some embodiments, at least one structural protein is all structural proteins. Again, it will be understood that for a region to include the coding region for all structural proteins it need not include a mutation of the first and last codon, but rather mutations spanning 80%, 85%, 90%, 95% or more of the region coding for all structural proteins will be considered to be a region that encodes all structural proteins.

In some embodiments, a first region encodes at least one non-structural protein, and a second region encodes at least one structural protein or a portion thereof. In some embodiments, a first region encodes an NS5 protein or a portion thereof. In some embodiments, a first region encodes an NS5A or portion thereof and a NS5B protein or portion thereof. In some embodiments, a first region is the region. In some embodiments, a portion comprises at least 20, 25, 30, 35, 40, 45, 50, 55, 60, 65, 70, 75, 80, 85, 90, 92, 95, 97, or 99% of the protein. Each possibility represents a separate embodiment of the invention. In some embodiments, a portion comprises at least 20% of the protein. In some embodiments, a second region encodes a structural protein selected from: protein C or a portion thereof, protein E or a portion thereof and protein prM or a portion thereof. In some embodiments, a second region encodes protein C, protein E and protein prM. In some embodiments, the second region is the region.

In some embodiments, a region comprises or consists of at least 500, 550, 600, 700, 800, 900, 1000, 1500, 2000, 2100, 2200, 2300, 2400, 2500, 2600, 2700, 2800, 2900, 3000 or 3100 nucleotides. Each possibility represents a separate embodiment of the invention. In some embodiments, a region comprises or consists of at least 150 nucleotides. In some embodiments, a region comprises or consists of at least 2000 nucleotides. In some embodiments, a region comprises or consists of at least 2400 nucleotides. In some embodiments, a region comprises or consists of at least 2700 nucleotides. In some embodiments, a region comprises or consists of at least 3000 nucleotides. In some embodiments, a region comprises or consists of at least 3100 nucleotides.

In some embodiments, a region is not a complete genome. In some embodiments, a region consists of coding sequence encoding 1 protein. In some embodiments, a region consists of coding sequence encoding 2 proteins. In some embodiments, a region consists of coding sequence encoding 3 proteins. In some embodiments, a region comprises or consists of at most 2500, 2600, 2700, 2800, 2900, 3000, 3100, 3200, 3300, 3400, 3500, 3600, 3700, 3800, 3900, 4000, 4500, or 5000 nucleotides. Each possibility represents a separate embodiment of the invention. In some embodiments, a region comprises or consists of at most 2500 nucleotides. In some embodiments, a region comprises or consists of at most 2800 nucleotides. In some embodiments, a region comprises or consists of at most 3200 nucleotides.

In some embodiments, a synonymous codon substitution comprises a mutation in a codon that converts the codon to a synonymous one. As used herein, the term “synonymous codon” refers to a codon with a different nucleotide sequence, but which codes for the same amino acid. Synonymous codons are provided in Table 4. “Synonymous” codons are codons that encode the same amino acid. Thus, for example, CUU, CUC, CUA, CUG, UUA, and UUG are synonymous codons that code for Leucine (Leu). Synonymous codons are not used with equal frequency. In general, the most frequently used codons in a particular organism are those for which the cognate tRNA is abundant, and the use of these codons enhances the rate and/or accuracy of protein translation. Conversely, tRNAs for the rarely used codons are found at relatively low levels, and the use of rare codons is thought to reduce translation rate and/or accuracy. Thus, to replace a given codon in a nucleic acid by a synonymous but less frequently used codon is to substitute a “deoptimized” (in terms of speed) codon into the nucleic acid.

In one embodiment, the codons of the RNA are replaced with synonymous codons while maintaining the overall codon bias of the virus. Thus, the overall the average number of rare and/or frequent codons remains the same throughout the RNA. In another embodiment, the codons of the RNA are replaced with synonymous codons thereby altering the overall codon bias of the virus. Thus, the overall average number of rare and/or frequent codons differs from the wild-type virulent virus.

As used herein, a “rare” codon refers to one of at least two synonymous codons encoding a particular amino acid that is present in an mRNA at a significantly lower frequency than the most frequently used codon for that amino acid. Thus, the rare codon may be present for example at about a 2-fold lower frequency than the most frequently used codon. In one embodiment, the rare codon is present at least a 3-fold, more preferably at least a 5-fold, lower frequency than the most frequently used codon for the amino acid. Conversely, a “frequent” codon refers to one of at least two synonymous codons encoding a particular amino acid that is present in an mRNA at a significantly higher frequency than the least frequently used codon for that amino acid. The frequent codon may be present at about a 2-fold, preferably at least a 3-fold, more preferably at least a 5-fold, higher frequency than the least frequently used codon for the amino acid. In some embodiments, codon frequency is within the virus. In some embodiments, codon frequency is within the host infected by the virus. In some embodiments, codon frequency is within a population of natural isolates of the virus. In some embodiments, a rare codon is not a deleterious codon.

In one embodiment, the codons of the RNA are replaced with synonymous codons while maintaining codon pair bias of the virus. In another embodiment, the codons of the RNA are replaced with synonymous codons thereby altering the overall codon pair bias of the virus. Codon pair bias is described in WO 2008121992, the contents of which are incorporated herein by reference.

In some embodiments, a synonymous codon substitution comprises mutation of 1 nucleotide. In some embodiments, the 1 nucleotide is the third nucleotide of the codon. In some embodiments, a synonymous codon substitution comprises mutation of 2 nucleotides. In some embodiments, the 2 nucleotides are the first and third nucleotides of the codon. In some embodiments, a synonymous codon substitution comprises mutation of all 3 nucleotides of the codon.

TABLE 4
Synonymous codons

| | | | |
|---|---|---|---|
| F | UUC/UUU | P | CCC/CCU/CCA/CCG |
| L | CUC/UUG/CUU/CUG/ | T | ACC/ACU/ACA/ACG |
| | CUA/UUA | | |
| I | AUC/AUU/AUA | A | GCC/GCU/GCG/GCA |
| M | AUG | S | UCC/UCU/UCA/UCG/ |
| | | | AGU/AGC |
| V | GUC/GUG/GUU/GUA | Q | CAA/CAG |
| Y | UAC/UAU | N | AAC/AAU |
| STOP | UAA/UAG/UGA | K | AAG/AAA |
| D | GAC/GAU | E | GAG/GAA |
| C | UGU/UGC | W | UGG |
| R | CGU/CGC/CGA/CGG/ | H | CAC/CAU |
| | AGG/AGA | | |
| G | GGU/GGC/GGG/GGA | | |

Introduction of a mutation into a genome is well known in the art. Any known genome editing method may be employed, so long as the mutation is specific to the location and change that is desired. Non-limiting examples of mutation methods include, site-directed mutagenesis, CRISPR/Cas9 and TALEN. In the case of a synthetic genome a genome with the desired sequence can be generated de novo.

In some embodiments, the mutation is a point mutation. In some embodiments, the mutation changes one of the four DNA bases to a different base. In some embodiments, the mutation changes one of the four RNA bases to a different base. It will be understood that in a DNA genome the change will be to another DNA base and in an RNA genome the change will be to another RNA base. In some embodiments, the mutation is within a coding region and is a synonymous mutation. In some embodiments, a synonymous mutation mutates a codon to a synonymous codon. In some embodiments, a synonymous mutation does not alter an amino acid sequence encoded by the coding sequence comprising the mutation. In some embodiments, the mutated coding sequence encodes a protein with an identical amino acid sequence to the protein encoded by an unmutated coding sequence.

In some embodiments, at least one region comprises at least one mutation. In some embodiments, at least one region comprises a plurality of mutations. In some embodiments, at least one region comprises at least 28 mutations. In some embodiments, at least one region comprises at least 29 mutations. In some embodiments, at least one region comprises at least 30 mutations. In some embodiments, at least one region comprises at least 32 mutations. In some embodiments, at least one region comprises at least 60 mutations. In some embodiments, at least one region comprises at least 75 mutations. In some embodiments, at least one region comprises at least 90 mutations. In some embodiments, a plurality of regions comprises at least one mutation. In some embodiments, the first region comprises at least 32 mutations. In some embodiments, the first region comprises at least 29 mutations. In some embodiments, the first region comprises at least 60 mutations. In some embodiments, the first region comprises at least 62 mutations. In some embodiments, the first region comprises at least 90 mutations. In some embodiments, the first region comprises at least 100 mutations. In some embodiments, the first region comprises at least 150 mutations. In some embodiments, the first region comprises at least 160 mutations. In some embodiments, the first region comprises at least 169 mutations. In some embodiments, the second region comprises at least 70 mutations. In some embodiments, the second region comprises at least 75 mutations. In some embodiments, mutations are single nucleotide mutations. In some embodiments, mutations are codon mutations. In some embodiments, mutations are synonymous codon substitutions.

In some embodiments, a region comprises fewer than 170, 180, 190, 200, 210, 220, 230, 240, 250, 260, 270, 280, or 290 mutations. Each possibility represents a separate embodiment of the invention. In some embodiments, a region comprises fewer than 290 mutations. In some embodiments, a region comprises fewer than 200 mutations. In some embodiments, a region comprises fewer mutations that would result in death of the virus. In some embodiments, the synonymous substitutions do not kill the virus.

In some embodiments, the synonymous substitutions reduce or decrease viral protein production rate in an infected cell. In some embodiments, the synonymous substitutions reduce or decrease the replicative fitness of the virus. As used herein the term “replicative fitness” refers to the health of a virus as measured by its capacity to propagate and its speed of virion production. In some embodiments, reduced replicative fitness comprises a lower viral load. In some embodiments, reduced replicative fitness comprises a slower rate of new cell infection. In some embodiments, reduced replicative fitness comprises a decreased free pool of a cellular resource in a cell infected by the virus. In some embodiments, decreased replicative fitness comprises a decreased free ribosome pool in a cell infected by the virus. In some embodiments, decreased replicative fitness comprises a decreased free RNA polymerase (RNAP) pool in a cell infected by the virus. In some embodiments, decreased replicative fitness comprises decreased viral protein production. In some embodiments, decreased replicative fitness comprises a decreased ability to cause symptomatic infection. In some embodiments, decreased replicative fitness comprises decreased virulence. In some embodiments, symptomatic infection is illness. In some embodiments, the replicative fitness is fitness when competing against another virus. In some embodiments, the other virus is the virulent virus.

Methods of decreasing viral fitness, protein production, and virulence by generating synonymous mutations are well known in the art. Any such method may be employed to produce the synonymous mutations of the invention. Such methods include entropy-based methods, rarer codon-based methods, folding deoptimization methods, and underrepresented sequences based methods to name but a few. Such methods are described hereinbelow and are well known in the art. Further, such methods can be found for example in International Patent Publication WO2017056094, WO2018138727 and WO2021205462 and U.S. Pat. No. 11,236,344 hereby incorporated by reference in their entireties.

In some embodiments, the synonymous mutation is produced by an entropy-based approach. In some embodiments, an entropy approach increases entropy. In some embodiments, the synonymous mutation is produced by replacement of a more common codon with a less common codon. In some embodiments, the synonymous substitutions deoptimize the region. In some embodiments, deoptimize is with respect to translation speed. In some embodiments, the synonymous mutations slow translation of the region. In some embodiments, the slowest translating codon is substituted. In some embodiments, the rarest codon is substituted. In some embodiments, a codon is not substituted if it is rare. In some embodiments, a rare codon is a codon that is used less than 10% of the time to encode a given amino acid. In some embodiments, a rare codon is a codon that is used less than 15% of the time to encode a given amino acid. In some embodiments, a deleterious codon is a codon that is used less than 10% of the time to encode a given amino acid. In some embodiments, a deleterious codon is a codon that is used less than 15% of the time to encode a given amino acid. In some embodiments, to encode a given amino acid is to encode a given amino acid at a particular location. In some embodiments, to encode a given amino acid is to encode a given amino acid in a particular virus. In some embodiments, to encode a given amino acid is to encode a given amino acid in a plurality of viruses where all viruses of the plurality are variants of the same virus. In some embodiments, the plurality of viruses is a population of viruses. In some embodiments, a multiple sequence alignment (MSA) is generated for a population of viral genomes and at a location where a synonymous mutation can be made a rare codon is not selected. In some embodiments, rare is a codon that is used less than 10% in the MSA. In some embodiments, rare is a codon that is used less than 15% in the MSA. In some embodiments, rare is a codon that is used less than 10% at a given location in the MSA. In some embodiments, rare is a codon that is used less than 15% at a given location in the MSA. In some embodiments, the less common codon is not a rare codon. In some embodiments, rare is a codon that is used less than 15% at a given location is all variants of a particular virus. In some embodiments, rare is a codon that is used less than 10% at a given location is all variants of a particular virus. In some embodiments, all variants are all isolates.

In some embodiments, the synonymous mutation is produced by a folding based approach. In some embodiments, the folding is deoptimized. In some embodiments, folding is RNA folding. In some embodiments, a region of strong folding is made weaker, or a region of weak folding is made stronger. In some embodiments, the region to have its folding modified is a region of evolutionarily conserved structure. According to this aspect of the present invention, the phrase “evolutionarily conserved structure” refers to a structure or lack thereof, being present in at least 10%, 20%, 30%, 40%, 50%, 60%, 70%, 80%, 90%, 91%, 92%, 93%, 94%, 95%, 96%, 97%, 98%, 99% or 100% of the known serotypes, genotypes, strains, variants or isolates of a particular virus. Each possibility represents a separate embodiment of the invention. Specifically, the % of the strain can be chosen such that the signal will be statistically significant based on an appropriate null model. In one embodiment, the evolutionarily conserved RNA structure refers to a general secondary structure and not to a specific structure per se.

In another embodiment, the evolutionarily conserved RNA structure refers to the presence of a particular structure (e.g., a hairpin structure, a stem and/or a loop). In another embodiment, the evolutionarily conserved RNA structure refers to the absence of a secondary structure. It will be appreciated that when there is a change in structure, there may or may not be a change in folding energy. However, when there is a change in folding energy, this is typically always associated with a change of structure.

In some embodiments, strong folding comprises more negative local folding energy. In some embodiments, weak folding comprises less negative (or positive) local folding energy. In some embodiments, a region of conserved low local folding energy is increased. In some embodiments, a region of conserved higher local folding energy is decreased. In some embodiments, the local folding energy at a region of conserved folding structure is made less negative (increased). In some embodiments, the local folding energy at a region of conserved lack of folding structure is made more negative (decreased). In some embodiments, deoptimizing folding comprises increasing the local folding energy in a region of evolutionarily conserved strong folding, decreasing the local folding energy in a region of evolutionarily conserved weak folding, or both.

The folding energy (FE) is a thermodynamic energy involved in maintaining a secondary structure available to perform physical work while being released, and thus is characterized by non-positive values. mRNA secondary structure is believed to be in the most stable conformation when a minimum amount of free folding energy is exerted (the FE obtains the most negative value). The number and strength of hydrogen bonds in RNA determine the folding energy, which is related to the folding strength of the structure: more negative FE indicates possibly stronger and more stable folding, while less negative FE corresponds to weaker and less structured conformations.

According to one embodiment, a position with weak RNA folding (less negative free energy/higher free energy) is modified to increase the RNA folding thereof (i.e., make the free energy more negative). Positions of weak folding may be defined based on a comparison to a random model that can maintain various basic properties/features of the viral genome (for example, the amino acid content/order, the codon frequencies, the di-nucleotide frequencies, or any combination of these properties/features). If the probability to see weaker folding in this position in the corresponding random genomes is lower than a certain threshold (e.g., 0.05, 0.01, 0.005, 0.001, 0.0001, 0.00001, 0.000001 or the largest p-value that pass correction for multiple hypothesis testing) the position may be defined as a position with weak folding.

According to one embodiment, a position with strong RNA folding (more negative free energy/lower free energy) is modified to decrease the RNA folding thereof (i.e. make the free energy less negative). Positions of strong folding may be defined based on a comparison to a random model that can maintain various basic properties/features of the viral genome (for example, the amino acid content/order, the codon frequencies, the dinucleotide frequencies, or any combination of these properties/features). If the probability to see stronger folding in this position in the corresponding random genomes is lower than a certain threshold (e.g., 0.05, 0.01, 0.005, 0.001, 0.0001, 0.00001, 0.000001 or the largest p-value that pass correction for multiple hypothesis testing) the position may be defined as a position with strong folding.

In some embodiments, the synonymous mutation is produced by an underrepresented sequence approach. In some embodiments, generation of the synonymous codon produces a sequence underrepresented in the viral genome. In some embodiments, the underrepresented sequence is underrepresented in the virus. In some embodiments, the underrepresented sequence is underrepresented in a host of the virus. In some embodiments, the underrepresented sequence is underrepresented in an unmodified genome of the virus. In some embodiments, the underrepresented sequence is underrepresented in the genome of the virus before modification.

In some embodiments, the underrepresented sequence is a sequence of three nucleotides. In some embodiments, the underrepresented sequence is a sequence of four nucleotides. In some embodiments, the underrepresented sequence is a sequence of five nucleotides. In some embodiments, the underrepresented sequence is a sequence of three, four or five nucleotides. It will be understood that the sequence can be in any reading frame and thus can be anywhere within the genome or within a coding sequence. Indeed, a single mutation could generate several underrepresented sequences depending on the nucleotides around it. In some embodiments, the underrepresented sequence is underrepresented in at least 2 reading frames. In some embodiments, the at least 2 reading frames are the first and second reading frames. In some embodiments, the underrepresented sequences are 5-mers. In some embodiments, the underrepresented sequences are selected from those provided in Tables 1 and 3. In some embodiments, the virus is ZIKV and the selected from those provided in Table 1. In some embodiments, the virus is HCV and the selected from those provided in Table 3.

The full Zika virus genome can be found for example at ncbi.nlm.nih.gov/genomes/GenomesGroup.cgi?taxid=64320 as its taxonomy ID is 64320. Further, NCBI provides numerous individual Zika virus isolates. The genome contains approximately 10800 nucleotides. In some embodiments, the Zika virus is isolate SV0010/15. In some embodiments, the Zika virus genome is the genome of isolate SV0010/15. In some embodiments, the Zika virus genome is provided in GenBank accession number KX051562. In some embodiments, the nucleotide sequence encoding the WT Zika NS5 protein is provided in SEQ ID NO: 1. In some embodiments, the region before synonymous substitution comprises SEQ ID NO: 1. In some embodiments, the first region before synonymous substitution comprises SEQ ID NO: 1. In some embodiments, the region before synonymous substitution consists of SEQ ID NO: 1. In some embodiments, the first region before synonymous substitution consists of SEQ ID NO: 1. In some embodiments, SEQ ID NO: 1 provides nucleotides 7668 to 10376 of the Zika genome. In some embodiments, the Zika genome is the genome provided in GenBank accession number KX051562. In some embodiments, the Zika genome is provided in SEQ ID NO: 32. In some embodiments, SEQ ID NO: 1 provides the 3′ end of the Zika genome coding region. In some embodiments, the Zika genome encodes a polyprotein comprising/consisting of the amino acid sequence provided in SEQ ID NO: 33. In some embodiments, SEQ ID NO: 32 encodes SEQ ID NO: 33.

In some embodiments, SEQ ID NO: 1 encodes the C-terminus of the Zika polyprotein. In some embodiments, SEQ ID NO: 1 encodes the Zika NS5 protein. In some embodiments, SEQ ID NO: 1 encodes the amino acid sequence provided in SEQ ID NO: 2. In some embodiments, the region encodes SEQ ID NO: 2. In some embodiments, the first region encodes SEQ ID NO: 2. In some embodiments, the region both before and after synonymous codon substitution encodes SEQ ID NO: 2. In some embodiments, the first region both before and after synonymous codon substitution encodes SEQ ID NO: 2.

In some embodiments, the synonymous substitutions within SEQ ID NO: 1 comprise or consists of substitutions at at least the group of codons consisting of codons number 77, 78, 80, 81, 82, 83, 86, 88, 90, 91, 92, 93, 94, 136, 137, 139, 140, 141, 142, 143, 144, 146, 148, 150, 173, 174, 175, 177, 178, 180, 184, 186, 187, 189, 232, 233, 234, 235, 236, 237, 238, 239, 240, 241, 242, 243, 246, 247, 248, 249, 251, 253, 312, 313, 314, 316, 318, 319, 322, 324, 326, 327, 329, 330, 331, 336, 337, 338, 340, 341, 343, 346, 348, 497, 500, 502, 503, 504, 506, 507, 509, 511, 512, 513, 514, 516, 520, 521, 524, 525, 526, 527, 529, 530, 615, 616, 617, 618, 620, 621, 622, 623, 624, 626, 629, 631, 632, 635, 639, 640, 641, 643, 666, 668, 669, 670, 671, 672, 673, 675, 676, 678, 679, 681, 682, 683, 684, 685, 690, 691, 692, 693, 697, 698, 699, 700, 703, 708, 709, 712, 828, 830, 831, 832, 833, 834, 836, 838, 842, 843, 844, 849, 850, 851, 852, 853, 870, 872, 874, 877, 878, 879, 880, 881, 886, 887, 888, 889, and 890. It will be understood that the codon numbering is with respect to SEQ ID NO: 1, wherein the first 3 nucleotides are codon 1, nucleotides 4-6 are codon 2, nucleotides 7-9 are codon 3 and so on. It will be understood that many amino acids are encoded by more than 2 codons and so there may be multiple possibilities for synonymous substitution of the recited codons.

In some embodiments, the synonymous substitutions within SEQ ID NO: 1 comprise or consist of the group of mutations consisting of: C231G, T234C, T240G, A243G, T246C, C249G, C258A, A262T, G263C, C264A, C270T, C273A, C276T, C279T, C282A, G408C, T411C, G417A, T420C, C423T, G426T, T427C, G429C, G432A, T438C, T444A, T448A, C449G, A450C, A519G, A522G, A525G, A531G, C534A, T540C, T550C, A558C, C561T, A565T, G566C, C567T, A694T, G695C, T696A, G699T, C702A, C705A, G708A, A709T, G710C, C711A, G714A, C717A, C720A, T721C, G723C, G726A, C727A, C729A, G738A, C741T, A742C, G744C, G747A, G753T, T759C, C936A, T939C, A942T, G948C, G954A, T955A, C956G, A966T, G972C, C978T, G981A, G987A, T988A, C989G, A990T, A993G, G1008T, T1011A, A1014G, A1020T, A1023C, T1029C, C1038T, A1044T, C1491T, G1500A, G1506A, C1509T, T1510A, C1511G, A1512C, T1518A, T1521G, A1527G, G1533C, A1536C, T1537C, A1539C, A1542G, C1548G, A1560C, A1563G, A1570T, G1571C, T1572C, C1575G, A1578C, A1581T, A1587C, A1588C, C1845T, C1848T, A1851C, G1854T, G1860A, C1863T, T1866A, C1867A, G1869A, T1872C, G1878A, A1887G, T1891C, G1893A, G1896A, C1905T, C1915T, C1918A, G1923A, G1929A, T1998C, T2004G, G2007T, G2010A, A2013T, T2016A, T2019C, A2023C, G2025C, C2028T, T2034C, C2037T, A2041C, G2043C, C2046T, T2047C, T2052C, T2055C, A2068C, G2073A, C2076T, A2079T, A2091G, C2094T, A2097T, A2100C, C2109T, T2124G, G2127A, T2134A, C2135G, C2484T, C2490T, A2493C, T2496C, G2499C, A2502G, A2508C, T2514C, A2526G, A2529G, A2530C, T2547C, A2550G, T2551A, C2552G, C2556T, A2559C, C2610T, G2616C, A2620C, G2622C, T2631G, T2634C, A2637G, A2640G, A2643G, A2658G, T2659A, C2660G, C2661T, C2664T, A2667G, and G2670T. It will be understood that the numbering of the nucleotides is with respect to SEQ ID NO: 1. Further, the notation of “C231G” means that the cytosine at position 231 in the sequence is mutated to guanine. These mutations are given for a DNA genome of the virus. If the genome is an RNA genome, then the thymines will be replaced with uracils. In some embodiments, the region after synonymous substitution comprises SEQ ID NO: 12. In some embodiments, the region after synonymous substitution consists of SEQ ID NO: 12. In some embodiments, the first region after synonymous substitution comprises SEQ ID NO: 12. In some embodiments, the first region after synonymous substitution consists of SEQ ID NO: 12.

In some embodiments, the nucleotide sequence encoding the WT Zika E protein, C protein and prM protein is provided in SEQ ID NO: 15. In some embodiments, the region before synonymous substitution comprises SEQ ID NO: 15. In some embodiments, the second region before synonymous substitution comprises SEQ ID NO: 15. In some embodiments, the region before synonymous substitution consists of SEQ ID NO: 15. In some embodiments, the second region before synonymous substitution consists of SEQ ID NO: 15. In some embodiments, SEQ ID NO: 15 provides nucleotides 108 to 2519 of the Zika genome. In some embodiments, the Zika genome is the genome provided in GenBank accession number KX051562. In some embodiments, the Zika genome is provided in SEQ ID NO: 32. In some embodiments, SEQ ID NO: 15 provides the 5′ end of the Zika genome coding region.

In some embodiments, SEQ ID NO: 15 encodes the N-terminus of the Zika polyprotein. In some embodiments, SEQ ID NO: 15 encodes the Zika E protein, C protein and prM protein. In some embodiments, SEQ ID NO: 15 encodes the amino acid sequence provided in SEQ ID NO: 16. In some embodiments, the region encodes SEQ ID NO: 16. In some embodiments, the second region encodes SEQ ID NO: 16. In some embodiments, the region both before and after synonymous codon substitution encodes SEQ ID NO: 16. In some embodiments, the second region both before and after synonymous codon substitution encodes SEQ ID NO: 16.

In some embodiments, the synonymous substitutions within SEQ ID NO: 15 comprise or consists of substitutions at at least the group of codons consisting of codons number 5, 24, 42, 47, 53, 57, 64, 71, 88, 92, 104, 107, 112, 138, 147, 154, 163, 164, 189, 194, 203, 216, 221, 222, 224, 233, 247, 258, 264, 265, 276, 299, 304, 319, 329, 330, 339, 344, 377, 378, 385, 394, 403, 410, 422, 431, 433, 442, 456, 468, 476, 482, 497, 503, 511, 516, 532, 538, 541, 550, 554, 558, 583, 595, 613, 614, 622, 628, 640, 648, 653, 654, 662, 667, and 674. In some embodiments, the synonymous substitutions within SEQ ID NO: 15 comprise or consists of substitutions at at least the group of codons consisting of codons number 28, 33, 49, 67, 118, 128, 137, 141, 146, 152, 155, 157, 172, 174, 181, 205, 227, 235, 241, 244, 255, 259, 284, 294, 303, 313, 321, 323, 338, 341, 351, 362, 386, 393, 411, 414, 416, 424, 425, 439, 451, 453, 461, 470, 472, 479, 491, 494, 498, 505, 521, 533, 544, 548, 563, 569, 578, 600, 608, 633, 661, 668, 681, 688, 694, 699, 702, 714, 721, 728, 731, 735, 736, 762, and 781. In some embodiments, the synonymous substitutions within SEQ ID NO: 15 comprise or consists of a combination of the above recited codons such that at least 75 codons are substituted. It will be understood that the codon numbering is with respect to SEQ ID NO: 15, wherein the first 3 nucleotides are codon 1, nucleotides 4-6 are codon 2, nucleotides 7-9 are codon 3 and so on. It will be understood that many amino acids are encoded by more than 2 codons and so there may be multiple possibilities for synonymous substitution of the recited codons.

In some embodiments, the synonymous substitutions within SEQ ID NO: 15 comprise or consist of the group of mutations consisting of: A15G, G72A, G126A, C141T, T159C, G171A, T192C, A213C, G264T, C274T, A312G, T321C, T336C, A414G, T441C, T462C, T489C, A492G, T567C, G582A, C609T, T648C, C663T, T666C, T672A, C699T, C741T, G774T, C792T, T795C, C828T, G897A, T912C, T957C, G987A, T990A, A1017G, G1032C, C1131T, T1134C, G1155A, A1182T, C1207T, A1230T, A1266G, C1291T, T1299C, C1326T, G1368A, C1404A, C1426T, A1428G, G1446A, T1491C, T1509G, T1533C, C1548T, C1594T, A1614C, A1623G, T1650C, A1662C, C1674T, G1749A, C1785T, C1839T, G1842A, C1866T, T1884C, A1920G, T1942C, T1959G, A1962G, T1986A, A2001G, and C2022T. In some embodiments, the synonymous substitutions within SEQ ID NO: 15 comprise or consist of the group of mutations consisting of: G84A, C97T, G147A, T201C, C354T, T384C, C411T, T423C, T438C, G456A, G465A, T471C, C516T, A522G, G543A, C615T, C679T, T703C, G705A, A723G, T730C, T765C, T775C, G777C, C850T, A882T, A909G, T939C, C963T, A969G, A1014G, C1023T, T1053C, C1086T, A1158G, T1179C, C1233T, A1242G, C1248T, T1272C, C1273T, T1317C, T1353C, T1359C, C1383T, C1408T, T1416C, T1437C, T1471C, T1480C, C1494T, G1515A, C1563T, A1599G, T1632C, A1644G, G1689A, T1707A, C1734T, A1800G, G1824A, T1899C, C1983T, T2004C, C2043T, C2064T, C2082T, A2097G, A2106G, T2140C, T2163C, C2184T, T2191C, C2205T, T2208C, G2286A, T2341C, and A2343G. In some embodiments, the synonymous substitutions within SEQ ID NO: 15 comprise or consists of a combination of the above recited nucleotide mutations such that at least 75 codons are substituted. It will be understood that the numbering of the nucleotides is with respect to SEQ ID NO: 15. Further, the notation of “A15G” means that the adenine at position 15 in the sequence is mutated to guanine. These mutations are given for a DNA genome of the virus. If the genome is an RNA genome, then the thymines will be replaced with uracils. In some embodiments, the region after synonymous substitution comprises SEQ ID NO: 17. In some embodiments, the second region after synonymous substitution comprises SEQ ID NO: 17. In some embodiments, the region after synonymous substitution consists of SEQ ID NO: 17. In some embodiments, the second region after synonymous substitution consists of SEQ ID NO: 17. In some embodiments, the region after synonymous substitution comprises SEQ ID NO: 18. In some embodiments, the second region after synonymous substitution comprises SEQ ID NO: 18. In some embodiments, the region after synonymous substitution consists of SEQ ID NO: 18. In some embodiments, the second region after synonymous substitution consists of SEQ ID NO: 18.

In some embodiments, the synonymous substitutions within SEQ ID NO: 1 do not comprise or consists of substitutions at at least the group of codons consisting of codons number 5, 6, 8, 10, 23, 25, 29, 30, 32, 41, 43, 44, 45, 48, 51, 56, 58, 60, 61, 63, 69, 71, 73, 76, 81, 83, 84, 92, 93, 94, 102, 103, 104, 106, 116, 118, 127, 128, 130, 131, 132, 141, 147, 149, 150, 151, 153, 154, 156, 162, 163, 168, 172, 176, 178, 181, 183, 186, 187, 188, 189, 196, 198, 200, 201, 203, 204, 205, 207, 208, 209, 210, 213, 216, 218, 223, 228, 230, 232, 233, 234, 235, 239, 243, 248, 249, 250, 251, 255, 257, 259, 260, 265, 266, 268, 272, 274, 281, 285, 286, 298, 300, 302, 303, 306, 309, 312, 317, 324, 325, 328, 330, 335, 336, 337, 338, 340, 341, 342, 346, 353, 354, 356, 360, 361, 363, 364, 365, 385, 392, 396, 399, 403, 406, 408, 409, 412, 413, 416, 421, 426, 427, 430, 435, 436, 440, 443, 445, 446, 447, 452, 453, 454, 459, 461, 463, 472, 483, 490, 492, 495, 501, 504, 505, 512, 517, 528, 529, 533, 537, 538, 541, 542, 549, 556, 561, 562, 572, 573, 574, 578, 580, 583, 584, 585, 590, 593, 597, 598, 599, 602, 604, 605, 606, 608, 610, 613, 615, 616, 618, 619, 623, 627, 628, 632, 643, 646, 653, 657, 663, 668, 671, 675, 676, 677, 679, 680, 681, 683, 689, 692, 693, 698, 700, 701, 715, 716, 717, 720, 721, 723, 725, 727, 731, 732, 733, 745, 747, 749, 750, 751, 752, 753, 757, 759, 767, 769, 771, 773, 776, 780, 785, 786, 788, 791, 792, 793, 794, 796, 798, 799, 801, 802, 803, 810, 814, 817, 818, 821, 822, 823, 828, 829, 836, 837, 839, 844, 846, 850, 853, 854, 856, 858, 862, 864, 868, 869, 875, 876, 886, 887, 891, 892, 897, 899, and 901. In some embodiments, the first region will be devoid of a combination of synonymous substitutions at the above recited codons. In some embodiments, the region will be devoid of a combination of synonymous substitutions at the above recited codons. It will be understood that the codon numbering is with respect to SEQ ID NO: 1, wherein the first 3 nucleotides are codon 1, nucleotides 4-6 are codon 2, nucleotides 7-9 are codon 3 and so on. It will be understood that many amino acids are encoded by more than 2 codons and so there may be multiple possibilities for synonymous substitution of the recited codons.

In some embodiments, the region does not comprise SEQ ID NO: 14. In some embodiments, the first region does not comprise SEQ ID NO: 14. In some embodiments, the region is devoid of SEQ ID NO: 14. In some embodiments, the first region is devoid of SEQ ID NO: 14. In some embodiments, the modified genome is devoid of SEQ ID NO: 14.

In some embodiments, the synonymous substitutions within SEQ ID NO: 1 comprise or consists of substitutions at at least the group of codons consisting of codons number 1, 17, 59, 79, 129, 158, 179, 227, 258, 276, 290, 310, 322, 349, 374, 422, 448, 469, 487, 516, 536, 559, 576, 595, 636, 655, 694, 718, 743, 809, 833, and 865.

In some embodiments, the synonymous substitutions within SEQ ID NO: 1 comprise or consist of the group of mutations consisting of: G3A, C51T, T177C, T237C, G387A, A474G, T537C, C681T, T774C, G828A, G870A, T930C, A966G, G1047A, T1122G, T1266A, C1344T, C1407A, C1461T, C1548G, T1608C, G1677A, C1728T, T1785C, T1906C, T1965C, A2082G, C2154G, T2229A, A2427G, G2499A, and T2595C. In some embodiments, the region after synonymous substitution comprises SEQ ID NO: 3. In some embodiments, the region after synonymous substitution consists of SEQ ID NO: 3.

In some embodiments, the synonymous substitutions within SEQ ID NO: 1 comprise or consist of the group of mutations consisting of: G3T, C51T, A175T, G176C, T177G, T237C, G387T, A474G, T537C, A679T, G680C, C681G, T774C, G828A, G870A, T930C, A966G, G1047A, T1122A, T1266G, C1344T, C1407G, C1461T, C1548A, T1608G, G1677A, C1728T, T1785C, T1906C, G1908A, T1965C, A2082G, C2154A, T2229G, A2427G, G2499A, and T2595C. In some embodiments, the region after synonymous substitution comprises SEQ ID NO: 11. In some embodiments, the region after synonymous substitution consists of SEQ ID NO: 11.

In some embodiments, the synonymous substitutions within SEQ ID NO: 1 comprise or consists of substitutions at at least the group of codons consisting of codons number 3, 11, 33, 38, 46, 50, 62, 70, 77, 80, 90, 98, 105, 124, 133, 143, 148, 159, 164, 170, 180, 190, 206, 215, 225, 241, 246, 261, 271, 278, 288, 292, 296, 305, 311, 316, 320, 326, 332, 350, 355, 366, 384, 389, 395, 404, 411, 423, 434, 439, 464, 467, 470, 480, 484, 496, 502, 511, 518, 524, 530, 540, 553, 560, 565, 570, 579, 591, 603, 611, 617, 622, 630, 638, 649, 656, 670, 687, 695, 704, 710, 719, 728, 740, 746, 756, 778, 807, 812, 825, 831, 841, 851, 860, 867, 885, and 898.

In some embodiments, the synonymous substitutions within SEQ ID NO: 1 comprise or consist of the group of mutations consisting of: A9G, A33G, C99T, A114G, C138T, G150A, G186C, C210T, C231T, T240C, C270T, A294G, A315G, C372T, T399C, T427C, T444C, A477T, C492G, T510C, T540C, T570C, C616T, T645A, G675A, T721C, G738C, T783A, A813G, T834C, G864A, G876A, C888T, T915C, G933A, G948A, T960C, C978T, C996T, T1050C, T1065C, C1098T, A1152G, C1167T, T1185C, T1212G, G1233A, A1269T, T1302C, A1317G, G1392A, A1401G, G1410A, C1438T, A1440G, T1452C, T1488C, G1506A, C1531T, T1554C, T1572C, G1590A, C1620T, A1659G, A1680G, T1693C, C1710T, A1737G, T1773G, C1809T, T1833C, C1849T, A1851G, T1866C, T1890G, C1912T, T1945C, G1968A, G2010A, A2061G, G2085A, C2112T, T2130C, T2157C, T2184C, C2220T, G2238A, A2268G, C2334T, C2421T, T2436C, C2475T, A2493T, T2521C, T2553C, C2580T, C2601T, C2655T, and T2694C. In some embodiments, the region after synonymous substitution comprises SEQ ID NO: 4. In some embodiments, the region after synonymous substitution consists of SEQ ID NO: 4.

In some embodiments, the synonymous substitutions within SEQ ID NO: 1 comprise or consists of substitutions at at least the group of codons consisting of codons number 136, 137, 139, 142, 143, 146, 147, 148, 149, 150, 312, 313, 314, 316, 317, 318, 319, 320, 321, 322, 323, 324, 325, 326, 327, 328, 329, 330, and 332.

In some embodiments, the synonymous substitutions within SEQ ID NO: 1 comprise or consist of the group of mutations consisting of: G408C, T411C, G417T, G426T, T427C, G429A, T438C, A441T, T444C, G447A, A450C, C936T, T939A, A942T, G948A, T949A, C950G, A951C, G954T, C957A, T960A, G963T, A966T, C969T, G972C, T975G, C978G, A979C, G981C, C982T, C984G, G987T, A990C, and C996A. In some embodiments, the region after synonymous substitution comprises SEQ ID NO: 5. In some embodiments, the region after synonymous substitution consists of SEQ ID NO: 5.

In some embodiments, the synonymous substitutions within SEQ ID NO: 1 comprise or consists of substitutions at at least the group of codons consisting of codons number 77, 79, 80, 81, 83, 84, 85, 86, 88, 89, 92, 93, 94, 136, 137, 139, 142, 143, 146, 147, 148, 149, 150, 312, 313, 314, 316, 317, 318, 319, 320, 321, 322, 323, 324, 325, 326, 327, 328, 329, 330, 332, 337, 338, 339, 342, 343, 345, 615, 617, 618, 619, 621, 622, 623, 624, 626, 627, 628, 631, 632, 636, 638, 639, 642, 643, 644, 645, 669, 670, 671, 672, 673, 675, 678, 680, 681, 683, 684, 687, 689, 690, 692, 695, 699, 701, 703, 706, 708, 709, 710, 712, 713, 714, 870, 872, 873, 874, 877, 878, 881, 884, and 886.

In some embodiments, the synonymous substitutions within SEQ ID NO: 1 comprise or consist of the group of mutations consisting of: C231G, T237C, C238T, T240G, A243C, C249G, A250C, A252G, G255A, C258G, A262T, G263C, C267T, C276T, C279A, C282T, G408C, T411C, G417T, G426T, T427C, G429A, T438C, A441T, T444C, G447A, A450C, C936T, T939A, A942T, G948A, T949A, C950G, A951C, G954T, C957A, T960A, G963T, A966T, C969T, G972C, T975G, C978G, A979C, G981C, C982T, C984G, G987T, A990C, C996A, T1011A, A1014C, C1017T, A1026T, T1029A, C1035T, C1845A, A1851C, G1854C, G1857C, C1863G, T1866A, C1867A, T1872C, G1878A, T1881C, G1884A, T1891C, G1896A, T1906C, G1914C, G1917A, A1926T, G1929A, A1932G, G1935C, G2007C, G2010A, A2013C, T2016C, T2019C, A2023C, T2034C, C2038T, C2040G, A2041C, G2043C, T2047C, G2049T, T2052C, A2061C, T2067C, A2068C, C2076T, G2085A, T2095A, C2096G, A2097T, A2103C, C2109T, A2118G, T2124G, G2127T, T2130C, C2136A, C2139T, T2142C, C2610T, G2616T, C2619G, A2620C, G2622C, T2631A, T2634C, A2643G, C2652T, and A2658G. In some embodiments, the region after synonymous substitution comprises SEQ ID NO: 6. In some embodiments, the region after synonymous substitution consists of SEQ ID NO: 6.

In some embodiments, the synonymous substitutions within SEQ ID NO: 1 comprise or consists of substitutions at at least the group of codons consisting of codons number 77, 78, 80, 83, 85, 86, 88, 89, 90, 91, 92, 93, 94, 136, 137, 139, 142, 143, 144, 146, 148, 150, 312, 313, 314, 316, 317, 318, 319, 321, 322, 324, 327, 328, 329, 330, 332, 337, 338, 340, 341, 342, 343, 615, 618, 619, 620, 622, 623, 624, 626, 627, 628, 631, 632, 636, 638, 639, 640, 641, 642, 643, 645, 667, 669, 670, 671, 672, 674, 675, 676, 678, 679, 680, 681, 682, 685, 690, 691, 695, 698, 699, 701, 703, 704, 708, 709, 711, 712, 713, 714, 872, 873, 874, 875, 876, 877, 878, 879, 880, 881, 884, 885, 886, and 887.

In some embodiments, the synonymous substitutions within SEQ ID NO: 1 comprise or consist of the group of mutations consisting of: C231T, T234A, C238T, T240G, C249A, G255A, C258A, C264T, C267T, C270T, C273A, C276A, C279A, C282A, G408T, T411A, G417A, G426T, T427C, G429C, G432C, T438C, T444G, T448A, C449G, A450T, C936A, T939A, A942T, G948A, A951C, G954A, C957A, G963C, A966T, G972C, G981A, C984A, G987A, T988A, C989G, A990T, C996A, T1011A, A1014G, A1020T, A1023C, A1026T, T1029C, C1845A, G1854T, G1857T, G1860A, T1866A, C1867A, G1869A, T1872C, G1878A, T1881A, G1884A, T1891C, G1893A, G1896A, T1906C, G1908A, G1914A, G1917C, C1918A, G1920A, G1923A, A1926C, G1929A, G1935T, C2001T, G2007T, G2010A, A2013T, T2016A, T2022C, A2023C, G2025C, C2028T, T2034C, C2037T, C2038T, C2040G, G2043A, C2046T, T2055C, A2068C, G2070C, G2073A, G2085A, C2094A, T2095A, C2096G, A2097T, A2103C, C2109T, C2112T, T2124G, G2127A, C2133T, C2136A, C2139T, T2142C, G2616T, C2617A, C2619A, A2620C, C2625A, A2628C, T2631C, T2634C, A2637G, A2640G, A2643G, C2652T, C2655T, A2658G, and C2661T. In some embodiments, the region after synonymous substitution comprises SEQ ID NO: 8. In some embodiments, the region after synonymous substitution consists of SEQ ID NO: 8.

In some embodiments, the synonymous substitutions within SEQ ID NO: 1 comprise or consists of substitutions at at least the group of codons consisting of codons number 1, 17, 59, 77, 78, 79, 80, 81, 82, 83, 86, 88, 90, 91, 92, 93, 94, 129, 136, 137, 139, 140, 141, 142, 143, 144, 146, 148, 150, 158, 179, 227, 258, 276, 290, 310, 312, 313, 314, 316, 318, 319, 322, 324, 326, 327, 329, 330, 331, 336, 337, 338, 340, 341, 343, 346, 348, 349, 374, 422, 44, 469, 487, 516, 536, 559, 576, 595, 615, 616, 617, 618, 620, 621, 622, 623, 624, 626, 629, 631, 632, 635, 636, 639, 640, 641, 643, 655, 666, 668, 669, 670, 671, 672, 673, 675, 676, 678, 679, 681, 682, 683, 684, 685, 690, 691, 692, 693, 694, 697, 698, 699, 700, 703, 708, 709, 712, 718, 743, 809, 833, 865, 870, 872, 874, 877, 878, 879, 880, 881, 886, 887, 888, 889, and 890.

In some embodiments, the synonymous substitutions within SEQ ID NO: 1 comprise or consist of the group of mutations consisting of: G3A, C51T, T177C, C231G, T234C, T237C, T240G, A243G, T246C, C249G, C258A, A262T, G263C, C264A, C270T, C273A, C276T, C279T, C282A, G387A, G408C, T411C, G417A, T420C, C423T, G426T, T427C, G429C, G432A, T438C, T444A, T448A, C449G, A450C, A474G, T537C, C681T, T774C, G828A, G870A, T930C, C936A, T939C, A942T, G948C, G954A, T955A, C956G, A966G, G972C, C978T, G981A, G987A, T988A, C989G, A990T, A993G, G1008T, T1011A, A1014G, A1020T, A1023C, T1029C, C1038T, A1044T, G1047A, T1122G, T1266A, C1344T, C1407A, C1461T, C1548G, T1608C, G1677A, C1728T, T1785C, C1845T, C1848T, A1851C, G1854T, G1860A, C1863T, T1866A, C1867A, G1869A, T1872C, G1878A, A1887G, T1891C, G1893A, G1896A, C1905T, T1906C, C1915T, C1918A, G1923A, G1929A, T1965C, T1998C, T2004G, G2007T, G2010A, A2013T, T2016A, T2019C, A2023C, G2025C, C2028T, T2034C, C2037T, A2041C, G2043C, C2046T, T2047C, T2052C, T2055C, A2068C, G2073A, C2076T, A2079T, A2082G, A2091G, C2094T, A2097T, A2100C, C2109T, T2124G, G2127A, T2134A, C2135G, C2154G, T2229A, A2427G, G2499A, T2595C, C2610T, G2616C, A2620C, G2622C, T2631G, T2634C, A2637G, A2640G, A2643G, A2658G, T2659A, C2660G, C2661T, C2664T, A2667G, and G2670T. In some embodiments, the region after synonymous substitution comprises SEQ ID NO: 9. In some embodiments, the region after synonymous substitution consists of SEQ ID NO: 9.

In some embodiments, the synonymous substitutions within SEQ ID NO: 1 comprise or consists of substitutions at at least the group of codons consisting of codons number 1, 7, 17, 49, 59, 68, 79, 89, 108, 129, 142, 158, 161, 179, 184, 227, 237, 258, 263, 276, 290, 310, 314, 322, 331, 349, 352, 374, 382, 402, 422, 432, 448, 462, 469, 477, 487, 500, 516, 522, 536, 544, 559, 564, 576, 582, 595, 601, 636, 640, 655, 666, 694, 718, 724, 743, 755, 809, 816, 833, 847, and 865.

In some embodiments, the synonymous substitutions within SEQ ID NO: 1 comprise or consist of the group of mutations consisting of: G3A, C21T, C51T, A147C, T177C, C202A, G204A, T237C, C267T, T324C, G387A, G426T, A474G, G483A, T537C, T550C, C681T, C711T, T774C, G789A, G828A, G870A, T930C, A942C, A966G, A993G, G1047A, G1056A, T1122G, T1144C, C1206T, T1266A, C1296T, C1344T, A1386G, C1407A, T1431C, C1461T, G1500A, C1548G, G1566A, T1608C, C1632T, G1677A, C1692T, C1728T, T1746C, T1785C, G1803A, T1906C, C1918A, T1965C, T1998C, A2082G, C2154G, G2172A, T2229A, C2265T, A2427G, C2448T, G2499A, T2539C, and T2595C. In some embodiments, the region after synonymous substitution comprises SEQ ID NO: 10. In some embodiments, the region after synonymous substitution consists of SEQ ID NO: 10.

In some embodiments, the synonymous substitutions within SEQ ID NO: 1 comprise or consists of substitutions at at least the group of codons consisting of codons number 3, 4, 7, 8, 15, 16, 22, 43, 44, 47, 57, 65, 68, 84, 116, 125, 126, 142, 143, 161, 163, 164, 168, 172, 195, 197, 198, 205, 208, 211, 222, 262, 263, 267, 303, 320, 325, 336, 338, 339, 354, 355, 370, 378, 382, 393, 394, 410, 413, 434, 435, 445, 480, 485, 489, 507, 508, 511, 512, 513, 515, 520, 548, 552, 553, 564, 565, 567, 579, 593, 606, 607, 610, 617, 618, 631, 638, 649, 668, 669, 679, 680, 741, 742, 775, 776, 790, 794, 810, 812, 813, 817, 836, 837, 851, 858, 859, 872, and 901.

In some embodiments, the synonymous substitutions within SEQ ID NO: 1 comprise or consist of the group of mutations consisting of: A9G, A12T, C21A, G24T, C45G, G48C, G66C, C129A, C132T, T141G, C169A, A171G, T193C, G195C, C202A, A252G, G348T, T375G, T378C, G426A, T427C, G429T, G483T, A489G, C492G, G504T, T516C, G585C, A591G, G594C, A615G, A624G, C633T, C666G, C786G, G789T, A801T, A909T, T958A, C959G, T960C, T975G, G1008T, A1014G, C1017G, A1062G, T1065G, C1110G, C1134T, T1144C, G1146C, A1179G, C1182G, T1228C, A1230T, A1239C, T1302A, A1305T, A1335G, A1440C, A1455C, C1467A, T1521G, T1524G, G1533C, A1536G, T1537C, A1545G, A1560C, G1644C, T1656A, A1659T, C1692A, T1693C, G1695T, T1699C, G1701T, A1737T, C1779T, T1818G, C1821T, T1830A, A1851G, G1854T, T1891C, G1893C, G1914C, T1945C, G1947C, T2004G, G2007T, C2037A, C2040T, C2223G, C2226G, A2325G, G2328C, T2370G, A2382G, C2430T, T2436G, G2439T, A2451G, A2508T, C2511T, T2551A, C2552G, T2553C, C2574G, C2577T, G2616T, and A2703G. In some embodiments, the region after synonymous substitution comprises SEQ ID NO: 13. In some embodiments, the region after synonymous substitution consists of SEQ ID NO: 13.

In some embodiments, the synonymous substitutions within SEQ ID NO: 15 comprise or consists of substitutions at at least the group of codons consisting of codons number 5, 24, 42, 47, 53, 57, 64, 71, 88, 92, 104, 107, 112, 138, 147, 154, 163, 164, 189, 194, 203, 216, 221, 222, 224, 233, 247, 258, 264, 265, 276, 299, 304, 319, 329, 330, 339, 344, 377, 378, 385, 394, 403, 410, 422, 431, 433, 442, 456, 468, 476, 482, 497, 503, 511, 516, 532, 538, 541, 550, 554, 558, 583, 595, 613, 614, 622, 628, 640, 648, 653, 654, 662, 667, and 674.

In some embodiments, the synonymous substitutions within SEQ ID NO: 15 comprise or consist of the group of mutations consisting of: A15G, G72A, G126A, C141T, T159C, G171A, T192C, A213C, G264T, C274T, A312G, T321C, T336C, A414G, T441C, T462C, T489C, A492G, T567C, G582A, C609T, T648C, C663T, T666C, T672A, C699T, C741T, G774T, C792T, T795C, C828T, G897A, T912C, T957C, G987A, T990A, A1017G, G1032C, C1131T, T1134C, G1155A, A1182T, C1207T, A1230T, A1266G, C1291T, T1299C, C1326T, G1368A, C1404A, C1426T, A1428G, G1446A, T1491C, T1509G, T1533C, C1548T, C1594T, A1614C, A1623G, T1650C, A1662C, C1674T, G1749A, C1785T, C1839T, G1842A, C1866T, T1884C, A1920G, T1942C, T1959G, A1962G, T1986A, A2001G, and C2022T. In some embodiments, the region after synonymous substitution comprises SEQ ID NO: 17. In some embodiments, the region after synonymous substitution consists of SEQ ID NO: 17.

In some embodiments, the synonymous substitutions within SEQ ID NO: 15 comprise or consists of substitutions at at least the group of codons consisting of codons number 28, 33, 49, 67, 118, 128, 137, 141, 146, 152, 155, 157, 172, 174, 181, 205, 227, 235, 241, 244, 255, 259, 284, 294, 303, 313, 321, 323, 338, 341, 351, 362, 386, 393, 411, 414, 416, 424, 425, 439, 451, 453, 461, 470, 472, 479, 491, 494, 498, 505, 521, 533, 544, 548, 563, 569, 578, 600, 608, 633, 661, 668, 681, 688, 694, 699, 702, 714, 721, 728, 731, 735, 736, 762, and 781.

In some embodiments, the synonymous substitutions within SEQ ID NO: 15 comprise or consist of the group of mutations consisting of: G84A, C97T, G147A, T201C, C354T, T384C, C411T, T423C, T438C, G456A, G465A, T471C, C516T, A522G, G543A, C615T, C679T, T703C, G705A, A723G, T730C, T765C, T775C, G777C, C850T, A882T, A909G, T939C, C963T, A969G, A1014G, C1023T, T1053C, C1086T, A1158G, T1179C, C1233T, A1242G, C1248T, T1272C, C1273T, T1317C, T1353C, T1359C, C1383T, C1408T, T1416C, T1437C, T1471C, T1480C, C1494T, G1515A, C1563T, A1599G, T1632C, A1644G, G1689A, T1707A, C1734T, A1800G, G1824A, T1899C, C1983T, T2004C, C2043T, C2064T, C2082T, A2097G, A2106G, T2140C, T2163C, C2184T, T2191C, C2205T, T2208C, G2286A, T2341C, and A2343G. In some embodiments, the region after synonymous substitution comprises SEQ ID NO: 18. In some embodiments, the region after synonymous substitution consists of SEQ ID NO: 18.

In some embodiments, the synonymous substitutions within SEQ ID NO: 15 comprise or consists of substitutions at at least the group of codons consisting of codons number 108, 110, 112, 113, 114, 116, 118, 119, 120, 123, 252, 253, 255, 256, 257, 258, 260, 262, 263, 265, 268, 269, 270, 370, 371, 373, 376, 378, 381, 541, 542, 543, 545, 546, 547, 548, 549, 550, 551, 555, 557, 558, 560, 704, 706, 707, 708, 712, 713, 714, 715, 716, 717, 718, 739, 740, 741, 742, 743, 746, 747, 749, 751, 753, 759, 760, 761, 766, 767, 768, 769, 770, 771, 772, and 773.

In some embodiments, the synonymous substitutions within SEQ ID NO: 15 comprise or consist of the group of mutations consisting of: T324C, C330G, T336C, T339C, C342G, A348C, C354A, A357C, C360A, G369C, C756T, G759A, T765A, C768G, C771T, G774T, A780C, T786C, C789T, T795A, T802C, G804A, A807C, A808T, G809C, C810A, C1110T, C1113T, C1119T, T1126A, C1127G, A1128T, T1134A, C1143G, A1623G, A1624C, A1629G, C1635G, A1638C, T1641G, A1644C, G1647C, A1648T, G1649C, A1653G, T1665G, G1671C, C1674A, T1680A, T2112A, A2116C, T2121A, C2124T, A2136C, C2139G, T2140C, G2142T, A2145G, C2148T, A2151T, C2154T, T2217C, A2220C, A2223C, C2226T, C2229T, T2236C, G2238T, T2241C, A2247G, C2253T, C2259T, A2277G, G2280C, T2281C, G2283C, T2298G, C2299T, T2304C, A2307T, G2310A, T2313C, A2316G, and T2319C. In some embodiments, the region after synonymous substitution comprises SEQ ID NO: 19. In some embodiments, the region after synonymous substitution consists of SEQ ID NO: 19.

In some embodiments, the synonymous substitutions within SEQ ID NO: 15 comprise or consists of substitutions at at least the group of codons consisting of codons number 109, 110, 111, 112, 113, 114, 115, 116, 119, 120, 122, 123, 252, 255, 256, 257, 258, 259, 260, 261, 263, 264, 267, 268, 270, 369, 370, 372, 374, 375, 376, 377, 378, 379, 381, 382, 542, 543, 545, 546, 547, 548, 549, 550, 552, 554, 555, 557, 559, 560, 704, 705, 707, 708, 709, 713, 718, 742, 743, 746, 747, 748, 749, 751, 759, 760, 761, 762, 765, 766, 767, 769, 771, 772, and 773.

In some embodiments, the synonymous substitutions within SEQ ID NO: 15 comprise or consist of the group of mutations consisting of: A325T, G326C, T327C, C330G, A333C, T336A, T339G, C342G, C343T, C345G, A348C, A357C, C360T, A366C, G369C, C756T, T765C, C768A, C771T, G774T, T775C, G777C, A780T, A783T, C789T, C792T, T801A, T802C, G804T, C810T, A1107G, C1110T, T1116C, G1122A, A1125G, A1128T, C1131T, T1134C, A1137G, C1143T, C1146T, A1624C, G1626C, A1629G, C1635G, A1638G, T1641G, A1644C, G1647C, A1648T, G1649C, T1650C, A1656G, A1662C, T1665G, G1671C, T1677G, T1680C, T2112A, G2115T, T2121A, C2124A, G2127A, C2139T, C2154T, C2226A, C2229T, T2236C, G2238T, T2241C, A2244C, A2247C, C2253T, A2277G, G2280C, T2281C, G2283T, G2286T, T2293C, G2295A, T2298C, G2301C, A2307T, T2313C, A2316G, and T2319C. In some embodiments, the region after synonymous substitution comprises SEQ ID NO: 20. In some embodiments, the region after synonymous substitution consists of SEQ ID NO: 20.

In some embodiments, the synonymous substitutions within SEQ ID NO: 15 comprise or consists of substitutions at at least the group of codons consisting of codons number 19, 29, 30, 33, 36, 47, 62, 64, 104, 114, 128, 136, 178, 182, 198, 201, 202, 218, 235, 242, 246, 247, 251, 252, 258, 267, 280, 295, 296, 311, 313, 314, 315, 332, 336, 357, 372, 374, 384, 385, 386, 387, 389, 396, 404, 425, 469, 477, 484, 485, 499, 502, 510, 522, 545, 546, 558, 563, 574, 583, 589, 590, 592, 596, 615, 625, 638, 641, 648, 666, 668, 680, 681, 727, 730, 731, 738, 745, 746, 759, 760, 765, 766, 775, 780, 783, 787.

In some embodiments, the synonymous substitutions within SEQ ID NO: 15 comprise or consist of the group of mutations consisting of: C55A, C57G, C87G, T88C, G99C, A108G, C141T, T184A, C185G, A186C, T192G, C310A, A312G, C342G, C382A, T384G, T406C, G408C, G534C, A546G, T594G, A603G, C606T, G654A, T703C, G705C, G726A, A738G, C741G, A753C, C756T, G774A, T801C, C840T, A885G, C888G, T933G, T939G, C942T, T943C, G945C, C996T, C1008T, C1071T, T1116C, G1122A, A1152G, G1155T, A1158G, G1161T, A1167G, A1188G, G1212T, G1275C, C1407A, A1431G, A1452T, C1455G, G1497A, T1504C, G1506C, C1530T, A1566G, C1635G, A1638T, C1674A, G1689C, G1722C, G1749C, A1767G, T1768C, G1770C, C1776G, T1786A, C1787G, A1845T, A1875T, C1914T, T1923A, T1942C, G1944C, G1998C, T2004C, A2040G, C2043G, T2181A, T2188A, C2189G, A2190C, T2191C, T2214C, T2233A, C2234G, A2235C, T2236C, A2277G, G2280T, T2293C, G2295C, T2298G, T2323A, C2324G, C2340A, A2349G, and C2361T. In some embodiments, the region after synonymous substitution comprises SEQ ID NO: 21. In some embodiments, the region after synonymous substitution consists of SEQ ID NO: 21.

In some embodiments, the region encodes a Zika virus NS5 protein and comprises a sequence selected from SEQ ID NO: 3-6, 8-11 and 13. In some embodiments, the region encodes a Zika virus NS5 protein and consists of a sequence selected from SEQ ID NO: 3-6, 8-11 and 13. In some embodiments, the region encodes a Zika virus E protein, C protein and prM protein and comprises a sequence selected from SEQ ID NO: 17-21. In some embodiments, the region encodes a Zika virus E protein, C protein and prM protein and consists of a sequence selected from SEQ ID NO: 17-21. In some embodiments, the attenuated virus comprises any one of SEQ ID NO: 3-6, 8-11, 13 and 17-21. In some embodiments, the attenuated virus comprises at least two of SEQ ID NO: 3-6, 8-11, 13 and 17-21. In some embodiments, the attenuated virus comprises at least one of SEQ ID NO: 3-6, 8-11, and 13 and at least one of SEQ ID NO: 17-21.

The full Hepatitis C virus genome can be found on NCBI which provides numerous individual HCV virus isolates. In some embodiments, the Hepatitis C virus genome is a hybrid genome. In some embodiments, the hybrid is a hybrid of two HCV isolates. In some embodiments, the isolates are H77C and JFH1. The H77C/JFH1 recombinant genome can be found at ncbi.nlm.nih.gov/nuccore/165873794. In some embodiments, the HPV genome is provided in SEQ ID NO: 34. The genome contains approximately 9450 nucleotides. In some embodiments, the nucleotide sequence encoding the WT HCV NS5 protein is provided in SEQ ID NO: 22. In some embodiments, the region before synonymous substitution comprises SEQ ID NO: 22. In some embodiments, the region before synonymous substitution consists of SEQ ID NO: 22. In some embodiments, SEQ ID NO: 22 provides nucleotides 6257 to 9427 of the HCV genome. In some embodiments, the HPV genome is provided in SEQ ID NO: 34. In some embodiments, SEQ ID NO: 22 provides the 3′ end of the HCV genome coding region. In some embodiments, the Zika genome encodes a polyprotein comprising/consisting of the amino acid sequence provided in SEQ ID NO: 35. In some embodiments, SEQ ID NO: 34 encodes SEQ ID NO: 35.

In some embodiments, SEQ ID NO: 22 encodes the C-terminus of the HCV polyprotein. In some embodiments, SEQ ID NO: 22 encodes the HCV NS5 protein. In some embodiments, SEQ ID NO: 22 encodes the amino acid sequence provided in SEQ ID NO: 23. In some embodiments, the region encodes SEQ ID NO: 23. In some embodiments, the region both before and after synonymous codon substitution encodes SEQ ID NO: 23.

In some embodiments, the synonymous substitutions within SEQ ID NO: 22 comprise or consists of substitutions at at least the group of codons consisting of codons number 1, 43, 106, 127, 169, 190, 211, 232, 266, 286, 327, 368, 388, 430, 468, 489, 510, 531, 552, 573, 594, 615, 657, 678, 762, 783, 804, 825, 846, 909, 951, and 993. It will be understood that the codon numbering is with respect to SEQ ID NO: 22, wherein the first 3 nucleotides are codon 1, nucleotides 4-6 are codon 2, nucleotides 7-9 are codon 3 and so on. It will be understood that many amino acids are encoded by more than 2 codons and so there may be multiple possibilities for synonymous substitution of the recited codons.

In some embodiments, the synonymous substitutions within SEQ ID NO: 22 comprise or consist of the group of mutations consisting of: C3T, C144G, C345G, C408T, G534A, T595C, G597T, A663C, C726G, G831C, C894T, C1026A, T1149A, T1209G, C1338T, G1458A, A1521C, C1584T, C1647G, G1710T, A1791G, T1857G, C1920T, T2055C, C2118T, A2370T, T2445A, G2517C, C2578A, G2580A, C2652A, T2856C, G2997A, and A3159T. It will be understood that the numbering of the nucleotides is with respect to SEQ ID NO: 22. Further, the notation of “C3T” means that the cytosine at position 3 in the sequence is mutated to thymine. These mutations are given for a DNA genome of the virus. If the genome is an RNA genome, then the thymines will be replaced with uracils. In some embodiments, the region after synonymous substitution comprises SEQ ID NO: 24. In some embodiments, the region after synonymous substitution consists of SEQ ID NO: 24.

In some embodiments, the synonymous substitutions within SEQ ID NO: 22 comprise or consists of substitutions at at least the group of codons consisting of codons number 9, 16, 23, 30, 37, 44, 51, 58, 65, 79, 86, 100, 107, 114, 121, 135, 142, 149, 156, 163, 170, 198, 205, 212, 233, 243, 250, 253, 260, 267, 280, 300, 307, 314, 321, 328, 341, 348, 362, 383, 389, 396, 410, 417, 424, 431, 438, 448, 455, 469, 476, 483, 490, 497, 504, 532, 539, 546, 560, 567, 574, 581, 602, 609, 623, 630, 637, 644, 651, 665, 672, 693, 707, 721, 728, 749, 763, 770, 777, 784, 791, 798, 805, 812, 819, 826, 833, 840, 847, 854, 875, 882, 889, 896, 903, 910, 917, 931, 945, 952, 966, 973, 987, and 994.

In some embodiments, the synonymous substitutions within SEQ ID NO: 22 comprise or consist of the group of mutations consisting of: T36C, C57T, T79C, C102T, G123A, C147T, C168G, C189T, C210A, G258C, C279T, C324G, G348T, G369A, A390C, A432G, T451A, C452G, C474T, A495G, G516A, T537G, C624T, G645C, A666C, C729T, C759T, C780T, T792C, G813A, C834T, C876G, A939G, G960T, A984C, T1008C, C1029G, A1068T, C1089T, C1131T, T1194C, G1212A, T1233C, G1278T, A1299G, G1320A, T1341G, T1365A, C1395T, C1419T, T1459C, G1461T, C1482T, C1503T, A1524T, T1545C, A1564C, G1566C, C1650T, T1669C, G1671T, A1690C, A1692G, C1734T, G1764C, A1794C, C1818G, T1881C, C1900A, T1944C, C1968T, T1989A, C2010T, G2034A, C2079T, G2100A, G2163C, A2203T, G2204C, G2247A, T2268C, C2331T, A2373G, G2397A, C2424G, T2448A, A2469G, C2499T, T2518C, G2520T, A2541G, A2562G, T2583C, T2610G, C2634A, A2655C, C2682T, A2751G, C2772T, A2791C, T2814C, C2838T, A2859T, C2886G, C2931G, C2976T, C3000T, C3051T, T3078C, T3135C, and C3162T. In some embodiments, the region after synonymous substitution comprises SEQ ID NO: 25. In some embodiments, the region after synonymous substitution consists of SEQ ID NO: 25.

In some embodiments, the synonymous substitutions within SEQ ID NO: 22 comprise or consists of substitutions at at least the group of codons consisting of codons number 132, 133, 138, 139, 140, 141, 145, 146, 203, 204, 206, 208, 209, 213, 214, 215, 216, 217, 486, 488, 490, 491, 493, 494, 496, 497, 498, and 500.

In some embodiments, the synonymous substitutions within SEQ ID NO: 22 comprise or consist of the group of mutations consisting of: A396G, C397T, G399A, G414T, A417G, T420C, T423A, T435G, T438G, A609C, A612C, G618A, C624T, C627A, T639G, G642A, G645A, C646A, C649A, C651G, G1458A, A1464C, C1470T, T1473G, T1479C, C1482T, G1488C, T1489C, A1494C, and T1500C. In some embodiments, the region after synonymous substitution comprises SEQ ID NO: 26. In some embodiments, the region after synonymous substitution consists of SEQ ID NO: 26.

In some embodiments, the synonymous substitutions within SEQ ID NO: 22 comprise or consists of substitutions at at least the group of codons consisting of codons number 105, 106, 107, 109, 110, 112, 113, 114, 115, 116, 117, 132, 133, 138, 139, 140, 141, 145, 146, 203, 204, 206, 208, 209, 213, 214, 215, 216, 217, 333, 334, 335, 337, 338, 339, 340, 341, 342, 343, 345, 346, 347, 349, 366, 368, 371, 372, 373, 376, 377, 378, 379, 380, 486, 488, 490, 491, 493, 494, 496, 497, 498, 500, 620, 621, 625, 627, 628, 629, 631, 632, 633, 723, 727, 728, 729, 730, 733, 734, 737, 751, 753, 757, 762, 764, 766, 836, 837, 838, 839, 840, 842, 845, 846, 848, 850, 981, 983, 984, 985, 987, 989, 990, 992, and 993.

In some embodiments, the synonymous substitutions within SEQ ID NO: 22 comprise or consist of the group of mutations consisting of: C315T, C318T, G321A, C327G, C330T, A334C, G339A, G342C, C345T, G348A, G351A, A396G, C397T, G399A, G414T, A417G, T420C, T423A, T435G, T438G, A609C, A612C, G618A, C624T, C627A, T639G, G642A, G645A, C646A, C649A, C651G, C999T, C1002T, T1005C, T1011C, T1014C, T1017C, C1018T, C1020G, C1023G, C1026T, C1029A, G1035A, C1038A, G1041A, T1047A, A1098G, C1102T, C1104G, G1113T, C1116T, C1119T, T1128C, C1131T, G1134A, C1137G, C1140G, G1458A, A1464C, C1470T, T1473G, T1479C, C1482T, G1488C, T1489C, A1494C, T1500C, G1860A, A1863G, C1875T, T1881G, C1884T, T1887C, C1891T, C1893G, C1896T, C1899T, T2169C, C2181T, A2184G, A2187G, G2190T, C2199T, C2202T, T2211G, A2253G, T2259C, C2271T, T2286C, A2292G, C2296T, G2508C, T2511C, G2514C, G2517C, T2518C, G2526A, C2533A, C2535A, C2536A, C2538G, C2544T, G2550C, A2943G, A2949G, G2952C, C2955T, C2961T, A2967G, T2970C, C2976T, and T2979C. In some embodiments, the region after synonymous substitution comprises SEQ ID NO: 27. In some embodiments, the region after synonymous substitution consists of SEQ ID NO: 27.

In some embodiments, the synonymous substitutions within SEQ ID NO: 22 comprise or consists of substitutions at at least the group of codons consisting of codons number 105, 106, 107, 109, 110, 113, 114, 115, 116, 117, 120, 132, 133, 139, 140, 141, 143, 145, 146, 203, 204, 206, 209, 212, 213, 214, 215, 216, 217, 333, 334, 335, 337, 338, 339, 340, 341, 342, 343, 344, 345, 346, 349, 366, 368, 369, 370, 371, 372, 376, 378, 379, 380, 486, 489, 491, 492, 493, 496, 497, 498, 499, 500, 621, 625, 627, 628, 629, 630, 631, 632, 633, 725, 728, 729, 730, 731, 733, 735, 737, 751, 756, 760, 762, 763, 764, 837, 838, 839, 840, 842, 844, 845, 846, 847, 850, 983, 984, 986, 987, 989, 990, 992, 993, and 995.

In some embodiments, the synonymous substitutions within SEQ ID NO: 22 comprise or consist of the group of mutations consisting of: C315T, C318T, G321A, C327G, C330T, G339A, G342T, C345T, G348A, G351A, G360A, A396G, C397T, A417G, T420C, T423G, A429G, T435G, T438G, A609C, A612C, G618C, C627A, G636A, T639G, G642T, G645C, G648T, C649A, C651G, C999A, C1002T, T1005G, T1011C, T1014C, T1017C, C1018T, C1020G, C1023G, C1026T, C1029A, G1032A, G1035A, C1038G, T1047A, A1098G, C1104G, G1107A, A1110G, G1113C, C1116T, T1128C, G1134A, C1137G, C1140T, G1458A, C1467T, T1473C, T1474C, T1479C, G1488C, T1489C, A1494T, C1497T, T1500C, A1863G, C1875T, T1881A, C1884T, T1887A, C1890T, C1893G, C1896A, C1899G, A2173C, A2175G, A2184T, A2187G, G2190C, C2193T, C2199T, A2203T, G2204C, C2205A, T2211G, A2253G, T2268C, A2280T, T2286C, G2289A, A2292G, T2511C, G2514C, G2517C, T2518C, G2526T, C2532A, C2533A, C2535A, C2536A, C2538A, A2539C, A2541G, G2550C, A2949G, G2952T, T2958A, C2961T, C2965A, T2970C, C2976T, T2979C, and G2985A. In some embodiments, the region after synonymous substitution comprises SEQ ID NO: 28. In some embodiments, the region after synonymous substitution consists of SEQ ID NO: 28.

In some embodiments, the synonymous substitutions within SEQ ID NO: 22 comprise or consists of substitutions at at least the group of codons consisting of codons number 105, 106, 107, 109, 110, 113, 114, 115, 116, 117, 120, 132, 138, 139, 141, 143, 144, 145, 146, 203, 206, 209, 210, 211, 213, 214, 215, 216, 217, 333, 334, 335, 336, 337, 339, 340, 341, 342, 343, 344, 346, 347, 349, 368, 369, 371, 373, 374, 376, 377, 378, 379, 380, 486, 487, 492, 493, 495, 496, 497, 498, 499, 500, 621, 625, 627, 628, 629, 630, 631, 632, 633, 723, 725, 726, 728, 729, 730, 734, 737, 750, 751, 753, 760, 762, 763, 837, 838, 840, 841, 842, 843, 844, 845, 846, 850, 982, 984, 985, 986, 987, 989, 990, 992, and 995.

In some embodiments, the synonymous substitutions within SEQ ID NO: 22 comprise or consist of the group of mutations consisting of: C315T, C318T, G321A, C327G, C330T, G339A, G342C, C345A, G348A, G351A, G360A, A396C, G414T, A417G, T423G, A429G, A432G, T435A, T438G, A609C, G618A, C627T, G630A, G633A, T639G, G642A, G645T, G648T, C649A, C651G, C999T, C1002T, T1005G, T1008G, T1011G, T1017C, C1018T, C1020G, C1023A, C1026T, C1029A, G1032A, C1038G, G1041A, T1047C, C1102T, C1104G, G1107A, G1113C, C1119T, G1122A, T1128C, C1131T, G1134A, C1137A, C1140A, G1458A, T1459C, T1474C, T1479C, G1485T, G1488C, T1489C, G1491A, A1494T, C1497T, T1500C, A1863G, C1875T, T1881A, C1884T, T1887C, C1890T, C1891T, C1893G, C1896A, C1899T, T2169C, A2173C, A2175G, T2178C, A2184T, A2187G, G2190C, C2202T, T2211G, G2250A, A2253G, T2259C, A2280T, T2286C, G2289A, T2511C, G2514C, T2518C, C2523T, G2526T, C2527A, G2529A, C2532A, C2533A, C2535A, C2536A, C2538A, G2550C, G2946C, G2952T, C2955T, T2958C, C2961T, A2967G, T2970C, C2976T, and G2985A. In some embodiments, the region after synonymous substitution comprises SEQ ID NO: 29. In some embodiments, the region after synonymous substitution consists of SEQ ID NO: 29.

In some embodiments, the synonymous substitutions within SEQ ID NO: 22 comprise or consists of substitutions at at least the group of codons consisting of codons number 12, 14, 21, 25, 46, 59, 60, 91, 95, 109, 126, 163, 224, 225, 241, 242, 282, 293, 303, 319, 320, 343, 347, 349, 350, 380, 400, 407, 415, 440, 457, 460, 461, 464, 467, 469, 478, 479, 480, 507, 508, 530, 559, 560, 608, 617, 642, 655, 663, 671, 686, 725, 736, 737, 741, 777, 784, 793, 798, 820, 824, 834, 856, 870, 885, 890, 898, 902, 903, 911, 965, 975, 986, 993, and 1053.

In some embodiments, the synonymous substitutions within SEQ ID NO: 22 comprise or consist of the group of mutations consisting of: T36C, C42G, T63C, T75C, G138C, C177T, C180G, T273C, G285A, C327G, G378T, C489G, A672C, T675C, C723G, C726A, A846G, A879C, C909T, G957C, G960A, C1029A, G1041A, T1047C, C1050A, C1140A, G1200C, C1221A, T1245C, G1320A, T1369A, C1370G, C1380T, T1383C, G1392C, C1401G, A1407T, T1434C, C1437A, T1440C, A1521C, A1524C, T1590C, A1677C, C1680A, T1824C, G1851A, T1926C, T1965C, T1989A, T2011C, G2013C, T2058C, A2173C, A2175G, G2208A, T2211G, T2223C, C2331G, T2352C, G2379A, C2394T, C2460A, T2472C, A2502C, T2568C, T2610A, A2655C, C2670T, T2694C, A2706G, C2709T, T2733C, G2895C, G2925C, T2958C, T2979C, and A3159C. In some embodiments, the region after synonymous substitution comprises SEQ ID NO: 30. In some embodiments, the region after synonymous substitution consists of SEQ ID NO: 30.

In some embodiments, the region comprises a nucleotide sequence of any one of SEQ ID NO: 24-30. In some embodiments, the attenuated virus comprises any one of SEQ ID NO: 24-30. In some embodiments, the region encoding a HCV NS5 protein comprises any one of SEQ ID NO: 24-30. In some embodiments, the region consists of a nucleotide sequence of any one of SEQ ID NO: 24-30. In some embodiments, the attenuated virus comprises any one of SEQ ID NO: 24-30. In some embodiments, the region encoding a HCV NS5 protein consists of any one of SEQ ID NO: 24-30. In some embodiments, a HCV NS5 comprises NS5A and NS5B.

In some embodiments, the synonymous substitutions within SEQ ID NO: 22 do not comprise or consists of substitutions at at least the group of codons consisting of codons number 22:10, 13, 18, 19, 35, 36, 42, 51, 57, 59, 60, 65, 67, 74, 80, 82, 89, 90, 94, 96, 100, 105, 106, 109, 113, 118, 119, 120, 136, 142, 148, 150, 154, 156, 170, 172, 175, 179, 185, 187, 192, 193, 194, 195, 201, 206, 208, 211, 212, 221, 223, 227, 228, 232, 233, 240, 243, 251, 257, 258, 259, 262, 266, 269, 271, 273, 278, 280, 291, 293, 297, 310, 315, 316, 317, 318, 330, 342, 352, 374, 408, 409, 415, 417, 418, 420, 421, 422, 424, 427, 441, 463, 465, 466, 470, 473, 474, 484, 486, 489, 494, 501, 504, 517, 521, 527, 535, 536, 538, 541, 549, 552, 553, 555, 557, 568, 569, 570, 571, 572, 573, 574, 580, 581, 584, 585, 587, 588, 591, 593, 595, 598, 603, 604, 609, 611, 617, 623, 624, 626, 628, 630, 635, 636, 637, 640, 643, 651, 659, 660, 661, 666, 677, 687, 689, 690, 691, 694, 696, 698, 699, 702, 706, 707, 709, 715, 722, 724, 726, 727, 729, 731, 734, 740, 745, 746, 748, 754, 757, 758, 761, 765, 768, 774, 776, 785, 788, 789, 792, 797, 798, 803, 805, 806, 807, 808, 810, 811, 812, 814, 815, 816, 822, 825, 827, 829, 830, 831, 833, 834, 836, 838, 841, 848, 849, 851, 853, 861, 862, 865, 868, 869, 870, 873, 876, 877, 879, 894, 895, 896, 905, 915, 918, 919, 922, 924, 925, 928, 933, 937, 939, 941, 943, 944, 945, 948, 949, 950, 951, 955, 956, 958, 959, 962, 963, 964, 967, 969, 970, 973, 975, 979, 981, 982, 987, 988, 991, 992, 995, 996, 998, 999, 1002, 1012, 1014, 1017, 1020, 1022, 1023, 1024, 1025, 1028, 1029, 1031, 1033, 1035, 1036, 1041, 1042, 1043, 1044, 1045, 1048, 1050, 1051, 1054, 1055, and 1057. In some embodiments, the region will be devoid of a combination of synonymous substitutions at the above recited codons. It will be understood that the codon numbering is with respect to SEQ ID NO: 22, wherein the first 3 nucleotides are codon 1, nucleotides 4-6 are codon 2, nucleotides 7-9 are codon 3 and so on. It will be understood that many amino acids are encoded by more than 2 codons and so there may be multiple possibilities for synonymous substitution of the recited codons.

In some embodiments, the region does not comprise SEQ ID NO: 31. In some embodiments, the region is devoid of SEQ ID NO: 31. In some embodiments, the modified genome is devoid of SEQ ID NO: 31.

According to another aspect, there is provide a cell comprising a modified genome of the invention.

According to another aspect, there is provide a composition comprising a modified genome of the invention.

According to another aspect, there is provide a composition comprising a modified cell of the invention.

According to another aspect, there is provide a composition comprising an attenuated virus of the invention.

In some embodiments, the attenuated virus is an attenuated form of a virulent virus of the invention. In some embodiments, the composition is a pharmaceutical composition. In some embodiments, the composition is a vaccine composition. In some embodiments, a vaccine composition is a vaccine. In some embodiments, the composition is formulated to administer to a subject. In some embodiments, the composition is formulated for systemic administration. In some embodiments, the composition is formulated to induce an immune response in a subject. In some embodiments, the immune response is a protective immune response. In some embodiments, protective is protective against the virulent virus. In some embodiments, the attenuated virus and/or vaccine composition induces an immune response in a host animal sufficient to provide protection from the virulent virus. In some embodiments, protection comprises reduced infection. In some embodiments, reduced infection comprises a reduced risk of infection. In some embodiments, protection comprises reduced mortality. In some embodiment, reduced mortality is reduced risk of dying. In some embodiments, protection comprises a reduction in a symptom. In some embodiments, protection comprises reduced symptoms. In some embodiments, protection comprises an increased chance of asymptomatic infection. In some embodiments, protection comprises a reduced viral load. In some embodiments, viral load is viral load in the subject.

In some embodiments, the composition further comprises a pharmaceutically acceptable carrier, excipient or adjuvant. In some embodiments, the composition further comprises an adjuvant. In some embodiments, the adjuvant is a vaccine adjuvant. It should be understood that an attenuated virus of the invention, where used to elicit a protective immune response (i.e., immunize) in a subject or to prevent a subject from becoming afflicted with a virus-associated disease, is administered to the subject in the form of a composition additionally comprising a pharmaceutically acceptable carrier.

Vaccine adjuvants are well known in the art and any known adjuvant that enhances an immune response induced by the vaccine may be used. Examples of vaccine adjuvants include, but are not limited to aluminum adjuvants (e.g., aluminum salts such as amorphous aluminum hydroxyphosphate sulfate (AAHS), aluminum hydroxide, aluminum phosphate, and potassium aluminum sulfate (Alum)), AS01B, AS04, CpG1018, Matrix-M and MF59. In some embodiments, the vaccine comprises an aluminum adjuvant.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In another aspect, the present invention provides a method for eliciting a protective immune response in a subject comprising administering to the subject a prophylactically or therapeutically effective dose of any of the vaccine compositions of the invention, thereby eliciting a protective immune response in a subject.

This invention also provides a method for preventing a subject from becoming afflicted with a virus-associated disease comprising administering to the subject a prophylactically effective dose of any of the instant vaccine compositions. In embodiments of the above methods, the subject has been exposed to a pathogenic virus. “Exposed” to a pathogenic virus means contact with the virus such that infection could result.

The invention further provides a method for delaying the onset, or slowing the rate of progression, of a virus-associated disease in a virus-infected subject comprising administering to the subject a therapeutically effective dose of any of the instant vaccine compositions.

As used herein, “administering” means delivering using any of the various methods and delivery systems known to those skilled in the art. Administering can be performed, for example, intraperitoneally, intracerebrally, intravenously, orally, transmucosally, subcutaneously, transdermally, intradermally, intramuscularly, topically, parenterally, via implant, intrathecally, intralymphatically, intralesionally, pericardially, or epidurally. An agent or composition may also be administered in an aerosol, such as for pulmonary and/or intranasal delivery. Administering may be performed, for example, once, a plurality of times, and/or over one or more extended periods.

In some embodiments, eliciting a protective immune response comprises immunizing. In some embodiments, eliciting a protective immune response comprises vaccinating. Eliciting a protective immune response in a subject can be accomplished, for example, by administering a primary dose of a vaccine to a subject, followed after a suitable period of time by one or more subsequent administrations of the vaccine. A suitable period of time between administrations of the vaccine may readily be determined by one skilled in the art and is usually on the order of several weeks to months. The present invention is not limited, however, to any particular method, route or frequency of administration.

A “subject” refers to any animal or artificially modified animal. Animals include, but are not limited to, humans, non-human primates, cows, horses, sheep, pigs, dogs, cats, rabbits, ferrets, rodents such as mice, rats and guinea pigs, and birds. In some embodiments, the subject is a mammal. In some embodiments, the subject is a human. In some embodiments, a subject is a subject in need of vaccination. In some embodiments, the subject is a subject at risk for infection by the virulent virus.

A “prophylactically effective dose” is any amount of a vaccine that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood of the disorder's onset in the subject, by at least two-fold, preferably at least tenfold. For example, if a subject has a 1% chance of becoming infected with a virus, a two-fold reduction in the likelihood of the subject becoming infected with the virus would result in the subject having a 0.5% chance of becoming infected with the virus. Most preferably, a “prophylactically effective dose” induces in the subject an immune response that completely prevents the subject from becoming infected by the virus or prevents the onset of the disorder in the subject entirely.

As used herein, a “therapeutically effective dose” is any amount of a vaccine that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

The dosage administered will be dependent upon the age, health, and weight of the recipient, kind of concurrent treatment, if any, frequency of treatment, and the nature of the effect desired.

Certain embodiments of any of the instant immunization and therapeutic methods further comprise administering to the subject at least one adjuvant. An “adjuvant” shall mean any agent suitable for enhancing the immunogenicity of an antigen and boosting an immune response in a subject. Numerous adjuvants, including particulate adjuvants, suitable for use with both protein- and nucleic acid-based vaccines, and methods of combining adjuvants with antigens, are well known to those skilled in the art. Suitable adjuvants for nucleic acid based vaccines include, but are not limited to, Quil A, imiquimod, resiquimod, and interleukin-12 delivered in purified protein or nucleic acid form. Adjuvants suitable for use with protein immunization include, but are not limited to, alum, Freund's incomplete adjuvant (FIA), saponin, Quil A, and QS-21.

The invention also provides a kit for immunization of a subject with an attenuated virus of the invention. The kit comprises the attenuated virus, a pharmaceutically acceptable carrier, an applicator, and an instructional material for the use thereof. More than one virus may be preferred where it is desirable to immunize a host against a number of different isolates of a particular virus. The invention includes other embodiments of kits that are known to those skilled in the art. The instructions can provide any information that is useful for directing the administration of the attenuated viruses.

According to another aspect, there is provided an attenuated HCV variant comprising synonymous mutations.

According to another aspect, there is provided a method for designing an attenuated Hepatitis C Virus (HCV) variant based on mRNA folding, comprising:
a. obtaining a multiple sequence alignment (MSA) of HCV strains;
b. using computational algorithms to identify regions within the HCV genome with significant selection for strong or weak RNA folding;
c. introducing a plurality of synonymous mutations in the identified regions to alter the local folding energy (LFE) by at least 15%, thereby disrupting the RNA secondary structures critical for the viral life cycle, while preserving the amino acid sequence of the viral proteins;
d. generating the attenuated HCV variant with the introduced synonymous mutations;
e. evaluating the viral fitness of the attenuated HCV variant; and
f. selecting a variant with reduced fitness.

According to another aspect, there is provided a method for designing an attenuated Hepatitis C Virus (HCV) variant based on underrepresented sequences, comprising:
a. obtaining a multiple sequence alignment (MSA) of HCV strains;
b. using computational algorithms to identify underrepresented (UR) sequences within the HCV genome;
c. introducing a plurality of synonymous mutations to insert the identified UR sequences into their corresponding regions, thereby disrupting the RNA regulatory elements critical for the viral life cycle, while preserving the amino acid sequence of the viral proteins;
d. generating the attenuated HCV variant with the introduced synonymous mutations;
e. evaluating the viral fitness of the attenuated HCV variant; and
f. selecting a variant with reduced fitness.

According to another aspect, there is provided a method for designing an attenuated Hepatitis C Virus (HCV) variant based on evolutionary conservation, comprising:
a. obtaining a multiple sequence alignment (MSA) of HCV strains;
b. calculating the nucleotide Shannon entropy score for each column in the MSA to assess the evolutionary conservation of each genomic position;
c. introducing a plurality of synonymous mutations in positions with medium-to-high conservation to synonymous codons with lower relative frequencies, thereby disrupting the RNA regulatory elements critical for the viral life cycle, while preserving the amino acid sequence of the viral proteins;
d. generating the attenuated HCV variant with the introduced synonymous mutations;
e. evaluating the viral fitness of the attenuated HCV variant; and
f. selecting a variant with reduced fitness.

In some embodiments, viral fitness is measured by at least one of RNA levels, infection percentage, and/or viral spread in liver cells. In some embodiments, the method further comprises confirming the genomic stability of the attenuated HCV variant over time without significant reversion to the wild-type sequence. In some embodiments, the method comprises selecting a variant confirmed to have genomic stability over time.

In one embodiment, the attenuated Hepatitis C Virus (HCV) embodiment comprises a series of synonymous mutations specifically introduced into the NS5A gene of the HCV genome. These mutations are designed to disrupt the RNA secondary structures and regulatory sequences necessary for the viral life cycle, while preserving the amino acid sequence of the viral proteins. This embodiment ensures that the virus exhibits reduced viral fitness, characterized by lower RNA levels, reduced infection percentage, and smaller viral spread in liver cells. In another embodiment, the synonymous mutations are introduced into the NS5B gene, targeting regions with high selection for strong RNA folding to alter the local folding energy (LFE) by at least 15%. This modification further attenuates the virus by disrupting necessary RNA structures without altering the protein sequence. Additionally, an embodiment may involve introducing synonymous mutations in regions with underrepresented sequences, thereby inserting these sequences into their corresponding regions to disrupt RNA regulatory elements. This approach can be applied to both the NS5A and NS5B genes, ensuring a comprehensive attenuation strategy. Furthermore, the attenuated HCV embodiment can be used as a vaccine candidate, leveraging the reduced pathogenicity and enhanced genetic stability to provide immunity against HCV. Each of these embodiments maintains the principle of using synonymous mutations to disrupt RNA structures while preserving the protein sequence, ensuring the virus remains attenuated and genetically stable over time.

In one embodiment, the method for designing an attenuated Hepatitis C Virus (HCV) variant based on mRNA folding involves obtaining a multiple sequence alignment (MSA) of HCV strains from a database comprising at least 699 complete HCV strains. This ensures a comprehensive representation of the viral genome for accurate identification of regions with significant selection for strong or weak RNA folding.

In another embodiment, the computational algorithms used to identify regions with significant selection for strong or weak RNA folding include a sliding window approach with a window length of 39 nucleotides. This approach allows for precise detection of local folding energy (LFE) variations across the viral genome.

In a further embodiment, the synonymous mutations are introduced only in codons whose frequency in the MSA column is at least 10%. This ensures that the introduced mutations are not rare and are evolutionarily relevant, thereby maintaining the stability of the attenuated virus.

In yet another embodiment, the evaluation of viral fitness includes measuring the size of viral foci in liver cells. This provides a quantitative assessment of the virus's ability to spread and infect cells, which is a critical indicator of viral fitness.

In an additional embodiment, the evaluation of viral fitness includes measuring the percentage of infected liver cells over a period of 4 weeks. This long-term assessment helps in understanding the sustained impact of the introduced mutations on viral fitness.

In a further embodiment, the confirmation of genomic stability includes deep sequencing of the NS5A and NS5B genes of the attenuated HCV variant. This ensures that the introduced synonymous mutations remain stable over time without significant reversion to the wild-type sequence.

In another embodiment, the synonymous mutations are designed to change the local folding energy (LFE) by at least 15% in regions with significant selection for strong RNA folding. This targeted approach ensures that the critical RNA secondary structures are effectively disrupted, leading to reduced viral fitness.

In yet another embodiment, the synonymous mutations are designed to change the local folding energy (LFE) by at least 15% in regions with significant selection for weak RNA folding. This ensures a comprehensive disruption of RNA structures that are critical for the viral life cycle.

In one embodiment, the method for designing an attenuated Hepatitis C Virus (HCV) variant based on underrepresented sequences involves obtaining a multiple sequence alignment (MSA) of HCV strains from a database comprising at least 699 complete HCV strains. This ensures a comprehensive representation of the viral genome for accurate identification of underrepresented sequences.

In another embodiment, the computational algorithms used to identify underrepresented (UR) sequences include synonymous codon permutations and synonymous dinucleotide permutations. This approach allows for precise detection of UR sequences that may serve as adverse regulatory elements.

In a further embodiment, the synonymous mutations are introduced only in codons whose frequency in the MSA column is at least 10%. This ensures that the introduced mutations are not rare and are evolutionarily relevant, thereby maintaining the stability of the attenuated virus.

In yet another embodiment, the evaluation of viral fitness includes measuring the size of viral foci in liver cells. This provides a quantitative assessment of the virus's ability to spread and infect cells, which is a critical indicator of viral fitness.

In an additional embodiment, the evaluation of viral fitness includes measuring the percentage of infected liver cells over a period of 4 weeks. This long-term assessment helps in understanding the sustained impact of the introduced mutations on viral fitness.

In a further embodiment, the confirmation of genomic stability includes deep sequencing of the NS5A and NS5B genes of the attenuated HCV variant. This ensures that the introduced synonymous mutations remain stable over time without significant reversion to the wild-type sequence.

In another embodiment, the synonymous mutations are designed to insert underrepresented sequences in both frame 1 and frame 2 of the HCV genome. This comprehensive approach ensures that the critical RNA regulatory elements are effectively disrupted, leading to reduced viral fitness.

In yet another embodiment, the synonymous mutations are introduced in the NS5A gene. This targeted approach ensures that the critical RNA regulatory elements within the NS5A gene are disrupted, leading to reduced viral fitness.

In an additional embodiment, the synonymous mutations are introduced in the NS5B gene. This targeted approach ensures that the critical RNA regulatory elements within the NS5B gene are disrupted, leading to reduced viral fitness.

In one embodiment, the method for designing an attenuated Hepatitis C Virus (HCV) variant based on evolutionary conservation involves obtaining a multiple sequence alignment (MSA) of HCV strains from a database comprising at least 699 complete HCV strains. This ensures a comprehensive representation of the viral genome for accurate assessment of evolutionary conservation.

In another embodiment, the computational algorithms used to calculate the nucleotide Shannon entropy score include a sliding window approach with a window length of 39 nucleotides. This approach allows for precise detection of evolutionary conservation across the viral genome.

In a further embodiment, the synonymous mutations are introduced only in codons whose frequency in the MSA column is at least 10%. This ensures that the introduced mutations are not rare and are evolutionarily relevant, thereby maintaining the stability of the attenuated virus.

In yet another embodiment, the evaluation of viral fitness includes measuring the size of viral foci in liver cells. This provides a quantitative assessment of the virus's ability to spread and infect cells, which is a critical indicator of viral fitness.

In an additional embodiment, the evaluation of viral fitness includes measuring the percentage of infected liver cells over a period of 4 weeks. This long-term assessment helps in understanding the sustained impact of the introduced mutations on viral fitness.

In a further embodiment, the confirmation of genomic stability includes deep sequencing of the NS5A and NS5B genes of the attenuated HCV variant. This ensures that the introduced synonymous mutations remain stable over time without significant reversion to the wild-type sequence.

In another embodiment, the synonymous mutations are introduced in the NS5A gene. This targeted approach ensures that the critical RNA regulatory elements within the NS5A gene are disrupted, leading to reduced viral fitness.

In yet another embodiment, the synonymous mutations are introduced in the NS5B gene. This targeted approach ensures that the critical RNA regulatory elements within the NS5B gene are disrupted, leading to reduced viral fitness.

In an additional embodiment, the evaluation of viral fitness includes measuring RNA levels, infection percentage, and viral spread in liver cells over a period of 4 weeks. This comprehensive assessment provides a detailed understanding of the impact of the introduced mutations on viral fitness.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Animals: 85 male and female AG129 mice produced by an in-house colony were used. Mice were randomly assigned to experimental groups and individually marked with ear tags.

Virus: Zika virus (Malaysian strain, P6-740) was used at a challenge dose of ˜100 CCID₅₀ administered via subcutaneous (s.c.) injection in a volume of 0.1 mL.

Test agent: Synthetic viruses including a synthetic WT, 104-169, 104-169 MED_ENT_75_78, and 104-169 HI_ENT_75_76 were provided for testing in the mouse model. They were diluted in sterile PBS prior to use.

Quantification of viral RNA:RNA was extracted from serum samples (two pools per group) collected 3 and 7 dpi using the QIAamp MinElute Virus Spin Kit (QIAGEN, cat #57704) and was eluted with 25 μl of elution buffer. Tissue RNA was extracted using TriZol and total RNA was resuspended in 50 μl water. A volume of 2 μl of the RNA preparation was used for amplification using a one-step RNA amplification kit from Agilent (Brilliant II QRT-PCR Master Mix kit, cat #600809). Serial dilutions of synthetic RNA spanning the amplification region was used as a positive control and a standard curve was generated. Samples were subjected to 40 cycles of 15 sec at 95° C. and 60 sec at 60° C. following an initial single cycle of 30 min at 50° C. and 10 min at 95° C. Samples of unknown quantity were quantified by extrapolation of C(t) values using a curve generated from serial dilutions of synthetic ZIKV RNA.

Quantification of neutralizing antibody: Neutralizing antibody was quantified using a 50% plaque reduction neutralization titer (PRNT₅₀) assay. Serum samples were heat inactivated at 56° C. for 30 minutes in a water bath. One half serial dilutions, starting at a 1/10 dilution, of test sera were made. Dilutions were then mixed 1:1 with an appropriate titer of ZIKV in MEM containing 2% fetal bovine serum (FBS) and incubated at 4° C. overnight. The virus-serum mixture was then added to individual wells of a 12-well tissue culture plate with Vero76 cells (5e5 cells/well). Viral adsorption proceeded for one hour at 37° C. and 5% CO₂, followed by addition of 1.7% (4000 cps) methylcellulose overlay medium containing 10% FBS to each well. Plates were incubated for 4 days, then stained with crystal violet (with 1% (wt/vol) crystal violet in 10% (vol/vol) ethanol) containing 2% formalin for 20 minutes. The reciprocal of the dilution of test serum that resulted in ≥50% reduction in average plaques from virus control was recorded as the PRNT₅₀ value.

Experiment Design: Mice were vaccinated with WT synthetic, 104-169, 104-169 MED_ENT_75_78, or 104-169 HI_ENT_75_76 at doses of 10² or 10³ FFU in a volume of 0.1 mL. A single IP injection was administered-28 days post-virus inoculation (dpi). Animals were challenged with WT ZIKV via SC injection. Placebo and normal control groups were included. Animals were monitored daily for survival and disease signs including conjunctivitis, hunching, lethargy, and limb weakness or paralysis. Individual weights were recorded daily-28 to 28 dpi. Serum was collected from all animals −15 and −1 dpi for assessment of neutralizing Ab by PRNT assay. Serum was collected 3 and 7 dpi for virus titration by qRT-PCR.

Statistical analysis: Survival data were analyzed using the Wilcoxon log-rank survival analysis and all other statistical analyses were done using one-way ANOVA using a Bonferroni group comparison (Prism 5, GraphPad Software, Inc).

Ethics regulation of Laboratory animals: This study was conducted in accordance with the approval of the Institutional Animal Care and Use Committee of Utah State University (Protocol #10172). The work was done in the AAALAC-accredited Laboratory Animal Research Center of Utah State University. The U. S. Government (National Institutes of Health) approval was renewed 9 Mar. 2018 (PHS Assurance no. D16-00468) in accordance with the National Institutes of Health Guide for the Care and Use of Laboratory Animals (Revision; 2015).

### Example 1: Zika Virus (ZIKV) Synthetic Variants of the NS5 Protein Coding Region

The Zika genome encodes a polyprotein that includes all 10 Zika proteins. Variants of the region that encodes the NS5 protein, which are predicted to cause virus attenuation, were generated based on two approaches/measures (which are not mutually exclusive). The DNA sequence encoding the polyprotein containing NS5 is located at positions 7668-10376 in the Zika genome (SEQ ID NO: 1).

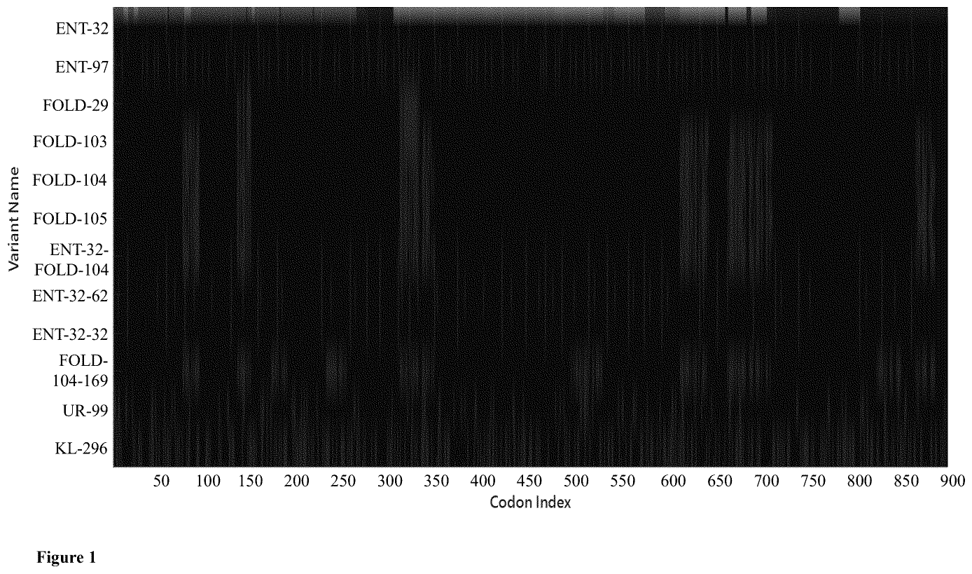

Entropy analysis/rare codon approach (2 variants): Methods of attenuating a virus by exchanging common codons for rare codons are disclosed in U.S. Pat. No. 11,236,344, herein incorporated by reference in its entirety. Of the 903 total codons within NS5 (SEQ ID NO: 2) 271 synonymous codons were identified that met the required criteria and a variant with 32 synonymous replacements (ENT_32, SEQ ID NO: 3) and one with 97 synonymous replacements (ENT_97, SEQ ID NO: 4) (uniform distribution, mutually exclusive mutations, see FIG. 1) were generated. The criteria for insertable codons were as follows: A codon to be inserted must appear in the evolution of Zika with a frequency of at least 15% (i.e., not a deleterious mutation). An inserted codon must be relatively rare in comparison to the actual codon found in Zika. Since the sets are mutually exclusive it is not clear which of the two variants is more attenuated.

Folding approach (4 variants): An algorithm that measures RNA folding and de-optimizes the local folding energy (not using rare codons) was used. This approach is disclosed in International Patent Publication WO2017056094, herein incorporated by reference in its entirety, and was used to generate a gradient of changes to the local folding energy (15%, 50%, and 100%) at 8 positions (windows of 39 nucleotides) that are detected to be functional (evolutionarily conserved strong folding at a pval of <0.05) (FIG. 1). Briefly, 10,000 randomizations for a given selected interval were generated and the local folding energy calculated. Codons were replaced with synonymous codons that are evolutionarily conserved (observed in at least 15% of genomes of the virus). Synonymous substitutions were generated until the desired total change in folding energy was achieved. The variant with a 15% reduction (FOLD_103, SEQ ID NO: 6) is predicted to be less attenuated than the variant with 50% reduction (FOLD_104, SEQ ID NO: 7), which is itself predicted to be less attenuated than the 100% reduction (FOLD_105, SEQ ID NO: 8). An additional variant was generated also with 15% reduction but only over part of the coding region (only two functional positions were altered) (FOLD_29, SEQ ID NO: 5). This variant was predicted to be the least attenuated.

Importantly, the variants were designed in order to show that the number of mutations are not the simple variable that predicts the effect on the virus. Further, based on the measure used, each of variants can be easily engineered to be further attenuated or to be less attenuated. The folding variants can be modulated by changing the folding and the entropy via changing the number of mutations and/or the evolutionary frequency of the replaced codon. Indeed, an additional six variants were generated based on the previous approaches (with enhancements) or using new design methods.

ENT_32_FOLD_104 (SEQ ID NO: 9) is a variant generated by merging two of the earlier variants, one bases on the entropy approach, and one based on folding. There was only one mutually mutated codon that was different between the two variants (found at codon number 332). The codon from the ENT_32 variant was used, as this variant is predicted to be weaker.

ENT_32_62 (SEQ ID NO: 10)—was generated with the entropy analysis. It contains the original 32 changes of ENT_32 and an additional 30 codon changes not presented in ENT 97. The mutations are relatively uniformly distributed across the sequence.

ENT_32_32 (SEQ ID NO: 11)—was generated with the entropy analysis. It contains changes in the same locations as in ENT_32 but inserts codons that are rarer than those used in ENT 32. However, the very rarest codons (i.e., with frequency lower than 5%) were not included. This resulted in 14 out of the 32 changed codons (44%) being different from ENT 32.

FOLD_104_169 (SEQ ID NO: 12)—was generated with the folding analysis (100% fold change & codons with frequency of at least 15%). It contains the original 104 changes introduced in FOLD_104, and an additional 65 changed originated from clusters of evolutionarily conserved strong folding found using p-value threshold of 0.07. The extra 5 clusters found (located not close to the previous ones, see FIG. 1) were added to the already changed codons of variant FOLD_104.

UR_99 (SEQ ID NO: 13)—this variant was generated using an algorithm that searches for sequence in the Zika coding sequence (CDS) that are under-represented relative to a randomized model. Specifically, oligos of 5 nucleotides (called 5-mers) were identified which were underrepresented in frame 1 or frame 2 of the ORF. Only the 5-mers that appeared in both of reading frames were used and were integrated into the coding sequence by the alteration to synonymous codons. Methods of attenuating viruses with underrepresented sequences are disclosed in International Patent Publication WO2021205462, herein incorporated by reference in its entirety.

KL_296 (SEQ ID NO: 14)—this variant is designed such that it will kill the virus. Specifically, the codons in conserved locations were replaced with the rarest codons in the multiple sequence alignment (MSA) of all Zika sequences. In cases where there is a codon with row frequency of 0% (i.e., this codon does not appear in this row), we take the rarest codon in the sequence. The mutations were relatively uniformly distributed across the sequence (FIG. 1).

### Example 2: Zika Virus (ZIKV) Synthetic Variants of the Structural Proteins

Variants were also designed for the sequence encoding the ZIKA structural proteins: protein C, protein E and protein prM. The DNA sequence encoding the structural proteins is located at positions 108-2519 in the Zika genome (SEQ ID NO: 15). Five additional variants that attenuate the virus were generated based on the previous approaches (with some improvements).

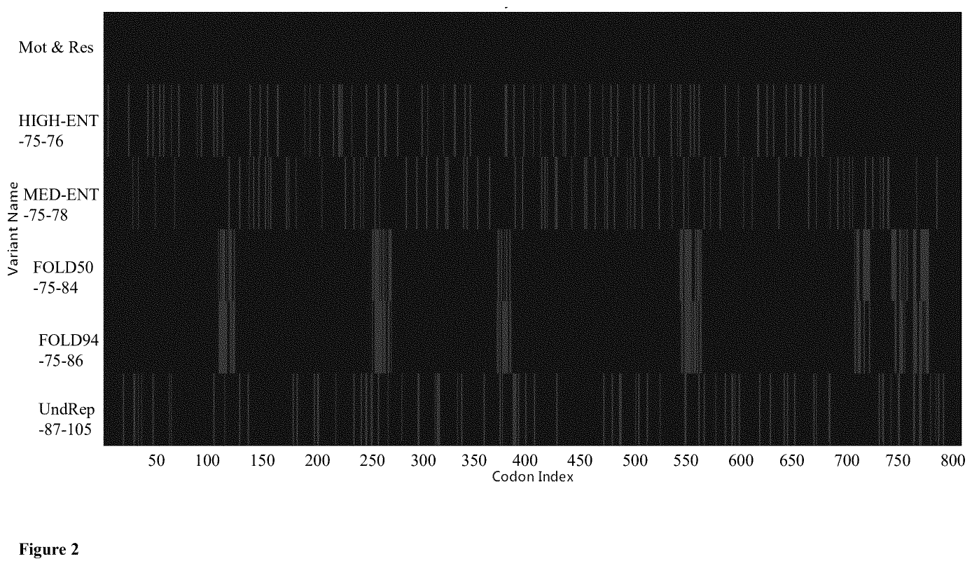

Entropy analysis/rare codon approach (2 variants): Of the 804 total codons within the structural proteins (SEQ ID NO: 16) 261 synonymous codons were identified that met the required criteria as described for the entropy approach hereinabove. If there were more than one possible candidate codon, the codon with the highest relative frequency (most evolutionarily conserved) was chosen. A variant with 75 synonymous replacements and high entropy change (ENT_HIGH_75_76, SEQ ID NO: 17) and one with 75 synonymous replacements but only medium entropy change (ENT_MED_75_78, SEQ ID NO: 18) (uniform distribution, mutually exclusive mutations, see FIG. 2) were generated.

Folding approach (2 variants): Variants deoptimized for local folding energy at positions that were determined to be functional were generated as before. Specifically, both the VCUB/Colum-Perm randomized models, found selection for 3 strong folding clusters and 4 weak folding clusters (FIG. 2). A variant with a 50% reduction in overall folding energy (Fold_75_84, SEQ ID NO: 19) is predicted to be less attenuated than a variant with 94% reduction (Fold_75_86, SEQ ID NO: 20).

Underrepresented sequences approach (1 variant): UR_87_105 (SEQ ID NO: 21) was generated by created underrepresented 5-mers as described hereinabove. The 5-mers chosen were the ones underrepresented in frame 1 and frame 2 of the ORF. The codons created were not rare ones with respect to the whole genome (i.e., their relative frequency was higher than 15% across all Zika genomes analyzed). Table 1 lists the underrepresented (UR) sequences of 5 nucleotides that were identified in ZIKA in each reading frame. Their corresponding p-values are also provided. Notably, the UR sequences differ between the three reading frames.

TABLE 1
Under-represented (UR) sequences in Zika.

| Frame 1 | | Frame 2 | | Frame 3 | |
|---|---|---|---|---|---|
| 5-mer | p-value | 5-mer | p-value | 5-mer | p-value |
| AGGGG | 2.0 · 10⁻⁶ | TCTTT | 1.5 · 10⁻⁴ | CGAGA | 7.0 · 10⁻⁶ |
| GATAT | 7.0 · 10⁻⁶ | GGGTG | 1.53 · 10⁻⁴ | GATCG | 3.6 · 10⁻⁵ |
| AAACA | 4.0 · 10⁻⁵ | CACTT | 1.68 · 10⁻⁴ | TATGT | 1.23 · 10⁻⁴ |
| CTCGA | 4.7 · 10⁻⁵ | GGACT | 2.24 · 10⁻⁴ | GTGGC | 1.95 · 10⁻⁴ |
| GGGCT | 5.5 · 10⁻⁵ | GGCTC | 5.8 · 10⁻⁴ | CAAAA | 3.08 · 10⁻⁴ |
| AGCCT | 8.4 · 10⁻⁴ | TGGTT | 7.77 · 10⁻⁴ | CAGGG | 3.4 · 10⁻⁴ |
| | | | | TATTG | 1.204 · 10⁻³ |

### Example 3: The In Vivo Effect of Zika Virus (ZIKV) Synthetic Variants in the AG129 Mouse Strain

Various strains of Zika virus (ZIKV) cause lethal infection and disease in the AG129 mouse strain. Depending on the strain, severe disease is observed around 1-2 weeks after virus challenge. It has been previously demonstrated that synthetic wild type (WT) ZIKV is lethal in the mouse model and has a similar disease phenotype as a WT isolate of ZIKV. Complete lethality, weight loss and various neurological disease signs are observed in mice infected with synthetic WT ZIKV.

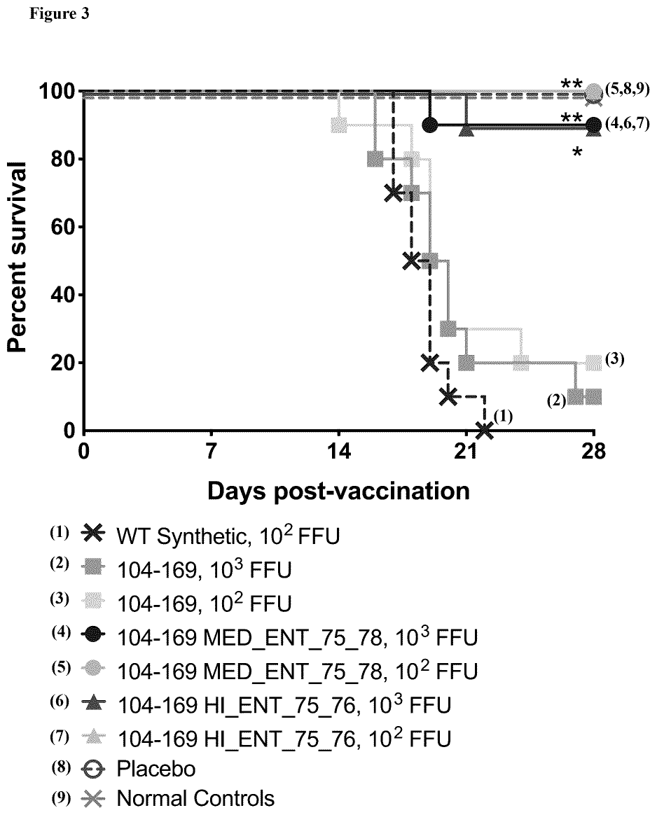

AG129 mice were vaccinated with a synthetic wild-type virus and lethality in all the mice was observed by 22 days post injection (FIG. 3, Table 2), which was similar to previous studies. Mice vaccinated with the FOLD_104-169 variant at a dose of either 10\hat{ }2 or 10\hat{ }3 focus-forming units (FFU) had a high mortality rate, but one that was generally delayed as compared with synthetic WT virus. Several of mice completely survived the challenge with the FOLD_104-169 variant. These vaccinated mice were then challenge with an Asian strain of ZIKV and also survived. This indicates that even the lower dose of the Zika variant was sufficient to vaccinate and fully protect the mice.

Due to the high mortality still observed with this variant, two new combined variants predicted to be more attenuated was constructed. The combined variants contained changes in both the NS5 coding region and the region encoding the structural proteins. FOLD_104-169 ENT_MED_75_78 and FOLD_104-169 ENT_HIGH_75_76 combined a folding variant in the NS5 region with an entropy variant in the structural protein region. Mice were vaccinated with both of these constructs at two concentrations and these variants were found to be far less lethal (FIG. 3). Indeed, no mortality was observed at the 10\hat{ }2 FFU dose and only 10% mortality was observed at the 10\hat{ }3 FFU dose. These improvements in survival are significant as compared with the synthetic wild-type virus and the NS5 only variant.

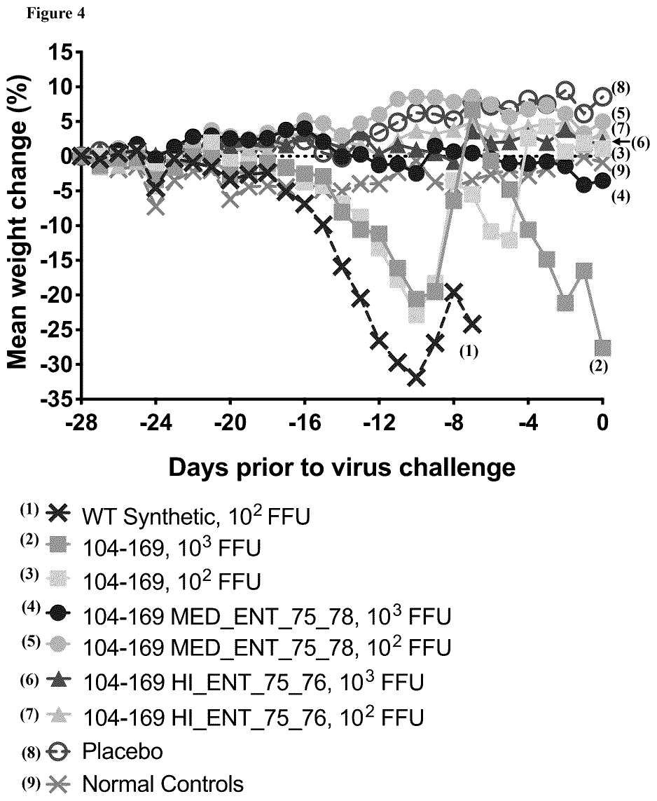

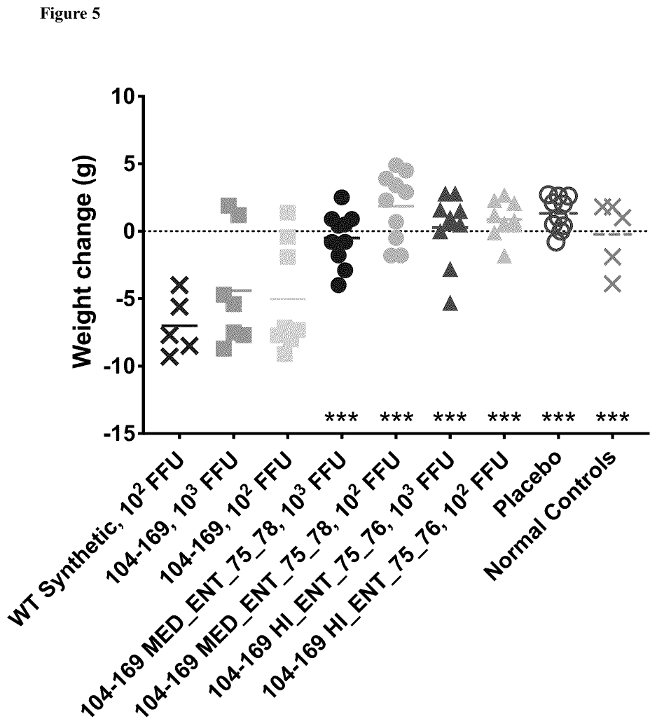

Weight change during the vaccination period mirrored survival curves, with animals vaccinated with WT or FOLD_104-169 losing weight during this time (FIG. 4). Weight curves appeared to rebound after day-13 dpi, which corresponded with later mortality or survival through the vaccination period (FIG. 4). Significantly improved average weight was observed in groups of mice vaccinated with the combined variants, which was similar to that of placebo-vaccinated and normal control groups (FIG. 5, Table 2).

TABLE 2
Summary results of AG129 vaccination with attenuated synthetic Zika virus.

| | | Duration of experiment: 57 days | | | | |
|---|---|---|---|---|---|---|
| | | Treatment vol./schedule: 0.1 mL, single IP injection −28 dpi | | | | |
| | | | | Pre- | Post- | |
| Animals: AG129 mice | | | | challenge | challenge | |
| Virus/route: WT ZIKV | | | | mean wt. | mean wt. | |
| Asian strain/s.c. | | Alive/ | MDD^{a} ± | change^{b} | change^{c} | |
| Treatment | Virus | total | SD | (g) ± SD | (g) ± SD | Viremia^{d} |
| WT Synthetic, | WT | 0/10 | −9.4 ± 1.6 | −7.0 ± 2.2*** | — | — |
| 10² FFU | ZIKV | | | | | |
| 104-169, | WT | 0/10 | −7.5 ± 4.3 | −4.4 ± 4.3*** | — | — |
| 10³ FFU | ZIKV | | | | | |
| 104-169, | WT | 2/10 | −3.5 ± 11.6 | −5.0 ± 4.0*** | 3.6 ± 0.4*** | −1.8 ± 0.2 |
| 10² FFU | ZIKV | | | | | |
| 104-169 | WT | 7/10 | 1.3 ± 10.0 | −0.5 ± 2.0 | 1.1 ± 0.9** | −1.1 ± 1.6* |
| MED_ENT_75_78, | ZIKV | | | | | |
| 10³ FFU | | | | | | |
| 104-169 | WT | 8/10 | 7.5 ± 4.9 | 1.9 ± 2.5 | 1.1 ± 1.3** | −0.9 ± 0.9* |
| MED_ENT_75_78, | ZIKV | | | | | |
| 10² FFU | | | | | | |
| 104-169 | WT | 9/10 | 6.5 ± 19.1 | 0.3 ± 2.5 | 0.2 ± 3.5** | −0.6 ± 1.0 |
| HI_ENT_75_76, | ZIKV | | | | | |
| 10³ FFU | | | | | | |
| 104-169 | WT | 8/10 | 10.5 ± 7.8 | 0.9 ± 1.3 | 0.3 ± 1.0** | −0.2 ± 1.3 |
| HI_ENT_75_76, | ZIKV | | | | | |
| 10² FFU | | | | | | |
| Placebo | WT | 2/10 | 16.4 ± 2.3 | 1.3 ± 1.3 | −4.6 ± 3.5 | 0.9 ± 2.1 |
| | ZIKV | | | | | |
| Normal | — | 5/5 | >28.0 ± 0.0 | −0.2 ± 2.6 | 1.6 ± 0.4** | −1.6 ± 0.5** |
| Controls | | | | | | |

^{a}Mean day of death. Values are average days pre- (negative values) or post-virus challenge.
^{b}Difference between weight on −28 and −10 days post-virus challenge representing maximal weight change within the vaccination period of this study.
^{c}Difference between weight on 0 and 17 days post-virus challenge representing maximal weight change within this study.
^{d}Serum was collected 3 dpi. Values are mean virus titer ± SD.
***P < 0.001, P < 0.01,
*P < 0.05 as compared to placebo treatment.

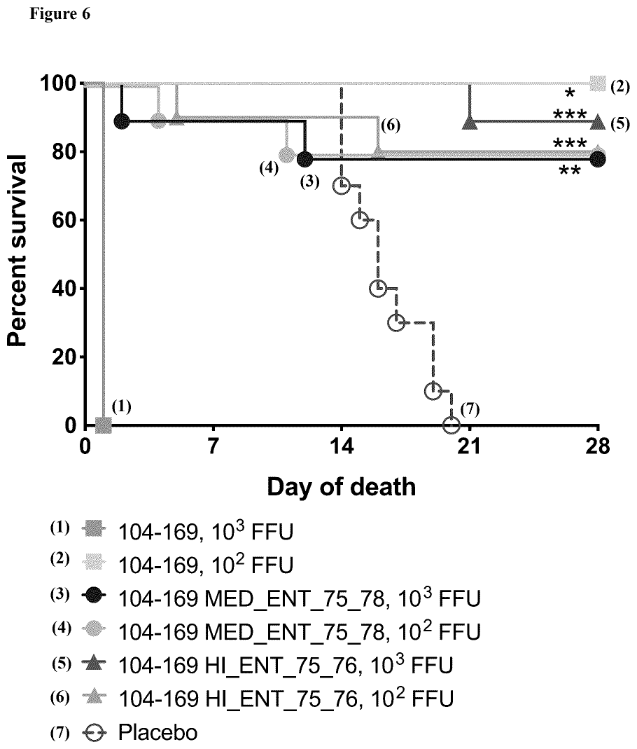

Vaccinated animals (those that survived the variant virus) were infected with a Malaysian strain of ZIKV to test protection associated with the vaccine. Serum is collected from all surviving mice just prior to virus challenge to evaluate the presence of neutralizing antibody. Neutralizing antibodies are found to be present in surviving mice. Vaccinated mice were significantly protected from mortality as compared with mice that were given the placebo (FIG. 6). Some early mortality occurred after virus challenge, but this was likely due to later stage mortality associated with vaccination, as mice infected with the Malaysian strain of ZIKV do not tend to succumb to disease prior to day 10 post infection. A minority of mice vaccinated with the combined variant succumbed to virus challenge, but 80-90% of mice were protected after vaccination (FIG. 6). Thus, the combined variant attenuated synthetic ZIKVs were generally well-tolerated and generated a protective response to a lethal challenge with WT ZIKV.

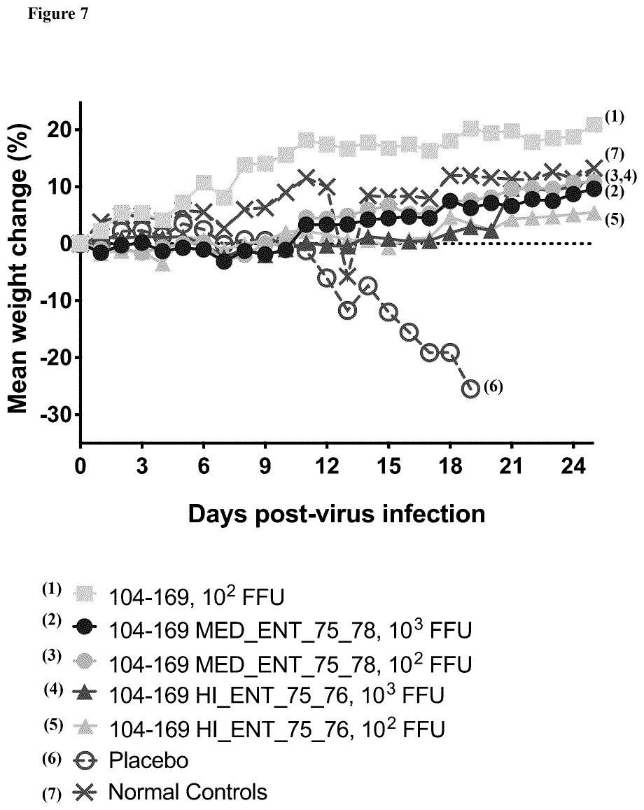

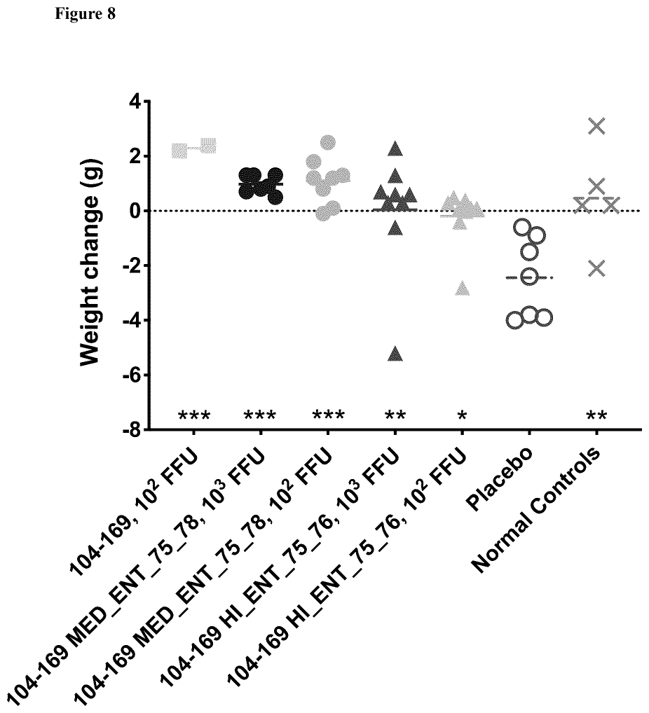

Vaccinated mice also tended on average to maintain or gain weight during the challenge portion of the study, as compared with placebo-injected mice that experienced an average weight loss beginning at day 11 post injection (FIG. 7). Indeed, significant improvement in average weight change between days 5 and 14 post injection was observed in all vaccinated mice, which was similar to normal controls (FIG. 8, Table 2).

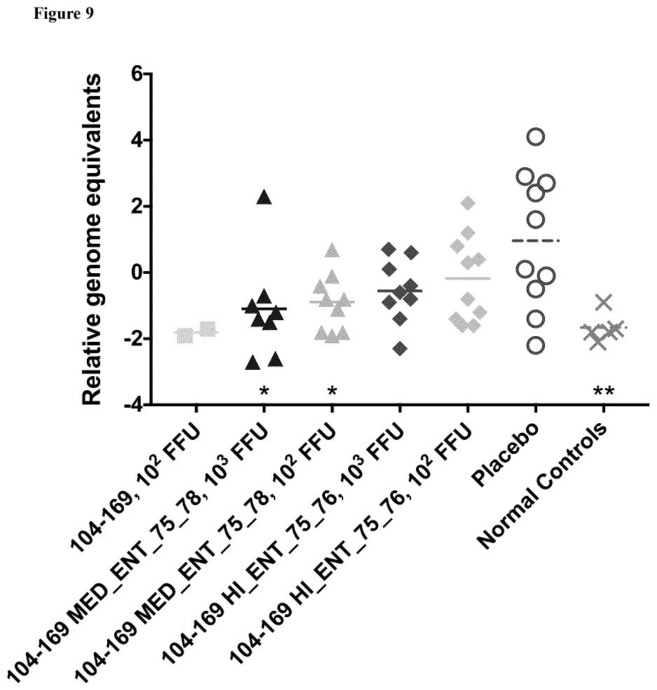

Total RNA was extracted from serum collected 3- and 7-days post infection (dpi) with the WT virus. Total ZIKV RNA was quantified by qRT-PCR. A relatively low signal was detected on day 3, which is consistent with this model. There was a significantly (P<0.05) lower amount of viral RNA observed in mice vaccinated with the FOLD_104_169_MED_ENT_75_78 combined synthetic ZIKV variant as compared with placebo (FIG. 9). There was also a trend towards reduction in the groups vaccinated with the other variants. Analysis of samples collected 7 dpi show significantly reduced virus in the vaccinated mice.

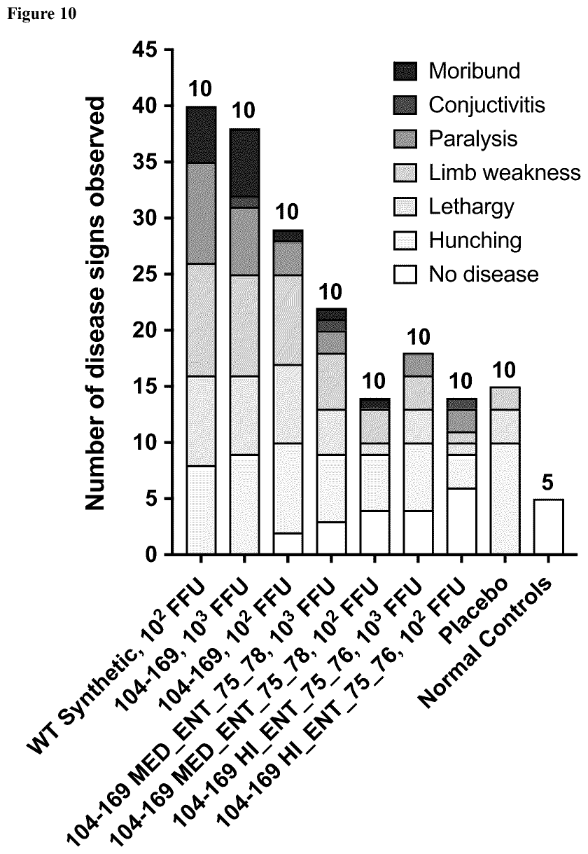

Disease signs (symptoms) were recorded for vaccinated mice during the vaccination period as well as after virus challenge. There was a general decrease in disease signs in mice vaccinated with the combined synthetic Zika viruses, although a few mice displayed some severe signs of disease (FIG. 10). These results correlate with the above disease parameters and further demonstrate the benefit of vaccination with these improved synthetic attenuated ZIKVs.

It is clear that the combined Zika variants were well-tolerated and produced a protective effect in AG129 mice when administered 28 days prior to virus challenge. Additionally, significant improvement in many disease parameters were observed after lethal challenge with WT ZIKV. These results clearly demonstrate the superiority of the current variants over other attenuated viruses known in the art.

### Example 4: Hepatitis C Virus (HCV) Synthetic Variants of the NS5 Protein Coding Region

The HCV genome encodes a polyprotein that includes all 10 HCV proteins. Variants of the region that encodes the NS5A and NS5B proteins, which are predicted to cause virus attenuation, were generated based on four approaches/measures (which are not mutually exclusive). The DNA sequence encoding the polyprotein containing NS5A/B is located at positions 6257-9427 in the HCV genome (SEQ ID NO: 22).

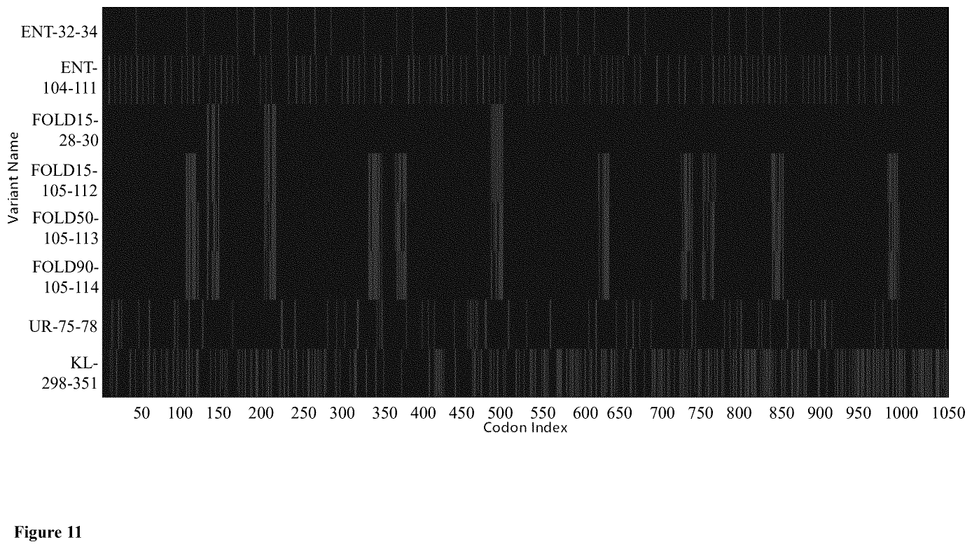

Entropy analysis/rare codon approach (2 variants): Of the 1057 total codons within NS5A/B (SEQ ID NO: 23) 720 synonymous codons were identified that met the required criteria described hereinabove (but with a 10% frequency threshold instead of 15%). If there were more than one possible candidate codon, the codon with the highest relative frequency (most evolutionarily conserved) was chosen. A variant with 32 synonymous replacements (ENT_32_34, SEQ ID NO: 24) and one with 104 synonymous replacements (ENT 104_111, SEQ ID NO: 25) (uniform distribution, mutually exclusive mutations, see FIG. 11) were generated.

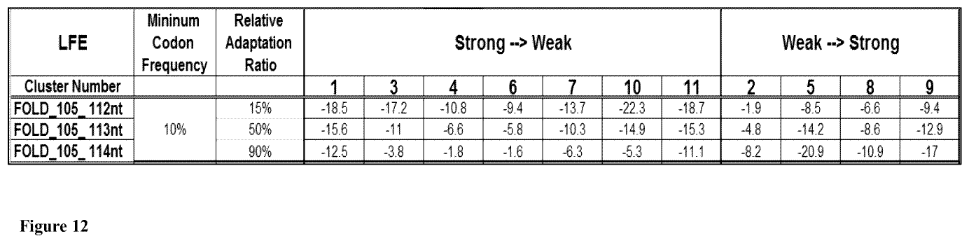

Folding approach (4 variants): Variants deoptimized for local folding energy at positions that were determined to be functional were generated as before (FIG. 11). 11 regions of conserved folding were found, 7 of which had strong folding and 3 of which had weak folding. The 11 regions can be seen in FIG. 11 and the changes produced by the variants are shown in FIG. 12. The variant with a 15% reduction (FOLD_105_112, SEQ ID NO: 27) is predicted to be less attenuated than the variant with 50% reduction (FOLD_105_113, SEQ ID NO: 28), which is itself predicted to be less attenuated than the 90% reduction (FOLD_105_114, SEQ ID NO: 29). An additional variant was generated also with 15% reduction but only over part of the coding region (FOLD_28_30, SEQ ID NO: 26). This variant was predicted to be the least attenuated. It is significant that the first three folding variants all had the same number of synonymous substitutions, but that these substitutions produced very different changes in folding energy. It is the change in folding energy that correlates with attenuation and not the number of substitutions.

Underrepresented sequences approach (1 variant): UR_75_78 (SEQ ID NO: 30) was generated by created underrepresented 5-mers as described hereinabove. The 5-mers chosen were the ones underrepresented in frame 1 and frame 2 of the ORF. The codons created were not rare ones with respect to the whole genome (i.e., their relative frequency was higher than 10% across all HCV genomes analyzed). Table 3 lists the underrepresented (UR) sequences of 5 nucleotides that were identified in HCV in each reading frame. Their corresponding p-values are also provided. Notably, the UR sequences differ between the three reading frames.

TABLE 3
Under-represented (UR) sequences in Hepatitis C.

| Frame 1 | | Frame 2 | | Frame 3 | |
|---|---|---|---|---|---|
| 5-mer | p-value | 5-mer | p-value | 5-mer | p-value |
| AACGA | 8.0 · 10⁻⁶ | CGATC | 8.0 · 10⁻⁶ | GATGG | 1.5 · 10⁻⁶ |
| TTCGA | 1.1 · 10⁻⁴ | GTGGG | 2.5 · 10⁻⁵ | CCCAG | 1.4 · 10⁻⁵ |
| ACCCC | 1.35 · 10⁻⁴ | TCTGG | 4.0 · 10⁻⁵ | GGTTG | 2.0 · 10⁻⁵ |
| AACTG | 2.34 · 10⁻⁴ | AGGAT | 1.19 · 10⁻⁴ | CGAGT | 9.9 · 10⁻⁵ |
| GACAC | 4.32 · 10⁻⁴ | CCCCA | 1.36 · 10⁻⁴ | CGACA | 4.4 · 10⁻⁴ |
| GAAGG | 4.64 · 10⁻⁴ | CCTCC | 2.7 · 10⁻⁴ | GGATC | 6.4 · 10⁻⁴ |
| GTCTG | 2.0 · 10⁻⁴ | ACGAC | 3.12 · 10⁻⁴ | GATGC | 8.96 · 10⁻⁴ |
| CGGCT | 5.1 · 10⁻⁴ | TGGTT | 5.78 · 10⁻⁴ | | |
| TGCAG | 9.57 · 10⁻⁴ | TCCTC | 5.85 · 10⁻⁴ | | |
| TCGAT | 1.1 · 10⁻³ | CTTAC | 7.02 · 10⁻⁴ | | |
| GGCTA | 1.1 · 10⁻³ | CGACA | 1.4 · 10⁻³ | | |
| AAAGG | 1.3 · 10⁻³ | AAGGG | 2.3 · 10⁻³ | | |
| | | CGATG | 2.3 · 10⁻³ | | |

A variant that will kill he virus (KL_298_351, SEQ ID NO: 31) was also generated as described hereinabove. Once again, the mutations were relatively uniformly distributed across the sequence (FIG. 11).

### Example 5: HCV Synthetic Variants Attenuate Viral Replication and Pathogenesis (Overview)

Viral names: It should be noted that variant FOLD_28_30 (SEQ ID NO: 26) is referred to in the following section as HCVmut3 and “Strong” based on its viral fitness. Variant FOLD_105_112 (SEQ ID NO: 27) is referred to as HCVmut4 and “Mid”. Variant UR_75_78 (SEQ ID NO: 30) is referred to as HCVmut7 and “Weak”. Variant (FOLD_105_113, SEQ ID NO: 28) is referred to as HCVmut5. Variant FOLD_105_114 (SEQ ID NO: 29) is referred to as HCVmut6. Variant ENT_32_34 (SEQ ID NO: 24) is referred to as HCVmut1. Variant ENT_104_111 (SEQ ID NO: 25) is referred to as HCVmut2. Variant KL_298_351 (SEQ ID NO: 31) is referred to as HCVmut8.

Flow cytometry and cell sorting: Huh7.5 cells were infected with HCV mutant viruses at MOI of 0.1 and passaged for two weeks until achieving approximately 100% infection in WT. Then, cells were collected and washed with PBS at least twice. Cells were fixed with 4% PFA (SigmaAldrich) for 10 min and permeabilized with 0.1% saponin (SigmaAldrich) in molecular grade PBS (Biological Industries) for 10 min. Cells were pelleted by centrifugation at 3000 rpm for 7 min and washed with Wash Buffer: PBS containing 0.5% BSA (MP Biomedicals), 0.01% saponin (Sigma-Aldrich). Then, the cells were incubated with primary antibodies (anti-HCV core antibody invitrogen) with 5% goat serum to block unspecific antibody binding at 37° C. for 2h, followed with fluorochrome-labelled antibody (Alexa Fluor® 488-AffiniPure Donkey Anti-Mouse IgG) for 1 h in the dark.

Following secondary antibody staining, cells were washed twice in wash buffer and resuspended with PBS. Cells were sorted on the BD FACSAria™ III (BD Biosciences) using FACSDiva software. Gates were set with reference to negative controls. The sorting speed was adjusted to ensure sorting efficiency above 90%. Cells were collected in tubes that were coated with a small amount of Sort buffer.

After sorting, cells were pelleted by centrifugation at 3000 rpm for 5 min. The supernatant was discarded and total RNA was isolated from the pellet as described below.

RNA isolation and quantitative reverse transcription-PCR analysis: Total RNA from fresh HCV-infected and a non-infected Huh7.5 cell was purified using the RNeasy Mini Kit (Qiagen). Following sort, total RNA was isolated from the pellet using the MasterPure™ Complete DNA and RNA Purification Kit, Lucigen. Equal amounts of total RNA were reverse transcribed using the high-capacity cDNA reverse transcription kit (Applied Biosystems, Foster City, California). Quantitative RT-PCR was performed using the StepOne RT-PCR system. Briefly, 2 μg of total RNA were used in 20 μl reverse transcription assays. Subsequently, 2 μl (10 ng) of the reverse transcription product were used as a template for qPCR in a 10 μl volume of PCR reaction (SYBR Green PCR Mix, Applied Biosystems, Foster City, CA, USA) using specific forward and reverse primers (Table 1).

Differential expression was calculated using the equation of 2 (−ΔΔCt), with the GAPDH as the endogenous controls. RT-PCR analysis was conducted using RNA from three independent experiments.

RNA-Seq: Huh7.5 cells were infected with WT and HCVmut7 viruses. Total RNA from HCV-infected and non-infected cells was purified using the RNeasy Mini Kit (Qiagen). RT was performed using SuperScript IV Reverse Transcriptase (Invitrogen). The integrity of the isolated RNA was tested using the Agilent High Sensitivity RNA Kit and Tapestation 4200. 500 ng of total RNA were used for libraries preparation using the Quantseq3′ mRNA-Seq prep kit FWD (Lexogen) according to manufacturer's instructions. Quantification of the library was performed using dsDNA HS Assay Kit and Qubit 2.0 (Molecular Probes, Life Technologies) and qualification was done using the Agilent D1000 Tapestation Kit and Tapestation 4200. 20 nM of each library was pooled together and was diluted to 4 nM according to NextSeq manufacturer's instructions. 1.45 μM was loaded onto the Flow Cell with 1% PhiX library control. Libraries were sequenced on an Illumina NextSeq 550 instrument, 75 cycles single read sequencing.

RNA-seq mapping and quantification and variant calling: RNA-seq reads were aligned to the GRCh38 transcriptome, downloaded from NCBI. The reads were aligned, counted, and normalized using salmon 1.2.1 (mapping-based mode with default parameters).

Differential expression and functional enrichment analysis: Differential expression (DE) analysis was conducted using DESeq2, assessing differentially expressed genes (DEGs) between uninfected host cell (control) and infected host cells (WT/variants); and, separately, DEGs between WT-infected host cells and variant-infected host cells.

The inventors used g: Profiler's built-in Gene Ontology (GO) term database to analyze their enrichment in the DEGs found by DESeq2. The inventors also downloaded and similarly analyzed pathways from MSigDB, set C2, using g: Profiler for this data as well.

Deep sequencing of NS5A/NS5B amplicons: Huh7.5 cells were infected with WT and HCVmut7 viruses. Following two weeks of infection, total RNA from infected cells was purified using the RNeasy Mini Kit (Qiagen). RT was performed using SuperScript IV Reverse Transcriptase (Invitrogen). Specific primers to the NS5A/NS5B genes of HCV were designed for four overlapping amplicons (Table 2). PCR amplification of the cDNA and plasmids of HCVmut7 and WT HCV (as controls) was performed using Phusion® High-Fidelity DNA Polymerase (M0530): initial denaturation for 30 sec at 98° C., followed by 35 cycles of denaturation for 10 sec at 98° C., annealing for 30 sec at 60° C. extension for 30 sec at 72° C., and final extension for 2 min at 72° C. The NS5A/NS5B amplicons were gel purified and the concentration was determined by Qubit. 25 ng of the DNA purified product were used for libraries preparation using the NEBNext Ultra II FS DNA kit (NEB, #E7805) according to the manufacturer's instructions. Quantification of the library was performed using dsDNA HS Assay Kit and Qubit 2.0 (Molecular Probes, Life Technologies) and qualification was done using the Agilent D1000 Tapestation Kit and Tapestation 4200. 5 nM of each library was pooled together and was diluted to 4 nM according to NextSeq manufacturer's instructions. 1.6 μM was loaded onto the Flow Cell with 1% PhiX library control. Libraries were sequenced on an Illumina NextSeq 550 instrument, 125 cycles paired end sequencing.

Variant calling in NS5A/NS5B amplicons: HCV deep sequencing reads were mapped to the 4 viral fragment sequences using BWA-MEM. Duplicate reads were marked using GATK MarkDuplicates. SAM/BAM file sorting and indexing was done using samtools. We used two different tools for variant calling: freebayes, with the parameter −F 0.05; and LoFreq, with the parameter—call-indels. All other parameters were set to the default values. We filtered out mutations whose frequency was smaller than 5%, and indels that appeared in homopolymer runs (HRun) of at least 5 nucleotides. Only mutations that were identified by both tools were kept.

Cell migration and invasion assay: Transwell assay was performed as is known in the art. In brief, 1×10\hat{ }5 infected and non-infected Huh7.5 cells were seeded in the upper compartment of Transwell inserts (Corning) coated and uncoated with Matrigel (Corning).

The upper compartment contained DMEM with 0.5% FBS and the lower compartment contained DMEM with 10% FBS. Cells were allowed to invade for 24 hours. At the end of the incubation, cells that migrated through the membrane of the insert were stained using commercial staining (Bioconsult). Transwell inserts with cells were visualized using Zeiss apotome system with ×5 DIC lens.

Matrix degradation assay: Matrix degradation assay was performed as is known in the art. In brief, infected and non-infected Huh7.5 cells were cultured on fluorescently labelled Gelatine matrix for 72 hours before fixation. Degradation was analyzed by quantifying the average degraded area pixels per field using ImageJ software and normalized to the number of cells in each field.

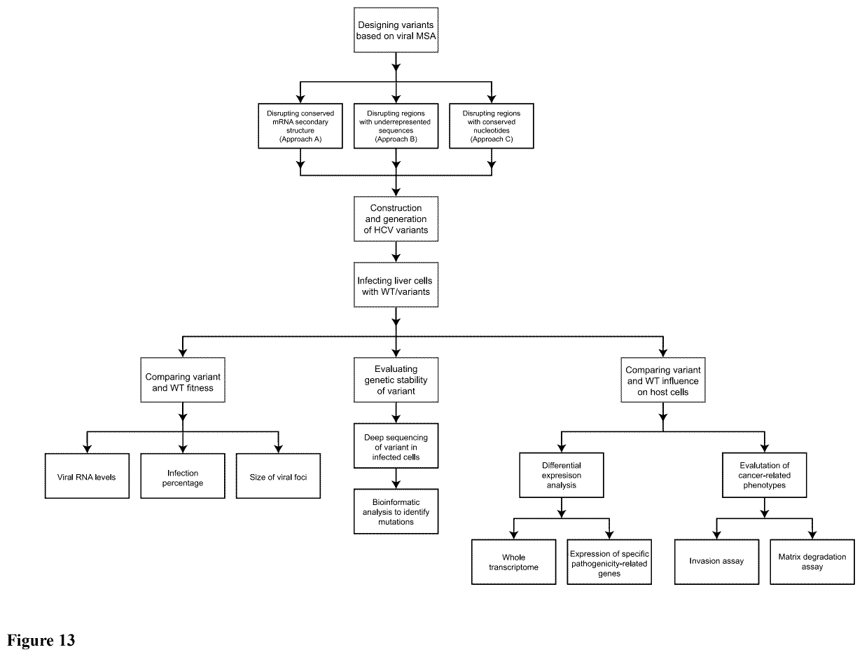

The overall approach performed hereinbelow is summarized in FIG. 13 and includes performing MSA of HCV sequences for designing attenuated HCV variants, based on three approaches (disrupting mRNA folding, underrepresented sequences or conserved positions); production of the HCV variants; evaluating their viral fitness by measuring viral RNA replication and viral spread in liver cells; validating their genetic stability; and investigating their pathogenicity by evaluating differentially expressed genes (DEGs) and cancer-related phenotypes.

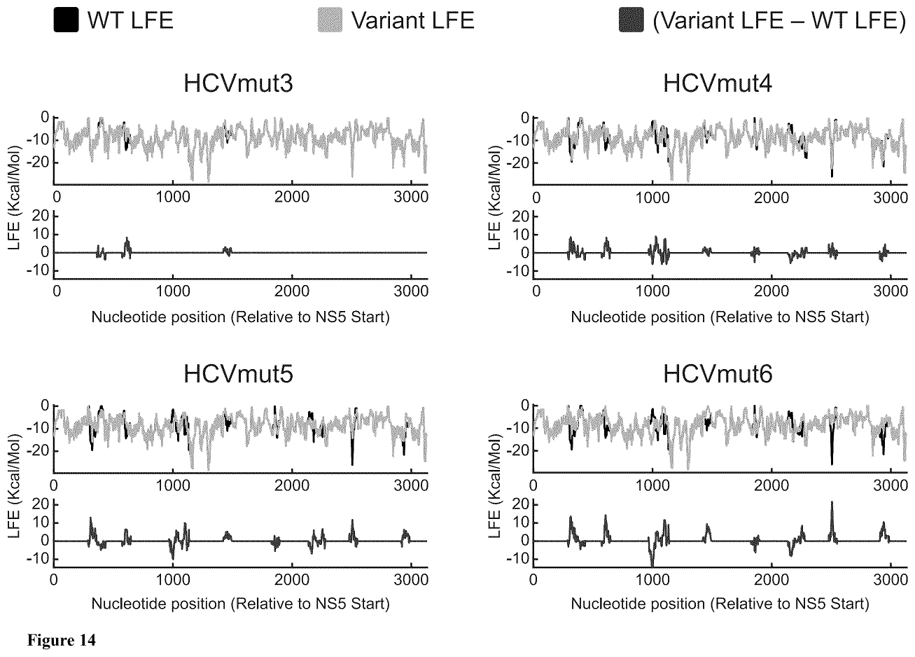

mRNA secondary structure is a major determinant of translation and organismal fitness. It was reasoned that disrupting the mRNA structure in regions with selection for strong (or weak) folding would reduce the viral fitness; therefore, MSA information was used to identify regions with a significantly strong/weak folding and synonymous mutations that are predicted to change the folding in these regions to be weak/strong (respectively) were engineered (FIG. 14).

MSA information was also used to find regions with underrepresented (UR) 5-mers, which are assumed to consist of adverse regulatory elements (e.g. nucleotide-level sequences that serve as signals for RNA-binding factors). Mutations in these regions were designed, changing the codons to deliberately insert these UR sequences, which may disrupt the gene's function.

Finally, variants were designed based on Shannon entropy, a measure of information uniformity and uncertainty originating in information theory. Shannon entropy was used to assess the evolutionary conservation of each genomic position, based on the MSA generated; positions with medium-to-high conservation were then mutated to synonymous codons.

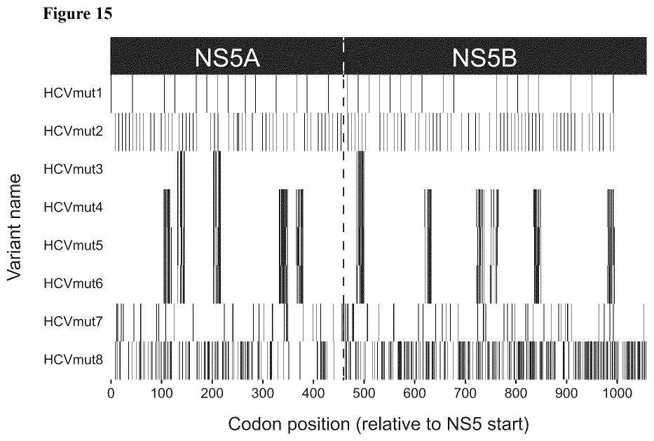

Overall, eight HCV variants containing synonymous mutations in HCV NS5A/NS5B sequences were designed (FIG. 15). Among these, three variants (HCVmut1-2, 8) were based on sequence conservation; four variants (HCVmut3-6) were generated based on RNA folding analysis; and one variant (HCVmut7) was generated by considering UR sequences.

The sequences of the eight HCV variants were synthesized, cloned into the pHJ3-5 vector and transcribed into RNA. Each synthetic virus' resulting RNA was separately electroporated into FT3-7 cells, infectious mutant viruses (HCVmut1-8) were generated, and virus stocks were prepared for each variant. It was found that the RNA of mutant viruses HCVmut2 and HCVmut8, which contained the highest number of mutations, did not produce replicating genomes. Therefore, these variants were excluded from the study. Table 5 summarizes the data for each variant, including the strategy of attenuation, the number of changed nucleotides and codons, and additional details regarding the level of attenuation.

TABLE 5
The virus variants designed for the study

| | | Number of | Number of | |
|---|---|---|---|---|
| | Variant name | changed | changed | |
| Category | (SEQ ID NO:) | nucleotides | codons | Note |
| Sequence | HCVmut1 (24) | 34 | 32 | — |
| conservation | HCVmut2 (25) | 111 | 104 | Did not produce |
| (Approach C) | | | | replicating |
| | | | | genomes, excluded |
| | | | | from further study. |
| Thermodynamics | HCVmut3 (26) | 30 | 28 | Used as the |
| | | | | “Strong” variant. |
| | HCVmut4 (27) | 112 | 105 | Used as the “Mid” |
| | | | | variant. |
| | HCVmut5 (28) | 113 | 105 | — |
| | HCVmut6 (29) | 114 | 105 | — |
| Underrepresented | HCVmut7 (30) | 78 | 75 | Used as the |
| sequences | | | | “Weak” variant. |
| Sequence | HCVmut8 (31) | 315 | 298 | Did not produce |
| conservation | | | | replicating |
| | | | | genomes, excluded |
| | | | | from further study. |

### Example 6: Engineered Variants have a Lower Viral Fitness Relative to the WT Virus

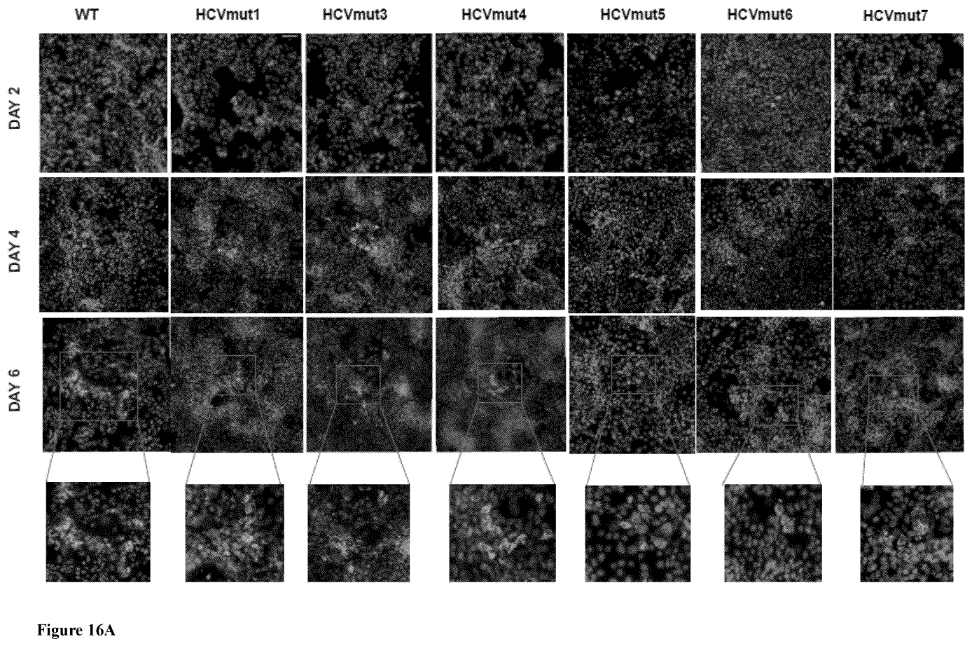

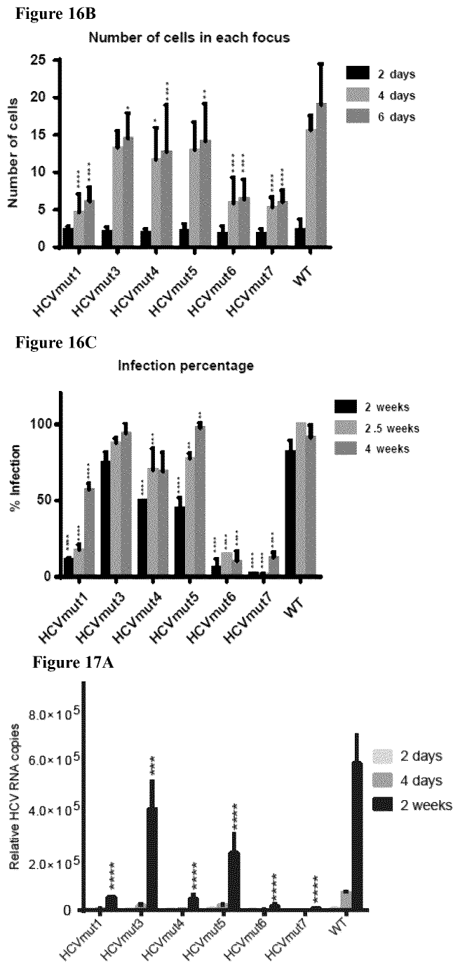

To assess the effect of the synonymous mutations on viral fitness, the inventors first evaluated the ability of the HCV variants to infect and spread. For comparing virus fitness, stocks of the WT and mutant viruses were tittered. Then, Huh7.5 cells were infected with 100 FFUs of mutant and WT viruses, and the number of infected cells comprising each focus were counted at 2, 4 and 6 days post infection by immunofluorescence that detects the viral proteins. Smaller foci, and therefore reduced cell-to-cell spread, were observed over time in the variants compared to the WT virus; mutants HCVmut6 and HCVmut7 showed the lowest ability to spread (FIG. 16A-16B).

The inventors then measured the level of infection by infecting Huh7.5 cells with the HCV variants at MOI 0.1 and monitoring the percentage of infected cells at 2, 2.5 and 4 weeks post infection. It was observed that the WT virus reached 100% infection two weeks following the infection, while HCVmut3 and HCVmut5 reached this point 4 weeks following the infection. In infections with the other mutants, a high infection level was not observed, particularly with HCVmut6 and HCVmut7 where infection percentage during the 4 weeks was not significantly increased (FIG. 16C).

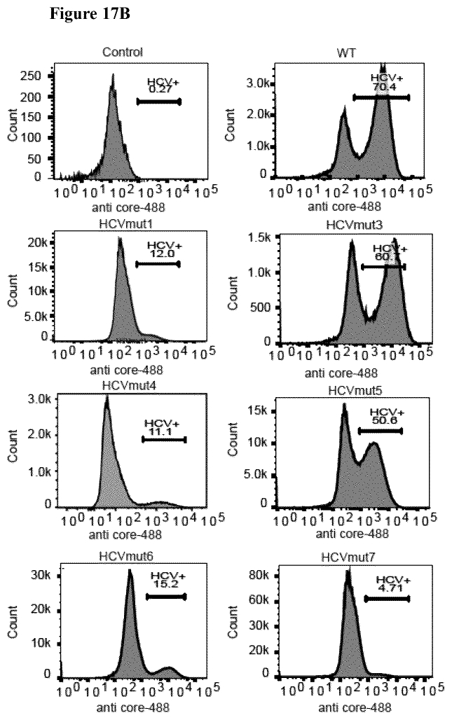

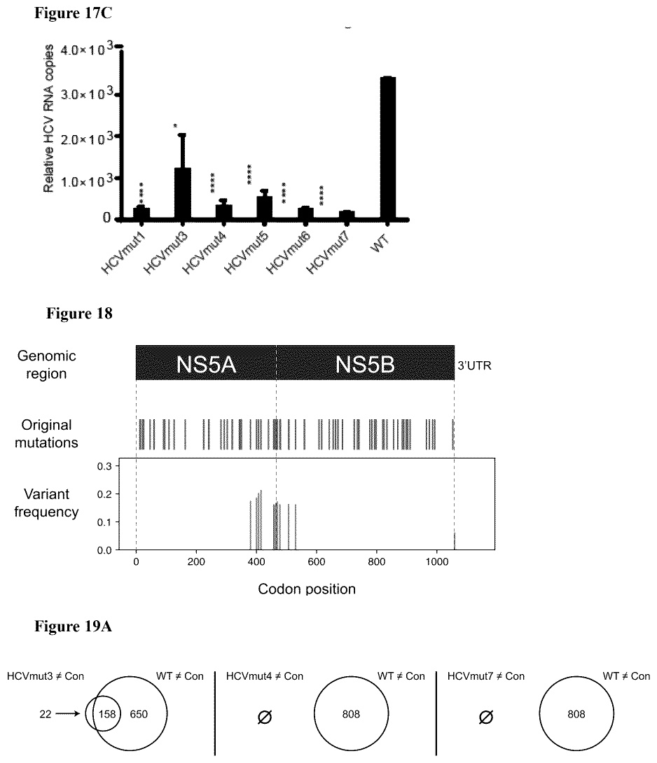

To further evaluate the effect of the synonymous mutations on viral fitness, the levels of HCV replication were measured by infecting Huh7.5 cells with the HCV variants and WT and performing qPCR for HCV RNA using 5′ primers at 2 days, 4 days and 2 weeks post infection. There was observed an overall reduction of HCV replication in the mutant viruses compared to the WT (FIG. 17A). The levels of replication of the different mutants were in accordance with the levels of infection and viral spread (FIG. 16A-16C), with HCVmut6 and HCVmut7 as the most attenuated variants.

To eliminate the possibility that the effect of reduced HCV RNA level observed is due to low percentage of infected cells rather than reduced fitness, the infected cells were isolated by FACS and HCV RNA levels were measured only in infected cells. Two weeks after infection, the cells were immunostained with anti-core to detect infected cells and sorted by FACS. Correspondence between the percent of infection observed by immunofluorescence and the percentage of infection observed by FACS was observed (FIG. 17B). Furthermore, an overall reduction of HCV RNA was observed in the mutant viruses as compared to the WT in sorted cells. The RNA levels of the different mutants were consistent with pre-sort HCV RNA (FIG. 17C).

From this point forward, the inventors focused on 3 variants: HCVmut3, HCVmut4 and HCVmut7, which were termed “Strong”, “Mid” and “Weak”, respectively. This is because in all 3 metrics-infection, spread and RNA level—the viral fitness ranking was WT>HCVmut3 (“Strong”)>HCVmut4 (“Mid”)>HCVmut7 (“Weak”).

### Example 7: Attenuated Variant HCVmut7 is Genetically Stable

The high number of inserted mutations in each variant is expected to constrain their reversion to WT. Indeed, low levels of viremia are observed in HCVmut7-infected cells at two and four weeks post infection, which do not rise above 15% of infected cells. To validate the stability of the attenuation on the genomic level, the inventors sequenced the genome of HCV at a region flanking the NS5A/NS5B genes, where mutations were inserted, after two weeks of infection with WT and HCVmut7 viruses in cell culture. Four overlapping amplicons were amplified and sequenced for each virus. For each virus there were obtained 2 repeats of 32-53M reads, with mapping rates of >90%. The average sequencing depth was ˜7890 mapped reads per position.

Variant calling was conducted using two programs: freebayes and LoFreq; and only mutations called by both programs were kept. 12 nucleotide changes in the HCVmut7 coding sequence compared to WT were identified; all 12 changes occurred in positions where mutations were inserted, within the overall 78 inserted mutations (FIG. 18, Table 6). All 12 changes were synonymous and reverted to the WT sequence. Among these, 7 resided within the NS5A sequence and 5 were within the NS5B sequence. The frequency of these changes ranged between 15-21% of all detected virus sequences. In addition, one mutation was detected at a low level, both for the WT and HCVmut7 virus, in the 3′UTR region, which is not among the inserted mutations. These findings, combined with the observed low level of viral fitness, which is stable over time, provide evidence for the genetic stability of the HCVmut7 variant.

TABLE 6
Variant calling results. Variants found in WT and HCVmut7 2 weeks post
infection. See “Attenuated variant HCVmut7 is genetically stable” in
the main text for details.

| | | | Genomic | Mutation | | Revert to |
|---|---|---|---|---|---|---|
| Position | Virus | Frequency | region | type | Mutation | WT? |
| 7396 | Mut7 | 17.3% | NS5A | SNP | A->C | TRUE |
| 7456 | Mut7 | 18.5% | NS5A | SNP | C->G | TRUE |
| 7477 | Mut7 | 20.1% | NS5A | SNP | A->C | TRUE |
| 7501 | Mut7 | 21.2% | NS5A | SNP | C->T | TRUE |
| 7625 | Mut7 | 16.1% | NS5A | SNP | AG->TC | TRUE |
| 7636 | Mut7 | 15.5% | NS5A | SNP | TGAC->CGAT | TRUE |
| 7648 | Mut7 | 16.5% | NS5A | SNP | C->G | TRUE |
| 7657 | Mut7 | 16.5% | NS5B | SNP | G->C | TRUE |
| 7663 | Mut7 | 17.0% | NS5B | SNP | T->A | TRUE |
| 7690 | Mut7 | 16.1% | NS5B | SNP | CCCATGC-> | TRUE |
| | | | | | TCCCTGT | |
| 7777 | Mut7 | 16.2% | NS5B | SNP | CTCC->ATCA | TRUE |
| 7846 | Mut7 | 16.1% | NS5B | SNP | C->T | TRUE |
| 9431 | Mut7 | 5.9% | 3'UTR | SNP | A->G | — |
| 9431 | WT | 7.3% | 3'UTR | SNP | A->G | — |

### Example 8: Engineered Variants Cause a Weaker Perturbation of Host Gene Expression Relative to the WT Virus

Accumulating evidence supports the involvement of epigenetic factors in promoting carcinogenesis in viral hepatitis-related HCC. It was previously demonstrated that HCV infection induces epigenetic alterations in hepatocytes that reprogram their gene expression profile. These epigenetic alterations are persistent as epigenetic signatures following virus eradication with DAAs in a “hit and run” mechanism. This HCV-induced epigenetic and gene expression perturbation is associated with oncogenic and invasive pathways and phenotypes in the host cell. Therefore, it is essential to evaluate the pathogenicity of the different HCV variants and examine their ability to alter host gene expression.

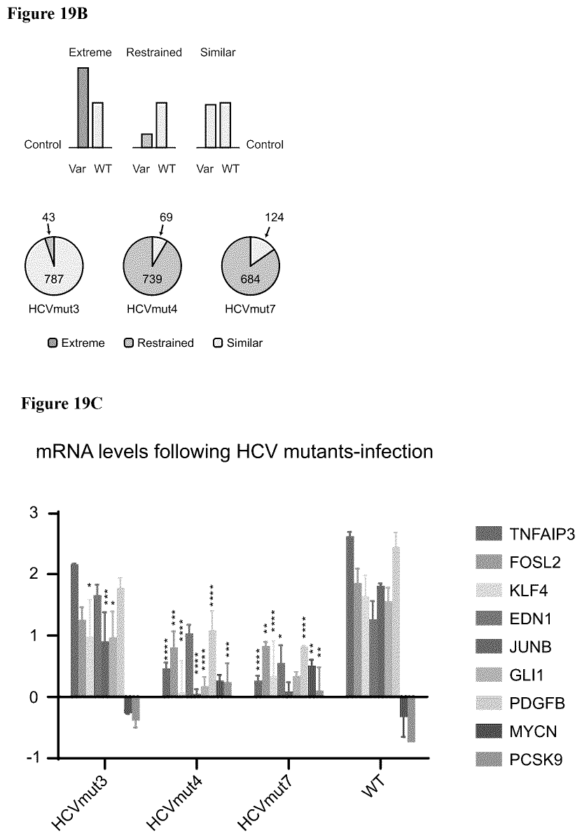

RNA-seq was performed for cells infected with mutant viruses at 2 weeks post infection and DE analysis was conducted on the host cell genes, comparing gene expression in the non-infected negative control cells to the expression in cells infected with each of the four viruses: WT, HCVmut3, HCVmut4 and HCVmut7. Gene expression under condition X was designated to be EX, where X can be either Control, WT, Strong (HCVmut3), Mid (HCVmut4) or Weak (HCVmut7). No DEGs were observed between EControl and EMid/EWeak, and 170 DEGs were observed under EStrong—a 5-fold decrease over 808 DEGs observed under EWT. An overlap of 158 genes between EWT and EStrong was observed (FIG. 19A).

Next, EWT was directly compared with each of the variants. The inventors focused on genes that were DE between EControl and EWT, and/or between EControl and EVariant (since it was assumed a gene that was expressed similarly under all 5 conditions is not significantly affected by the virus). All such genes were separated into 3 distinct groups (FIG. 19B, upper row): (A) Restrained expression: the variant virus caused a significantly smaller perturbation than the WT, i.e. EControl≤EVariant<EWT; (B) Extreme expression: the variant virus caused a significantly larger perturbation than the WT, i.e. EControl≤EWT<EVariant; and (C) Similar expression: the variant virus caused a perturbation comparable to the WT, i.e. EWT≈EVariant. Under EMid/EWeak, the expression of most genes was restrained, with some showing no DE between EVariant and EWT. Under EStrong, the perturbation in most genes was similar to EWT, with some genes being restrained (FIG. 19B, lower row). Overall, the perturbation in the host cell genes was significantly smaller under the variant viruses (especially “Weak” and “Mid”) relative to EWT—both when considering the number of perturbed genes and the magnitude of their perturbation.

Furthermore, the RNA-seq data was validate for specific genes that were previously identified as associated with an epigenetic signature following HCV infection and were correlated with HCC development. The ability of the HCV variants to induce alterations in expression of these genes was examined. Huh7.5-HS cells were infected with HCV mutant viruses and gene expression was evaluated two weeks post infection by qRT-PCR compared to control non-infected cells. The results demonstrate significant alterations in expression of these genes under EWT. Significant changes were observed under EStrong as well, but these changes were lower than those under EWT. Under EMid/EWeak minimal effect on host gene expression relative to the negative control were observed (FIG. 19C). Overall, the change in these genes was lower, relative to the change caused by the WT virus, under all 3 variants. This supports the findings regarding the lower viral fitness and pathogenicity of the variants.

### Example 9: HCV Variants have a Minimal Effect on Perturbation of Pathogenic Cellular Pathways Compared to WT

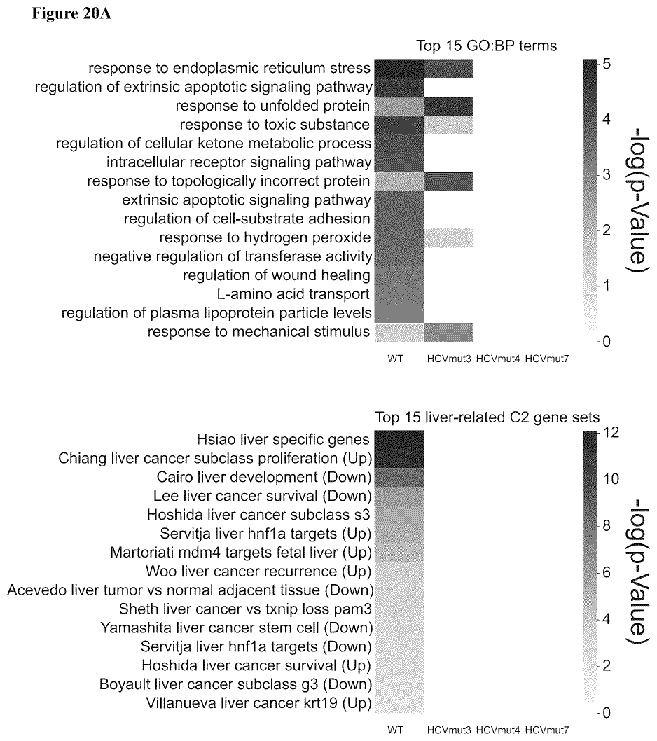

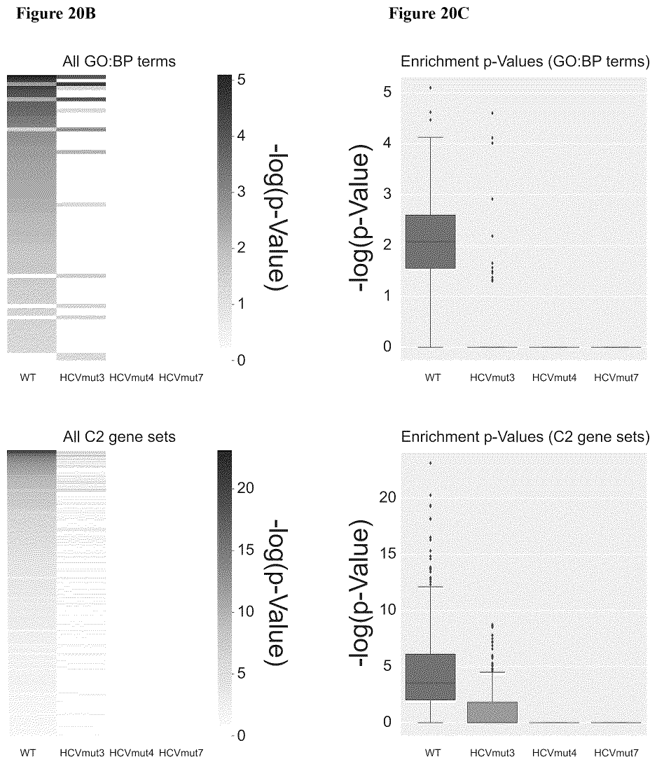

To further explore the nature of variants' attenuation, the inventors conducted functional enrichment analysis for GO pathway terms (GO:BP) and for the C2 gene sets, which are curated gene sets and pathways downloaded from the MSigDB database (FIG. 20A-20C).

The WT DEGs were enriched for substantially more GO terms and pathways, relative to the 3 variants: The WT virus perturbed genes that were significantly enriched in 71 GO pathways, with an average p-value of 0.01; comparatively, the Strong variant's DEGs were enriched in 13 GO terms, with an average p-value of 0.02. Since the Mid and Weak variants caused no significant DE, their change in expression wasn't significantly associated with any GO terms or pathways. The Strong variants' enriched GO terms were related to endoplasmic reticulum (ER) stress, incorrect folding of proteins, toxic response, and production of interferon beta. For the C2 gene sets, the WT virus' DEGs were significantly enriched in 440 sets, with an average p-value of 0.006; and the Strong variant's DEGs were enriched in 147 sets with an average p-value of 0.009. This is in accordance with the low number of DEGs caused by the Strong variant. The Strong variant's enriched C2 liver-related terms (i.e. terms which included the word “liver” in their name) were related to apoptosis, regulation by methylation, and hypoxia. These results further establish the significantly decreased effect of the variants on cellular functionality and induction of liver cancer related pathways and genes, relative to the WT virus.

### Example 10: Attenuated Variant HCVmut7 does not Affect Cellular Invasion Phenotype

The pathway analysis presented in FIG. 20A-20C reveals that the most significant pathways altered by HCV infection are related to cell motility and invasion. These pathways play a significant role in cancer cells which can degrade the extracellular matrix (ECM), invade, and migrate through the basement membrane that surrounds tumor cells and underline blood vessels. Indeed, in previous studies it was showed that HCV infection increases invasive abilities of host cells. Therefore, to evaluate whether the HCV attenuation affects cancer-related phenotypes, an invasion/migration assay and a matrix degradation assay were performed.

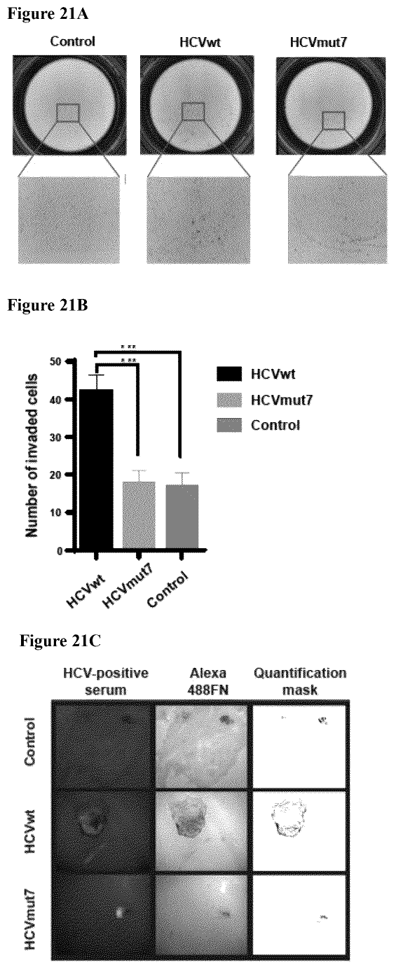

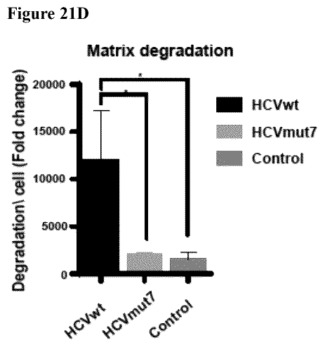

Huh7.5 cells were infected with WT or HCVmut7. For the invasion/migration experiment the cells were seeded on matrigel-coated transwells, and after 24 hours they were fixated and the number of cells that invaded through the Matrigel were counted. The results show higher invasive ability on WT infected cells compared to HCVmut7-infected cells and control non-infected cells (FIGS. 21A-21B). For the matrix degradation assay, the cells were seeded on labelled gelatin followed by fixation after 72 hours, and then the average degradation was calculated per field and normalized to the number of cells in each field. The results show a significant reduction in the degradation area following HCVmut7 infection, compared to WT-infected cells, with levels comparable to control non-infected cells (FIGS. 21C-21D).

These results are congruous with the results of gene expression and show that the level of pathogenesis of HCVmut7 is comparable to control non-infected cells, in accordance with its low level of viral replication.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An attenuated Hepatitis C Virus (HCV) variant, comprising: a plurality of synonymous mutations in the coding regions of the HCV genome, wherein said synonymous mutations are designed to disrupt RNA secondary structures and regulatory sequences critical for the viral life cycle; wherein the synonymous mutations preserve the amino acid sequence of the viral proteins; and wherein the attenuated HCV variant exhibits reduced viral fitness compared to the wild-type HCV.

2. The attenuated HCV variant of claim 1, wherein said reduced viral fitness comprises lower RNA levels, reduced infection percentage, smaller viral spread in liver cells or a combination thereof.

3. The attenuated HCV variant of claim 1, wherein the attenuated HCV variant demonstrates genomic stability over time without significant reversion to the wild-type sequence.

4. The attenuated HCV variant of claim 1, wherein the synonymous mutations are introduced in regions with high selection for strong RNA folding.

5. The attenuated HCV variant of claim 1, wherein the synonymous mutations result in a change in local folding energy (LFE) by at least 15%.

6. The attenuated HCV variant of claim 1, wherein the synonymous mutations are introduced in regions with underrepresented sequences.

7. The attenuated HCV variant of claim 1, wherein the synonymous mutations are introduced in the NS5 gene.

8. The attenuated HCV variant of claim 1, wherein said NS5 gene before said synonymous mutations comprises SEQ ID NO: 22, and wherein said synonymous mutations within SEQ ID NO: 22 comprise substitutions at at least the group of codons consisting of:
a. 1, 43, 106, 127, 169, 190, 211, 232, 266, 286, 327, 368, 388, 430, 468, 489, 510, 531, 552, 573, 594, 615, 657, 678, 762, 783, 804, 825, 846, 909, 951, and 993;
b. 9, 16, 23, 30, 37, 44, 51, 58, 65, 79, 86, 100, 107, 114, 121, 135, 142, 149, 156, 163, 170, 198, 205, 212, 233, 243, 250, 253, 260, 267, 280, 300, 307, 314, 321, 328, 341, 348, 362, 383, 389, 396, 410, 417, 424, 431, 438, 448, 455, 469, 476, 483, 490, 497, 504, 532, 539, 546, 560, 567, 574, 581, 602, 609, 623, 630, 637, 644, 651, 665, 672, 693, 707, 721, 728, 749, 763, 770, 777, 784, 791, 798, 805, 812, 819, 826, 833, 840, 847, 854, 875, 882, 889, 896, 903, 910, 917, 931, 945, 952, 966, 973, 987, and 994;
c. 132, 133, 138, 139, 140, 141, 145, 146, 203, 204, 206, 208, 209, 213, 214, 215, 216, 217, 486, 488, 490, 491, 493, 494, 496, 497, 498, and 500;
d. 105, 106, 107, 109, 110, 112, 113, 114, 115, 116, 117, 132, 133, 138, 139, 140, 141, 145, 146, 203, 204, 206, 208, 209, 213, 214, 215, 216, 217, 333, 334, 335, 337, 338, 339, 340, 341, 342, 343, 345, 346, 347, 349, 366, 368, 371, 372, 373, 376, 377, 378, 379, 380, 486, 488, 490, 491, 493, 494, 496, 497, 498, 500, 620, 621, 625, 627, 628, 629, 631, 632, 633, 723, 727, 728, 729, 730, 733, 734, 737, 751, 753, 757, 762, 764, 766, 836, 837, 838, 839, 840, 842, 845, 846, 848, 850, 981, 983, 984, 985, 987, 989, 990, 992, and 993;
e. 105, 106, 107, 109, 110, 113, 114, 115, 116, 117, 120, 132, 133, 139, 140, 141, 143, 145, 146, 203, 204, 206, 209, 212, 213, 214, 215, 216, 217, 333, 334, 335, 337, 338, 339, 340, 341, 342, 343, 344, 345, 346, 349, 366, 368, 369, 370, 371, 372, 376, 378, 379, 380, 486, 489, 491, 492, 493, 496, 497, 498, 499, 500, 621, 625, 627, 628, 629, 630, 631, 632, 633, 725, 728, 729, 730, 731, 733, 735, 737, 751, 756, 760, 762, 763, 764, 837, 838, 839, 840, 842, 844, 845, 846, 847, 850, 983, 984, 986, 987, 989, 990, 992, 993, and 995;
f. 105, 106, 107, 109, 110, 113, 114, 115, 116, 117, 120, 132, 138, 139, 141, 143, 144, 145, 146, 203, 206, 209, 210, 211, 213, 214, 215, 216, 217, 333, 334, 335, 336, 337, 339, 340, 341, 342, 343, 344, 346, 347, 349, 368, 369, 371, 373, 374, 376, 377, 378, 379, 380, 486, 487, 492, 493, 495, 496, 497, 498, 499, 500, 621, 625, 627, 628, 629, 630, 631, 632, 633, 723, 725, 726, 728, 729, 730, 734, 737, 750, 751, 753, 760, 762, 763, 837, 838, 840, 841, 842, 843, 844, 845, 846, 850, 982, 984, 985, 986, 987, 989, 990, 992, and 995; or
g. 12, 14, 21, 25, 46, 59, 60, 91, 95, 109, 126, 163, 224, 225, 241, 242, 282, 293, 303, 319, 320, 343, 347, 349, 350, 380, 400, 407, 415, 440, 457, 460, 461, 464, 467, 469, 478, 479, 480, 507, 508, 530, 559, 560, 608, 617, 642, 655, 663, 671, 686, 725, 736, 737, 741, 777, 784, 793, 798, 820, 824, 834, 856, 870, 885, 890, 898, 902, 903, 911, 965, 975, 986, 993, and 1053.

9. The attenuated HCV variant of claim 8, wherein said synonymous mutations comprise at least one group of mutations within SEQ ID NO: 22 selected from:
a. C3T, C144G, C345G, C408T, G534A, T595C, G597T, A663C, C726G, G831C, C894T, C1026A, T1149A, T1209G, C1338T, G1458A, A1521C, C1584T, C1647G, G1710T, A1791G, T1857G, C1920T, T2055C, C2118T, A2370T, T2445A, G2517C, C2578A, G2580A, C2652A, T2856C, G2997A, and A3159T;
b. T36C, C57T, T79C, C102T, G123A, C147T, C168G, C189T, C210A, G258C, C279T, C324G, G348T, G369A, A390C, A432G, T451A, C452G, C474T, A495G, G516A, T537G, C624T, G645C, A666C, C729T, C759T, C780T, T792C, G813A, C834T, C876G, A939G, G960T, A984C, T1008C, C1029G, A1068T, C1089T, C1131T, T1194C, G1212A, T1233C, G1278T, A1299G, G1320A, T1341G, T1365A, C1395T, C1419T, T1459C, G1461T, C1482T, C1503T, A1524T, T1545C, A1564C, G1566C, C1650T, T1669C, G1671T, A1690C, A1692G, C1734T, G1764C, A1794C, C1818G, T1881C, C1900A, T1944C, C1968T, T1989A, C2010T, G2034A, C2079T, G2100A, G2163C, A2203T, G2204C, G2247A, T2268C, C2331T, A2373G, G2397A, C2424G, T2448A, A2469G, C2499T, T2518C, G2520T, A2541G, A2562G, T2583C, T2610G, C2634A, A2655C, C2682T, A2751G, C2772T, A2791C, T2814C, C2838T, A2859T, C2886G, C2931G, C2976T, C3000T, C3051T, T3078C, T3135C, and C3162T;
c. A396G, C397T, G399A, G414T, A417G, T420C, T423A, T435G, T438G, A609C, A612C, G618A, C624T, C627A, T639G, G642A, G645A, C646A, C649A, C651G, G1458A, A1464C, C1470T, T1473G, T1479C, C1482T, G1488C, T1489C, A1494C, and T1500C;
d. C315T, C318T, G321A, C327G, C330T, A334C, G339A, G342C, C345T, G348A, G351A, A396G, C397T, G399A, G414T, A417G, T420C, T423A, T435G, T438G, A609C, A612C, G618A, C624T, C627A, T639G, G642A, G645A, C646A, C649A, C651G, C999T, C1002T, T1005C, T1011C, T1014C, T1017C, C1018T, C1020G, C1023G, C1026T, C1029A, G1035A, C1038A, G1041A, T1047A, A1098G, C1102T, C1104G, G1113T, C1116T, C1119T, T1128C, C1131T, G1134A, C1137G, C1140G, G1458A, A1464C, C1470T, T1473G, T1479C, C1482T, G1488C, T1489C, A1494C, T1500C, G1860A, A1863G, C1875T, T1881G, C1884T, T1887C, C1891T, C1893G, C1896T, C1899T, T2169C, C2181T, A2184G, A2187G, G2190T, C2199T, C2202T, T2211G, A2253G, T2259C, C2271T, T2286C, A2292G, C2296T, G2508C, T2511C, G2514C, G2517C, T2518C, G2526A, C2533A, C2535A, C2536A, C2538G, C2544T, G2550C, A2943G, A2949G, G2952C, C2955T, C2961T, A2967G, T2970C, C2976T, and T2979C;
e. C315T, C318T, G321A, C327G, C330T, G339A, G342T, C345T, G348A, G351A, G360A, A396G, C397T, A417G, T420C, T423G, A429G, T435G, T438G, A609C, A612C, G618C, C627A, G636A, T639G, G642T, G645C, G648T, C649A, C651G, C999A, C1002T, T1005G, T1011C, T1014C, T1017C, C1018T, C1020G, C1023G, C1026T, C1029A, G1032A, G1035A, C1038G, T1047A, A1098G, C1104G, G1107A, A1110G, G1113C, C1116T, T1128C, G1134A, C1137G, C1140T, G1458A, C1467T, T1473C, T1474C, T1479C, G1488C, T1489C, A1494T, C1497T, T1500C, A1863G, C1875T, T1881A, C1884T, T1887A, C1890T, C1893G, C1896A, C1899G, A2173C, A2175G, A2184T, A2187G, G2190C, C2193T, C2199T, A2203T, G2204C, C2205A, T2211G, A2253G, T2268C, A2280T, T2286C, G2289A, A2292G, T2511C, G2514C, G2517C, T2518C, G2526T, C2532A, C2533A, C2535A, C2536A, C2538A, A2539C, A2541G, G2550C, A2949G, G2952T, T2958A, C2961T, C2965A, T2970C, C2976T, T2979C, and G2985A;
f. C315T, C318T, G321A, C327G, C330T, G339A, G342C, C345A, G348A, G351A, G360A, A396C, G414T, A417G, T423G, A429G, A432G, T435A, T438G, A609C, G618A, C627T, G630A, G633A, T639G, G642A, G645T, G648T, C649A, C651G, C999T, C1002T, T1005G, T1008G, T1011G, T1017C, C1018T, C1020G, C1023A, C1026T, C1029A, G1032A, C1038G, G1041A, T1047C, C1102T, C1104G, G1107A, G1113C, C1119T, G1122A, T1128C, C1131T, G1134A, C1137A, C1140A, G1458A, T1459C, T1474C, T1479C, G1485T, G1488C, T1489C, G1491A, A1494T, C1497T, T1500C, A1863G, C1875T, T1881A, C1884T, T1887C, C1890T, C1891T, C1893G, C1896A, C1899T, T2169C, A2173C, A2175G, T2178C, A2184T, A2187G, G2190C, C2202T, T2211G, G2250A, A2253G, T2259C, A2280T, T2286C, G2289A, T2511C, G2514C, T2518C, C2523T, G2526T, C2527A, G2529A, C2532A, C2533A, C2535A, C2536A, C2538A, G2550C, G2946C, G2952T, C2955T, T2958C, C2961T, A2967G, T2970C, C2976T, and G2985A; and
g. T36C, C42G, T63C, T75C, G138C, C177T, C180G, T273C, G285A, C327G, G378T, C489G, A672C, T675C, C723G, C726A, A846G, A879C, C909T, G957C, G960A, C1029A, G1041A, T1047C, C1050A, C1140A, G1200C, C1221A, T1245C, G1320A, T1369A, C1370G, C1380T, T1383C, G1392C, C1401G, A1407T, T1434C, C1437A, T1440C, A1521C, A1524C, T1590C, A1677C, C1680A, T1824C, G1851A, T1926C, T1965C, T1989A, T2011C, G2013C, T2058C, A2173C, A2175G, G2208A, T2211G, T2223C, C2331G, T2352C, G2379A, C2394T, C2460A, T2472C, A2502C, T2568C, T2610A, A2655C, C2670T, T2694C, A2706G, C2709T, T2733C, G2895C, G2925C, T2958C, T2979C, and A3159C.

10. The attenuated HCV variant of claim 8, comprising any one of SEQ ID NO: 24-30.

11. The attenuated HCV variant of claim 10, wherein a NS5 gene of said HCV variant comprises any one of SEQ ID NO: 24-30.

12. The attenuated HCV variant of claim 8, wherein said NS5 gene
a. comprises fewer than 298 synonymous codon substitutions;
b. does not comprise codon substitutions at all of the following codons in SEQ ID NO: 22: 10, 13, 18, 19, 35, 36, 42, 51, 57, 59, 60, 65, 67, 74, 80, 82, 89, 90, 94, 96, 100, 105, 106, 109, 113, 118, 119, 120, 136, 142, 148, 150, 154, 156, 170, 172, 175, 179, 185, 187, 192, 193, 194, 195, 201, 206, 208, 211, 212, 221, 223, 227, 228, 232, 233, 240, 243, 251, 257, 258, 259, 262, 266, 269, 271, 273, 278, 280, 291, 293, 297, 310, 315, 316, 317, 318, 330, 342, 352, 374, 408, 409, 415, 417, 418, 420, 421, 422, 424, 427, 441, 463, 465, 466, 470, 473, 474, 484, 486, 489, 494, 501, 504, 517, 521, 527, 535, 536, 538, 541, 549, 552, 553, 555, 557, 568, 569, 570, 571, 572, 573, 574, 580, 581, 584, 585, 587, 588, 591, 593, 595, 598, 603, 604, 609, 611, 617, 623, 624, 626, 628, 630, 635, 636, 637, 640, 643, 651, 659, 660, 661, 666, 677, 687, 689, 690, 691, 694, 696, 698, 699, 702, 706, 707, 709, 715, 722, 724, 726, 727, 729, 731, 734, 740, 745, 746, 748, 754, 757, 758, 761, 765, 768, 774, 776, 785, 788, 789, 792, 797, 798, 803, 805, 806, 807, 808, 810, 811, 812, 814, 815, 816, 822, 825, 827, 829, 830, 831, 833, 834, 836, 838, 841, 848, 849, 851, 853, 861, 862, 865, 868, 869, 870, 873, 876, 877, 879, 894, 895, 896, 905, 915, 918, 919, 922, 924, 925, 928, 933, 937, 939, 941, 943, 944, 945, 948, 949, 950, 951, 955, 956, 958, 959, 962, 963, 964, 967, 969, 970, 973, 975, 979, 981, 982, 987, 988, 991, 992, 995, 996, 998, 999, 1002, 1012, 1014, 1017, 1020, 1022, 1023, 1024, 1025, 1028, 1029, 1031, 1033, 1035, 1036, 1041, 1042, 1043, 1044, 1045, 1048, 1050, 1051, 1054, 1055, and 1057;
c. does not comprise SEQ ID NO: 31; or
d. a combination thereof.

13. The attenuated HCV variant of claim 1, being a mutant of a natural isolate, being a synthetic virus, comprising a nucleotide acid selected from: single strand RNA (ssRNA), (dsRNA) double strand RNA, single strand DNA (ssDNA) and double strand DNA (dsDNA) or both; optionally wherein the said nucleotide acid is ssRNA.

14. A vaccine composition comprising the attenuated HCV variant of claim 1 and a pharmaceutically acceptable carrier, excipient or adjuvant.

15. A method for eliciting a protective immune response against HCV in a subject comprising administering to said subject a prophylactically effective dose of the vaccine of claim 14, thereby eliciting a protective immune response in the subject; optionally wherein said eliciting a protective immune response is vaccinating.

16. A method for designing an attenuated Hepatitis C Virus (HCV) variant based on mRNA folding, comprising:
a. obtaining a multiple sequence alignment (MSA) of HCV strains;
b. using a computational algorithm to identify regions within the HCV genome with significant selection for strong or weak RNA folding;
c. introducing a plurality of synonymous mutations in the identified regions to alter the local folding energy (LFE) by at least 15%, thereby disrupting the RNA secondary structures critical for the viral life cycle, while preserving the amino acid sequence of the viral proteins;
d. generating the attenuated HCV variant with the introduced synonymous mutations;
e. evaluating the viral fitness of the attenuated HCV variant by measuring RNA levels, infection percentage, and/or viral spread in liver cells;
f. confirming the genomic stability of the attenuated HCV variant over time without significant reversion to the wild-type sequence;
thereby designing an attenuated HCV variant.

17. A method for designing an attenuated Hepatitis C Virus (HCV) variant based on underrepresented sequences, comprising:
a. obtaining a multiple sequence alignment (MSA) of HCV strains;
b. using a computational algorithm to identify underrepresented (UR) sequences within the HCV genome;
c. introducing a plurality of synonymous mutations to insert the identified UR sequences into their corresponding regions, thereby disrupting the RNA regulatory elements critical for the viral life cycle, while preserving the amino acid sequence of the viral proteins;
d. generating the attenuated HCV variant with the introduced synonymous mutations;
e. evaluating the viral fitness of the attenuated HCV variant by measuring RNA levels, infection percentage, and/or viral spread in liver cells;
f. confirming the genomic stability of the attenuated HCV variant over time without significant reversion to the wild-type sequence;
thereby designing an attenuated HCV variant.

18. The method of claim 16, wherein at least one of:
a. the multiple sequence alignment (MSA) is obtained from a database comprising at least 100 complete HCV strains;
b. the synonymous mutations are introduced only in codons whose frequency in the MSA column is at least 10%;
c. the evaluation of viral fitness includes measuring the size of viral foci in liver cells;
d. the evaluation of viral fitness includes measuring the percentage of infected liver cells over a period of 4 weeks;
e. the confirmation of genomic stability includes deep sequencing of the NS5A and NS5B genes of the attenuated HCV variant; and
f. the synonymous mutations are introduced in the NS5 gene.

19. The method of claim 16, wherein at least one of:
a. the computational algorithm used to identify regions with significant selection for strong or weak RNA folding includes a sliding window approach with a window length of 39 nucleotides;
b. the synonymous mutations are designed to change the local folding energy (LFE) by at least 15% in regions with significant selection for strong RNA folding; and
c. the synonymous mutations are designed to change the local folding energy (LFE) by at least 15% in regions with significant selection for weak RNA folding.

20. The method of claim 17, wherein at least one of:
a. the computational algorithm used to identify underrepresented (UR) sequences includes synonymous codon permutations and synonymous dinucleotide permutations; and
b. the synonymous mutations are designed to insert underrepresented sequences in both frame 1 and frame 2 of the HCV genome.
